(19) United States
(12) Patent Application Publication
(10) Pub. No.: US 2009/0123501 A1
(43) Pub. Date: May 14, 2009

(54) INHIBITION OF THE SH2-DOMAIN CONTAINING PROTEIN TYR-PHOSPHATASE, SHP-1, TO ENHANCE VACCINES

(75) Inventors: Jonathan Levitt, Houston, TX (US); Indu R. Ramachandran, Houston, TX (US); Kevin M. Slawin, Houston, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine, Houston, TX (US)

(21) Appl. No.: 12/122,146

(22) Filed: May 16, 2008

Related U.S. Application Data
(60) Provisional application No. 60/938,545, filed on May 17, 2007.

(51) Int. Cl. A61K 39/00; A61K 35/12; A61K 31/7088

(52) U.S. Cl. 424/277.1; 424/93.7; 514/44; 424/184.1

(57) ABSTRACT

The invention describes the use of dendritic cell vaccines, wherein SHP-1 expression or activity is modulated in the dendritic cell. In particular, the invention provides dendritic cells (DC) transduced with an SHP1-shRNA adenovirus, or dominant negative (dn-SHP-1) or constitutively active (ca-SHP-1), and pulsed with an antigen. The methods and compositions of the invention are used for the prevention and/or treatment of cancers, other cell proliferation diseases and conditions, diseases caused by a pathogen, or autoimmune disorders.

### CROSS-REFERENCE TO RELATED APPLICATIONS

This application claims priority to U.S. Provisional (35 USC § 119(e)) Application Ser. No. 60/938,545, filed on May 17, 2007, which is incorporated by reference herein in its entirety.

### STATEMENT REGARDING FEDERALLY SPONSORED RESEARCH OR DEVELOPMENT

The present invention was developed using federal funds from the Department of Defense New Investigator Award Grant No. PC061027 W81XWH-07-1-0025. The United States Government has certain rights in the invention.

### TECHNICAL FIELD

The present invention generally relates at least to the fields of cell biology, immunology, molecular biology, and medicine, in some cases cancer. Specifically, the invention concerns methods and/or compositions for enhancing a vaccine, including for the treatment and/or prevention of cancer, in certain cases.

### BACKGROUND OF THE INVENTION

A vaccine is a preparation that is used to improve immunity to a particular disease. The immune system recognizes vaccine components (antigens), mounts a response against those antigens, and can generate immunological memory to facilitate protection on future encounters with the antigen. For example, vaccines have contributed to the eradication of smallpox, one of the most contagious and deadly diseases known to man. Vaccines have been used to treat other diseases such as rubella, polio, measles, mumps, chickenpox, typhoid and in some cases cancer.

In 2005, there were an estimated 230,090 newly diagnosed cases of prostate cancer (PCa) in the United States, accounting for 33% of all cancers affecting men (Macvicar et al., 2005). Organ-confined early-stage prostate cancer is successfully managed with surgical and radiation therapies leading to long term patient survival. Despite the effectiveness of these localized therapies, 5-10% of patients develop metastatic disease within 8 years of radical prostatectomy. Standard treatments of metastatic disease by androgen ablation are successful at suppressing metastasis but ultimately result in the evolution of androgen-independent tumors within 2 years. Currently, FDA approved treatments for disseminated hormone refractory disease are limited to chemotherapies, the best of which, the combination of docetaxel and estramustine, results in a median patient life expectancy of only 18.9 months (Petrylak et al., 2004). The limited scope of treatment options for late-stage prostate cancer and the relatively short term efficacy of the existing treatments, highlights the need for research and innovation aimed at developing more effective therapeutic modalities.

One emerging strategy for treatment of late-stage disease of any type of cancer is adjuvant stimulation of anti-tumor adaptive immune responses using dendritic cells (DC) (Banchereau et al., 2005, Vieweg et al., 2005). The use of DC to process and present antigen, with or without ectopic expression of various cytokines has shown potential as anti-tumor treatment (Chen et al., 2006; Kantoff, 2005). Early preclinical and clinical trials indicate that tumor “vaccines” are both feasible and safe (Small et al., 2000). These trials also demonstrate only limited efficacy in causing tumor regression despite eliciting measurable systemic T cell responses against prostate cancer (Chen et al., 2006; Schuler-Thurner et al. 2002; Su et al., 2005). However, these “first-generation vaccines” have given a solid foundation for the use of immunotherapy's in the treatment of cancer.

### Initiation of Adaptive T Cell Mediated Immunity

Adaptive immune responses require activation of T cells (Janeway, 2001). The differentiation and proliferation of specific T cell subsets is determined by the interaction of naïve T cells with specialized antigen presenting cells, DC. Dendritic cells are unique in their ability to provide antigen specific ligation through the T cell receptor and concomitant stimulation through one or more co-receptors as well as the ability to express a range of inflammatory cytokines (Banchereau and Steinman, 1998). The specific mixture of DC derived signals dictates the type of T cell response generated.

In order to initiate T cell responses, DC must undergo a genetic maturation process. This process is driven by environmental “danger signals” through Toll-like receptors (TLR) by a range of compounds that are typically expressed by microbial pathogens including LPS, and unmethylated CpG DNA (Banchereau et al., 2000). The maturation process comprises the up-regulation of costimulatory molecules at the cell surface (members of the B7 family), an increase in MHC-peptide expression and the production of inflammatory cytokines such as TNFα and IL-12 (Cella et al., 1997; Cella et al. 1999). Mature DC also up-regulate expression of the chemokine receptor CCR7 that enables them to migrate to draining lymph nodes where the T cell activation occurs (Sallusto et al., 1999).

### Dendritic Cells as Cancer Vaccines

Prostate cancer utilizes an array of strategies to evade the immune system, including down regulation of MHC class I expression and induction of DC apoptosis or dysfunction (Bander et al., 1997; Pirtskhalaishvili et al., 2000; Schuler and Steinman, 1997). Since DC are the key initiators of T cell responses it makes them an ideal platform for the development of cancer vaccines (Nestle et al., 2001). Monocytic DC precursors can be purified from peripheral blood and can be differentiated easily into immature DC in vitro by culture with the cytokines GM-CSF and IL-4 (Thurner et al., 1999). DC can also be loaded with specific antigens and matured in vitro by the addition of cytokine cocktails and/or TLR ligands (Napolitani et al., 2005). Recent clinical trials using DC vaccines in the treatment of late-stage prostate cancer, however, have shown only limited success, suggesting there is a need to further improve DC as an antigen delivery platform (Ridgway, 2003).

In nature, adaptive immune responses are tempered by a number of inhibitory pathways that maintain a fine balance within the body between appropriate immunity and the generation of autoimmune responses (Long, 1999). Several of these dampening mechanisms are mediated through DC. DC have a short lifespan and a transient activation state within lymphoid tissues (Hou and Van Parijs, 2004). Less than 24 hours following exposure to lipopolysaccharide (LPS), DC terminate synthesis of the Th1-polarizing cytokine, IL-12, and become refractory to further stimuli (Langenkamp et al., 2000), limiting their ability to activate cytotoxic T lymphocytes (CTLs). Other studies indicate that the survival of antigen-pulsed DC within the draining lymph node (LN) is limited to only 48 hours following their delivery (Hermans et al., 2000). These findings underscore the need for improving the function of DC for use as vaccines by enhancing and/or prolonging their activation state and by increasing their functional life span.

### Role of SHP-1 in Dampening DC Function

The protein tyrosine phosphatase Src homology region 2 domain-containing phosphatase-1 (SHP-1) is a cytosolic protein tyrosine phosphatase expressed primarily in haemopoietic cells (Matthews et al., 1992). SHP-1 is recruited to the cell membrane by phospho-immunoreceptor tyrosine-based inhibitory motifs (ITIM) present in the cytoplasmic tails of a number of inhibitory receptors including the immunoglobulin-like transcript family (ILT), inhibitory Fcγ family, the leukocyte immunoglobulin-like receptor family (LIR), and the signaling lectin family (SigLec) (Allan et al., 2000; Lock et al., 2004; Ravetch, 1997; Yokoyama, 1998). Upon ligation of their specific ligands, inhibitory receptors phosphorylate their ITIM domains and initiate SHP-1 recruitment (Zhang et al., 2000). Once recruited to the membrane, SHP-1 can interact with and dephosphorylate a wide range of signaling molecules including members of the Src-family of protein tyrosine kinases (PTK), downstream members of the IL-1R/Toll-like receptors (TLR) signaling pathway, JAK/STAT family members, G protein coupled factor Vav and PI3K (FIG. 1) (Cuevas et al., 1999; Cambier, 1997; Stebbins et al., 2003; Thomas, 1995; Yeung et al., 1998).

Dendritic cell activation and maturation rely on signaling through NFκB and MAPK pathways mediated predominantly by TLR and CD40 ligation. These activating signals lead to inhibition of apoptosis and DC survival, as well as upregulation of Th1 cytokine production (IL-12, IFNγ) and surface expression of MHC class II molecules, and T cell co-stimulatory ligands (Banchereau et al., 1998). SHP-1 is known to dampen TLR mediated signals in macrophages and B cells and potentially plays this function in DC (Zhang et al., 2000). Also, in normal immune responses, T cells activated by DC secrete stimulatory cytokines (IFNγ) that have paracrine positive feedback effects on DC leading to the propagation of immune responses. However, under normal circumstances T cells also secrete cytokines like IL-10 and IL-21 that dampen the immune response by acting on DC. IL-10 and IL-21 have both been shown to mediate their inhibition of TLR/LPS and IFNγ signals respectively through members of suppressor of cytokine signaling (SOCS) family members and some SOCS have been shown to mediate their function through SHP-1 (Minoo et al., 2004; Qasimi et al., 2006; Strengell et al., 2006; Tsui et al., 1993). Knocking down SOCS in DC leads to potent anti-tumor responses in mouse models.

The importance of SHP-1 inhibitory signals in the immune system are seen in “motheaten” mice (C57BL/6J-Ptpn6me-v/J) which have a loss-of-function mutation in SHP-1. These mice have a profound immunological dysfunction exemplified by an accumulation of myeloid/monocytic cells (macrophages and DC) and severe lethal autoimmunity by 3-9 weeks of age (Tsui et al., 1993). This phenotype indicates that SHP-1 modulates the initiation of adaptive immune responses and indicates that it is a useful target for enhancing the function of DC based vaccines.

### SUMMARY OF THE INVENTION

The present invention is directed to systems, compositions and methods that are utilized for vaccines, including for enhancing vaccines. In some cases, the invention concerns enhancing dendritic cell-based vaccines, including dendritic cell-based vaccines that comprise an antigen, in at least some cases. In particular embodiments the invention is for cancer therapy and/or prevention for an individual, although in other cases the invention is for therapy and/or prevention of disease caused by pathogen or an autoimmune disease. In particular cases, the invention concerns dendritic cell vaccines that provide cancer therapy and/or prevention to an individual with any type of cancer. In specific embodiments, the invention is useful for prostate, pancreatic, lung, brain, breast, liver, colon, uterine, cervical, testicular, skin, bone, spleen, thyroid, stomach, anal, gall bladder, or esophageal cancer, for example. In specific embodiments, the individual is a mammal, such as a human, dog, cat, horse, pig, sheep, mouse, or goat, for example.

In certain embodiments the invention concerns compositions and methods for an individual that has cancer, has metastatic cancer, is suspected of having cancer, or is at high risk for developing cancer. The therapy of the invention may be delivered to the individual at any point of having cancer, and in specific embodiments the individual is also given an additional therapy for cancer, including a cancer therapeutic and/or another vaccine. In particular cases, the additional therapy is delivered to the individual before the therapy/prevention composition/methods of the invention, after the therapy/prevention composition/methods of the invention, and/or during the therapy/prevention composition/methods of the invention. In certain cases the cancer is resistant to one or more therapies, including acquired resistance or de novo resistance.

In particular embodiments of the invention, the dendritic cell vaccines are administered to an individual to prevent and/or treat a disease caused by a pathogen, including a bacteria, virus, protozoa, parasite, or yeast, for example. In specific embodiments the invention is useful for treating bacterial or viral pneumonia, influenza, dysentery, typhoid fever, diphtheria, syphilis, tuberculosis, Herpes simplex, malaria, hepatitis, polio, cholera, rotavirus, black plague, SARS, rabies, mumps, smallpox, encephalitis, chickenpox, Ebola, hand, foot, mouth disease, mad cow disease, whooping cough, yellow fever, lyme disease, botulism, septicemia, or HIV, for example.

In certain alternate embodiments of the present invention, the dendritic cell vaccines may be administered to a subject to prevent and/or treat an immune disorder. Such disorders may include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, psoriasis, rheumatoid arthritis, scleroderma, Sjogren's syndrome, and autoimmune thyroiditis; complications of cancer, hemodialysis, and extracorporeal circulation; viral, bacterial, fungal, parasitic, protozoal, and helminthic infections; and trauma.

In particular embodiments of the invention, there are two different methods of SHP-1 modulation: a) knocking down the amount of endogenous SHP-1 protein expressed in a dendritic cell (for example, by shRNA); or b) inhibiting or augmenting endogenous SHP-1 function using exemplary dominant negative (dn)-SHP-1 or constitutively active (ca)-SHP-1 mutants, respectively. The dn and ca mutants do not change the level of endogenous SHP-1 protein in the cell, in particular aspects of the invention, but overcome its normal function by competing for substrate without performing its catalytic activity (as for dn-) or by providing continuous SHP-1 catalytic activity in the absence of the normal regulatory mechanisms (as for ca-), for example.

In certain embodiments of the present invention, there is a method of enhancing a dendritic cell based vaccine, comprising administering to an individual a SHP-1 modulatory agent or a dendritic cell comprising a SHP-1 modulatory agent and, in some cases an antigen is also administered in a dendritic cell or outside a dendritic cell. In specific embodiments, an antigen and a vector carrying a shRNA or SHP-1 mutant construct sequence that modulates SHP-1 function is administered. In certain embodiments, the antigen is a tumor antigen. In specific cases, the shRNA or SHP-1 mutant construct inhibits SHP-1 function. In certain embodiments, the exemplary shRNA is selected from the group consisting of: [Ad5-shRNA#1149 (SEQ ID NO:11)] and [Ad5-shRNA#272 (SEQ ID NO:12)]. In other embodiments, the exemplary SHP-1 mutant construct is selected from the group consisting of [dn-SHP-1 (SEQ ID NO:8)] and [ca-SHP-1 (SEQ ID NO:9)]. In particular embodiments, SEQ ID NO:7, SEQ ID NO:8, and SEQ ID NO:9 are SHP-1 specific mRNA coding sequences that are preceded by influenza virus hemagglutinin 30-bp sequence, for example, used as an epitope tag for distinguishing endogenous SHP-1 and transfected SHP-1 in cells. In specific embodiments, the shRNA comprises or consists of sequence [Ad5-shRNA#1149 (SEQ ID NO:11)]. In other embodiments, the shRNA comprises or consists of sequence [Ad5-shRNA#272 (SEQ ID NO:12)]. In specific cases, the SHP-1 mutant construct comprises or consists of sequence [dn-SHP-1 (SEQ ID NO:8)]. In other embodiments, the SHP-1 mutant construct comprises or consists of sequence [ca-SHP-1 (SEQ ID NO:9)].

In specific embodiments, sequences employed in the invention or targeted by the invention are human sequences. For example, the shRNA sequence may correspond to a human SHP-1, the tumor antigen may be obtained from a human sequence, and so forth.

Although in some embodiments a dendritic cell is manipulated ex vivo to encompass a SHP-1 modulatory agent, in some embodiments the SHP-1 modulatory agent is taken up by a dendritic cell in vivo, following delivery of the SHP-1 modulatory agent (for example, an RNA or DNA) itself to an individual. In certain aspects, SHP-1 is modulated in an individual's dendritic cells by injecting a DNA or RNA that expresses the SHP-1 modulating agent and, optionally, an antigenic sequence, which may or may not be on the same nucleic acid molecule. In specific embodiments, the injected sequences are expressed under the control of a dendritic cell-specific promoter (CD11c for example). In this manner, one bypasses the need to purify an individual's dendritic cells, which may be considered to be a time consuming and expensive process, and modify the individual's dendritic cells in vivo. For this exemplary embodiment, a single “vaccine formulation” could be mass produced and used for all individuals.

In some embodiments of the invention, a nucleic acid sequence that is or encodes a SHP-1 modulatory agent is delivered to a cell. In other embodiments of the invention, a nucleic acid sequence that encodes an antigen, such as a tumor antigen, is delivered to a cell.

The particular shRNA or SHP-1 mutant construct of the invention may consist of particular sequences or, in other aspects of the invention, there may be additional sequences in the nucleic acids. In particular embodiments, the shRNA is isolated and cloned into a vector. Though the vector may be of any suitable kind, in specific embodiments, the vector is an adenoviral vector, for example, the adenoviral pAd-BLOCK-iT-DEST or pAdTrack-CMV vector. In specific aspects, the SHP-1 modulatory agent is a shRNA that is directed against SHP-1 function. In alternative embodiments, the SHP-1 modulatory agent is a SHP-1 stimulator that comprises a constitutively active SHP-1 gene product. In further embodiments, an anti-cancer agent(s) comprises one or more of the nucleic acids of the invention. In specific aspects of the invention, biological functional equivalents to the shRNA may comprise a oligonucleotide that has been engineered to contain distinct sequences while at the same time retaining the capacity to encode a particular peptide. In some embodiments, the enhanced dendritic cell-based vaccine of the invention comprises an antigen. Although the antigen may be of any suitable kind, in specific embodiments the antigen is a tumor antigen. However, in particular cases the antigen is an antigen for an autoimmune disorder or a disease caused by a pathogen.

In some embodiments of the invention, the exemplary antigen is further defined as comprising a sequence selected from the following group consisting of STEAP186-192 (SEQ ID NO: 2), STEAP84-91 (SEQ ID NO: 3), STEAP327-335 (SEQ ID NO: 4), STEAP262-270 (SEQ ID NO: 5), PSCA29-37 (SEQ ID NO: 6), and a combination thereof. An experimental control antigen from chicken ovalbumin is provided in OVA258-265 (SEQ ID NO: 1). In a specific embodiment, the amino acid sequence of the antigen utilizes human-specific sequences and the actual antigen used is dependent on the specific pathogen, autoimmune disorder, or type of cancer treated, for example.

In certain embodiments of the present invention, there is a method for modulating an immune response in an individual using dendritic cell based vaccines comprising administering a SHP-1 modulatory agent or a dendritic cell comprising a SHP-1 modulatory agent. In specific embodiments, the SHP-1 modulatory agent is a SHP-1 inhibitory agent, such as a shRNA that is cloned in a vector; the vector is transduced into dendritic cells to an individual. In specific embodiments, the vector is an adenoviral vector. In certain cases, the dendritic cell vaccines are loaded with an antigen. In certain embodiments the antigen is further defined as a tumor antigen. In additional embodiments, the SHP-1 modulatory agent inhibits SHP-1 function. In specific cases, the individual has an autoimmune disease. In other cases, the individual has cancer.

Another embodiment of the present invention includes a composition comprising shRNA-loaded dendritic cell-based vaccine or SHP-1 mutant-loaded dendritic cell-based vaccine, wherein upon administration of the composition induces a pro-inflammatory immune response that inhibits hyperproliferative cell growth. In specific embodiments, the hyperproliferative cell is a cancer cell, such as a tumor cell. For example, the cancer cell is a melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell. In specific embodiments the antigen-presenting cells are dendritic cells.

In certain embodiments of the invention the individual is delivered an additional cancer therapy such as one that comprises chemotherapy, immunotherapy, radiation, surgery, or a combination thereof. In specific embodiments, the cancer is prostate cancer. In other embodiments, the cancer is prostate cancer.

In additional embodiments of the present invention, the individual has an autoimmune disease. In other embodiments of the present invention, the individual has cancer. In specific cases, the dendritic cell based vaccine is administered to the individual simultaneously or subsequently to the administration of a cancer therapy that is not the vaccine of the present invention.

In certain embodiments of the present invention, there is a method of producing a dendritic cell-based vaccine, comprising transducing a dendritic cell with a SHP-1 modulatory agent, including a vector carrying a shRNA sequence that modulates SHP-1 function, for example, and loading the dendritic cell with an antigen, although in some cases the dendritic cell already comprises the antigen. In certain embodiments, the shRNA inhibits SHP-1 function. In other embodiments, the SHP-1 modulatory agent stimulates SHP-1 function.

Certain embodiments of the present invention include a composition comprising a SHP-1 modulatory agent. In specific embodiments, there is dendritic cell based vaccine comprising a vector containing a SHP-1 modulatory agent, such as a shRNA sequence that modulates SHP-1 function, and, in some aspects, also comprises an antigen. In certain cases, upon administration of the composition to an individual, an immune response of the individual is modulated.

In certain embodiments of the present invention, there is a method of modulating an immune response in an individual comprising employing a dendritic cell, wherein the dendritic cell is transduced with a SHP-1 modulatory agent, such as an adenoviral vector comprising a shRNA that modulates SHP-1 function. In certain embodiments the dendritic cell is loaded with an antigen, wherein the antigen is further defined as a tumor antigen. In certain cases, the SHP-1 modulatory agent stimulates SHP-1 function. In certain cases, the shRNA inhibits SHP-1 function.

In certain embodiments of the present invention, there is a method of enhancing a dendritic cell-based vaccine, comprising administering to an individual a dendritic cell comprising a SHP-1 modulatory agent (for example, a vector carrying a shRNA sequence that modulates SHP-1 function), optionally an antigen, and optionally an additional immune stimulating agent. In certain embodiments, that antigen is a autoimmune antigen. In additional embodiments, the additional immune stimulating agent is comprised of an engineered recombinant receptor comprised of the cytoplasmic domain of CD40 fused to the ligand binding domains of an FK506 derived protein mutant that can bind a dimerizing agent and/or a membrane-targeting sequence (iCD40), or a constitutively active chimeric variant of the signaling molecule Akt (myr_{F}-ΔAkt).

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows the SHP-1 inhibitory pathways in DC.

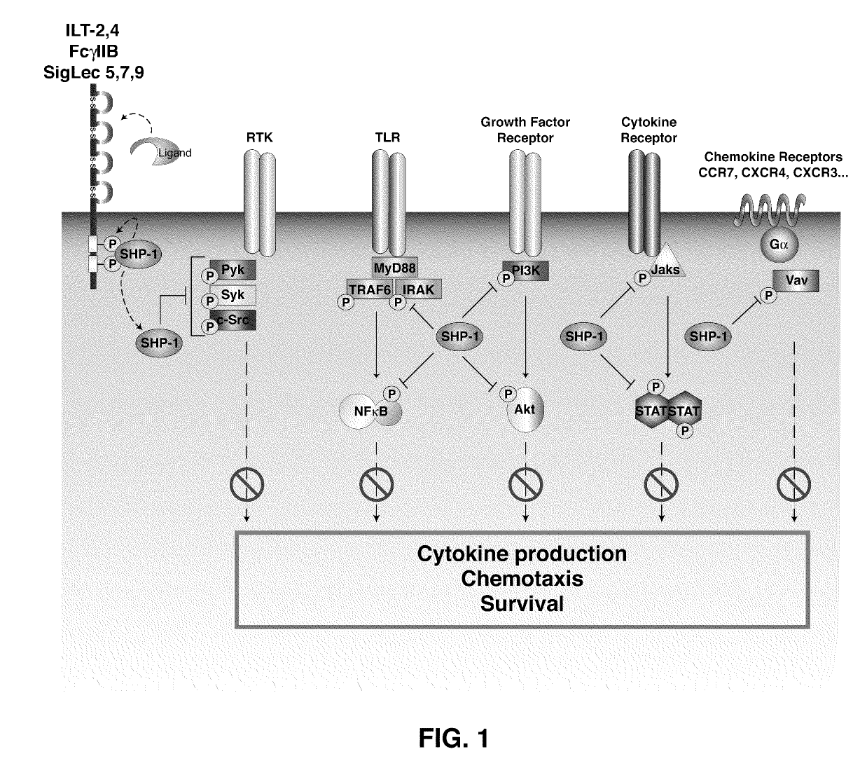

FIGS. 2A-2D show the SHP-1 knock-down by shRNA.

FIG. 3 shows the knock-down of endogenous SHP-1 using high titer Ad5-SHP-1 shRNA.

FIG. 4 shows the comparison of SHP-1 mutant and wild type constructs.

FIGS. 5A-5B show that SHP-1 function modulates NFκB and AP-1 signaling.

FIGS. 6A-6B show that SHP-1 inhibits CCR7-dependent migration in vitro.

FIG. 7 shows that knock down of SHP-1 enhances the migration of DCs out of the footpad.

FIG. 8 shows that SHP-1 knock down enhances DC survival.

FIG. 9 shows that SHP-1 signaling inhibits Akt phosphorylation.

FIG. 10 shows that DC vaccination enhances Th1 skewing of T cells.

FIG. 11 shows that SHP-1 knock down enhances CD8⁺ effectors and CD4⁺ Th1 while inhibiting FOXP3⁺ Treg induction.

FIG. 12 shows luciferase expressing glow tumors.

FIG. 13 shows the relative binding affinities of exemplary, STEAP and PSCA peptides.

FIGS. 14A-141 show that SHP-1 inhibition enhances DC vaccines against TRAMP C2 tumors.

FIGS. 15A-15B show that SHP-1 inhibition enhances DC vaccines against B16 tumors.

FIG. 16 shows the in vivo imaging of subcutaneous B16 tumors in mice. Mice were inoculated s.c. with 10⁵ B16 tumor cells expressing rs-Luc. After 5 days, mice were anaesthetized and injected with 100 ml d-luciferin (15 mg/ml) i.p., 15′ later mice were imaged for 30″ with an IVIS™ Imaging system.

FIG. 17 shows that bone marrow derived DC are matured by LPS. Bone marrow lymphocytes were cultured for 7 days in GM-CSF and IL-4 before further purification on an anti-CD11c column. CD11c⁺ DC were incubated in LPS for 2 days and expression of surface maturation markers was determined by flow cytometry. White curves are cells stained with FITC labeled isotype control, red curves are cells stained either CD40, CD86, or MHC class II specific mAb.

FIG. 18 shows an exemplary preparation of bone marrow derived DC.

FIG. 19 shows an exemplary vaccination protocol for ectopic tumor bearing mice.

### DETAILED DESCRIPTION OF THE INVENTION

### I. Definitions

As used herein, the use of the word “a” or “an” when used in conjunction with the term “comprising” in the sentences and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The term “antigen” as used herein is defined as a molecule that elicits an immune response. This immune response may involve either antibody production, or the activation of specific immunologically-competent cells, or both. An antigen can be derived from organisms, subunits of proteins/antigens, killed or inactivated whole cells or lysates. For example, exemplary organisms include but are not limited to, Helicobacters, Campylobacters, Clostridia, Corynebacterium diphtheriae, Bordetella pertussis, influenza virus, parainfluenza viruses, respiratory syncytial virus, Borrelia burgdorfei, Plasmodium, herpes simplex viruses, human immunodeficiency virus, papillomavirus, Vibrio cholera, E. coli, measles virus, rotavirus, shigella, Salmonella typhi, Neisseria gonorrhea. Therefore, a skilled artisan realizes that any macromolecule, including virtually all proteins or peptides, can serve as antigens. Furthermore, antigens can be derived from recombinant or genomic DNA, in some cases. A skilled artisan realizes that any DNA that contains nucleotide sequences or partial nucleotide sequences of a pathogenic genome or a gene or a fragment of a gene for a peptide that elicits an immune response results in synthesis of an antigen. Furthermore, one skilled in the art realizes that the present invention is not limited to the use of the entire nucleic acid sequence of a gene or genome. It is readily inherent that the present invention includes, but is not limited to, the use of partial nucleic acid sequences of more than one gene or genome and that these nucleic acid sequences are arranged in various combinations to elicit the desired immune response.

The term “antigen-presenting cell” is any of a variety of cells capable of displaying, acquiring, or presenting at least one antigen or antigenic fragment on (or at) its cell surface. In general, the term “antigen-presenting cell” can be any cell that accomplishes the goal of the invention by aiding the enhancement of an immune response (i.e., from the T-cell or -B-cell arms of the immune system) against an antigen or antigenic composition. Such cells can be defined by those of skill in the art, using methods disclosed herein and in the art. As is understood by one of ordinary skill in the art, a cell that displays or presents an antigen normally or preferentially within or bound to a class I or class II major histocompatibility molecule or complex to an immune cell is an “antigen-presenting cell.” In some cases, the immune cell to which an antigen-presenting cell displays or presents an antigen to is a CD4⁺ TH cell. Additional molecules expressed on the APC or other immune cells may aid or improve the enhancement of an immune response. Secreted or soluble molecules, such as for example, cytokines and adjuvants, may also aid or enhance the immune response against an antigen. Such molecules are well known to one of skill in the art, and various examples are described herein. In specific embodiments, an antigen-presenting cell comprises a dendritic cell.

The term “dendritic cell” (DC) is an antigen presenting cell existing in vivo, in vitro, ex vivo, or in a host or subject, or which can be derived from a hematopoietic stem cell or a monocyte. Dendritic cells and their precursors can be isolated from a variety of lymphoid organs, e.g., spleen, lymph nodes, as well as from bone marrow and peripheral blood. The DC has a characteristic morphology with thin sheets (lamellipodia) extending in multiple directions away from the dendritic cell body. Typically, dendritic cells express high levels of MHC and costimulatory (e.g., B7-1 and B7-2) molecules. Dendritic cells can induce antigen specific differentiation of T cells in vitro, and are able to initiate primary T cell responses in vitro and in vivo.

The term “dendritic cell-based vaccine” as used herein is defined as a vaccine that comprises an ex vivo dendritic cell comprising SHP-1 modulatory agent (and/or antigen) or a vaccine that comprises a SHP-1 modulatory agent (and/or antigen) without a dendritic cell, but upon in vivo delivery the agent is uptaken by a dendritic cell.

As used herein the term “effective amount” is defined as an amount of the SHP-1 modulatory agent, such as a SHP-1 inhibitory agent, (or such as the dendritic cell vaccine transduced with an adenoviral vector containing a shRNA sequence that inhibits SHP-1 function or a combination of the dendritic cell vaccine transduced with an adenoviral vector containing a mRNA sequence that inhibits SHP-1 function and an antigen) that is sufficient to detectably inhibit growth or proliferation of a cell including a cancer.

The term “cancer” as used herein is defined as a hyperproliferation of cells whose unique trait—loss of normal control—results in unregulated growth, lack of differentiation, local tissue invasion, and/or metastasis. Examples include but are not limited to, breast cancer, prostate cancer, ovarian cancer, cervical cancer, skin cancer, pancreatic cancer, colorectal cancer, renal cancer and lung cancer.

The term “hyperproliferative disease” is defined as a disease that results from a hyperproliferation of cells. Exemplary hyperproliferative diseases include, but are not limited to, cancer or autoimmune diseases. Other hyperproliferative diseases may include vascular occlusion, restenosis, atherosclerosis, or inflammatory bowel disease, for example.

The term “therapeutically effective amount” as used herein is defined as the amount of a dendritic cell vaccine required to improve at least one symptom associated with a disease. For example, in the treatment of cancer, a dendritic cell vaccine that decreases, prevents, delays or arrests any symptom of the cancer is therapeutically effective. A therapeutically effective amount of a dendritic cell vaccine is not required to cure a disease but will provide a treatment for a disease. A dendritic cell vaccine is to be administered in a therapeutically effective amount if the amount administered is physiologically significant. A dendritic cell vaccine is physiologically significant if its presence results in technical change in the physiology of a recipient individual.

The term “sample” as used herein indicates a patient sample containing at least one cancer cell, including at least one tumor cell. Tissue or cell samples can be removed from almost any part of the body. The most appropriate method for obtaining a sample depends on the type of cancer that is suspected or diagnosed. Biopsy methods include needle, endoscopic, and excisional, for example.

As used herein, the terms “treatment”, “treat”, “treated”, or “treating” refer to prophylaxis and/or therapy. When used with respect to cancer, for example, the term refers to a prophylactic or remediation treatment that kills cancer cells, induces apoptosis in cancer cells, reduces the growth rate of cancer cells, reduces the incidence or number of metastases, reduces tumor size, inhibits tumor growth, reduces the blood supply to a tumor or cancer cells, promotes an immune response against cancer cells or a tumor, prevents or inhibits the progression of cancer, and/or increases the lifespan of a subject with cancer. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse.

As used herein, the term “vaccine” refers to a formulation which contains the composition of the present invention and which is in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into a subject, the vaccine is able to provoke an immune response including, but not limited to, the production of antibodies, cytokines and/or other cellular responses.

As used herein, the terms “SHP-1 modulating agent”, “SHP-1 modulator” or “SHP-1 modulatory agent” refers to a formulation that contains a shRNA or mRNA sequence of the present invention that is capable of stimulating or inhibiting SHP-1 function. For example, a “SHP-1 inhibitory agent” refers to a formulation that contains a shRNA or mRNA sequence of the present invention that is capable of inhibiting SHP-1 function and a “SHP-1 stimulatory agent” refers to a formulation that contains a shRNA or mRNA sequence of the present invention that is capable of stimulating SHP-1 function.

Any of the methods described herein may be implemented using therapeutic compositions of the invention and vice versa. It is contemplated that any embodiment discussed with respect to an aspect of the invention may be implemented or employed in the context of other aspects of the invention.

### II. Enhancement of an Immune Response

Embodiments of the present invention includes compositions and methods for modulating an immune response in an individual comprising the steps of contacting one or more lymphocytes with a dendritic cell (DC) or DC modifying vaccine (such as a DNA construct or RNA construct) and a vector containing a shRNA or mRNA that modulates SHP-1 function, and in some cases the vaccine also comprises an antigenic composition. In specific embodiments, the shRNA comprises at least as part of its sequence a sequence that targets SHP-1 (exemplary shRNAs include SEQ ID NO: 11 and SEQ ID NO: 12), or an immunologically functional equivalent thereof. In specific embodiments, the SHP-1 mutant construct comprises at least as part of its sequence a sequence that targets SHP-1 (exemplary SHP-1 mutant constructs include SEQ ID NO:7, SEQ ID NO:8, and SEQ ID NO:9), or an immunologically functional equivalent thereof. As used herein, an “antigenic composition” may comprise an antigen (e.g., a peptide or polypeptide carbohydrate, lipid, or polynucleotide), a nucleic acid encoding an antigen (e.g., an antigen expression vector), or a cell expressing or presenting an antigen. In particular embodiments, the shRNA targets SHP-1, and an exemplary SHP-1 polynucleotide sequence is provided in SEQ ID NO:10 (GenBank® Accession No. BC012660), SEQ ID NO:14 (GenBank® Accession No. NM_{—}013545), SEQ ID NO:15 (GenBank® Accession No. NM_{—}001077705), for mouse SHP-1; and SEQ ID NO:16 (GenBank® Accession No. NM_{—}002831), SEQ ID NO:17 (GenBank® Accession No. NM_{—}080549), SEQ ID NO:18 (GenBank® Accession No. NM_{—}080548), and SEQ ID NO:19 (GenBank® Accession No. BC002523) for human SHP-1. In certain cases, the SHP-1 modulatory agent inhibits SHP-1 by inhibiting the expression of the SHP-1 gene product.

One of skill in the art recognizes that the SHP-1 target sequence provided herein allows one to employ any sequence for the inhibitory agent. In particular, one may utilize the provided SHP-1 target sequence to identify sequences for use as one or more inhibitory RNA molecules, including without limitation shRNA, siRNA, and RNAi molecules, for example. Particular subsequences of SHP-1 can be selected by the person of ordinary skill in the art for the inhibitory RNA molecule of interest, using, for example, commercially available sources to identify useful sequences (e.g., custom RNA interference services and manufacturing from Invitrogen (Carlsbad, Calif.)). The sequence of the SHP-1 modulatory agent may be of any length, but in specific embodiments the length is at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 60, at least 70, at least 75, at least 100, at least 125, at least 150, at least 175, at least 200, at least 225, at least 250, at least 275, at least 300, at least 325, at least 350, at least 375, at least 400, and least 425, at least 450, at least 475, at least 500, at least 525, at least 550, at least 575, at least 600, at least 625, at least 650, at least 675, at least 700, at least 750, at least 1000, at least 1500, or at least 2000 nucleotides in length. In particular embodiments, the length of the SHP-1 modulatory agent is no more than 2000, no more than 1500, no more than 1000, no more than 750, no more than 500, no more than 250, no more than 100, no more than 75, no more than 50, no more than 40, no more than 35, no more than 30, no more than 25, no more than 24, no more than 23, no more than 22, no more than 21, no more than 20, no more than 19, no more than 18, no more than 17, no more than 16, no more than 15, no more than 14, no more than 13, no more than 12, no more than 11, or no more than 10 nucleotides in length. In certain cases, the length of the SHP-1 modulatory agent is within a range of nucleotides, including, for example, 20-25 nucleotides, 20-23 nucleotides, 20-22 nucleotides, 20-21 nucleotides, 10-22 nucleotides, 15-22 nucleotides, 17-22 nucleotides, 18-22 nucleotides, 19-22 nucleotides, 25-55 nucleotides, 30-60 nucleotides, 35-55 nucleotides, 40-55 nucleotides, 45-55 nucleotides, 50-55 nucleotides, and so forth. In certain embodiments, the SHP-1 modulatory agent is, or includes, a subsequence of consecutive nucleotides from a SHP-1 nucleotide sequence described herein. In certain cases, the SHP-1 modulatory agent includes one or more filler sequences that do not correspond to the target SHP-1 sequence (e.g., a non-SHP-1 sequence adjacent to a SHP-1 subsequence or between two SHP-1 subsequences).

In other embodiments, the antigenic composition is in a mixture that comprises an additional immunostimulatory agent or nucleic acids encoding such an agent. Immunostimulatory agents include but are not limited to an additional antigen, an immunomodulator, an antigen presenting cell or an adjuvant, for example.

The enhanced immune response is an active immune response, in specific embodiments. Alternatively, the response may be part of an adoptive immunotherapy approach in which lymphocyte(s) are obtained with from an animal (e.g., a patient), then pulsed with composition comprising an antigenic composition and with a SHP-1 modulatory agent. In this embodiment, the antigenic composition may comprise an additional immunostimulatory agent or a nucleic acid encoding such an agent. The lymphocyte(s) may be obtained from the blood of the subject, or alternatively from tumor tissue to obtain tumor infiltrating lymphocyte(s). In certain preferred embodiments, the lymphocyte(s) are peripheral blood lymphocyte(s). In a preferred embodiment, the lymphocyte(s) be administered to the same or different animal (e.g., same or different donors). In a preferred embodiment, the animal (e.g., a patient) has or is suspected of having a cancer, such as for example, prostate cancer or breast cancer. In other embodiments the method of enhancing the immune response is practiced in conjunction with a cancer therapy, such as for example, a cancer vaccine therapy.

### III. SHP-1 Modulatory Agents

In the present invention, a SHP-1 modulatory agent is employed to enhance a dendritic cell-based vaccine. In some cases, such as cancer and against a disease caused by a pathogen, a SHP-1 inhibitory agent is utilized, whereas in other cases, such as autoimmune disorders, a SHP-1 stimulatory agent is utilized.

A. SHP-1 Inhibitory Agents

In some embodiments of the invention, a SHP-1 inhibitory agent is utilized to enhance a dendritic cell-based vaccine, such as for a cancer vaccine or pathogenic disease vaccine. The SHP-1 inhibitory agent may be of any kind, although in certain embodiments the agent is a nucleic acid molecule, a protein, a peptide, or a small molecule, for example. The inhibitory agent may be a RNA or a DNA, or a mixture thereof, including a dsDNA, ssDNA, ssRNA, or dsRNA. In certain embodiments of the invention, the inhibitory agent encompasses RNAi compositions, shRNA compositions, siRNA compositions, dsRNA compositions, and so forth. An exemplary small molecule that may be employed as the SHP-1 inhibitory agent includes sodium stibogluconate (GlaxoSmithKline, UK). In specific cases, a dominant negative mutant of SHP-1 is used, for example to overcome its normal function by competing for substrate without performing its catalytic activity.

B. SHP-1 Stimulatory Agents

In certain embodiments of the invention, a SHP-1 stimulatory agent is utilized in a vaccine, such as for a vaccine for one or more autoimmune diseases. The SHP-1 stimulatory agent may be of any kind, although in certain embodiments the agent is a wild-type SHP-1 to overexpress SHP-1 catalytic activity or a constitutively active SHP-1 to augment endogenous SHP-1 function by providing continuous SHP-1 catalytic activity without having normal regulatory mechanisms. In particular aspects, the stimulatory agent is a polypeptide, including one encoded by a nucleic acid of the invention.

### IV. Antigens

The present invention, in certain embodiments, employs an antigen that causes an immune response to a particular medical condition. The antigen may comprise a peptide or polypeptide, in certain embodiments. In particular, for cancer a tumor antigen is employed in the invention in conjunction with the SHP-1 modulatory agent. In particular aspects, the antigen may be of any length, although in certain cases the antigen is at least 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, or more amino acids in length. The antigen may comprise a peptide between 7 and 12 amino acids, 7 and 15 amino acids, 8 and 12 amino acids, 8 and 15 amino acids, 9 and 12 amino acids, or 9 and 15 amino acids in length, for example.

Tumor antigen is a substance produced in tumor cells that triggers an immune response in the host. Tumor antigens are useful in identifying tumor cells and are potential candidates for use in cancer therapy. Normal proteins in the body are not antigenic because of self-tolerance, a process in which self-reacting cytotoxic T lymphocytes (CTLs) and autoantibody-producing B lymphocytes are culled in the thymus. Thus any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation.

Any protein produced in a a tumor cell that has an abnormal structure due to mutation can act as a tumor antigen. Such abnormal proteins are produced due to mutation of the concerned gene. Mutation of protooncogenes and tumor suppressors that lead to abnormal protein production are the cause of the tumor and thus such abnormal proteins are called tumor-specific antigens. Examples of tumor-specific antigens include the abnormal products of ras and p53 genes. In contrast, mutation of other genes unrelated to the tumor formation may lead to synthesis of abnormal proteins that are called tumor-associated antigens. Non limiting examples of tumor antigens include the following: Alphafetoprotein (AFP), Carcinoembryonic antigen (CEA), CA-125 (ovarian cancer), MUC-1 (breast cancer), epithelial tumor antigen (ETA) (breast cancer), tyrosinase (malignant melanoma, normally present in minute quantities; greatly elevated levels in melanoma), melanoma-associated antigen (MAGE) (malignant melanoma, also normally present in the testis), abnormal products of ras and p53 (various tumors), beta subunit of hCG, prostate specific antigen, beta 2 microglobulin, CA19-9, CA15-3, chromagram A, thyroglobulin, TA-90, Brain-associated small-cell lung cancer antigen (BASCA), colon cancer antigen 1 gene (SDCCAG1), human CO17-1A/GA733 colon cancer antigen, urinary bladder cancer antigens (CYFRA 21-1, NMP22), cancer-testis antigen (NY-ESO-1), prostate specific membrane antigen (PSMA), prostatic alkaline phosphatase (PAP), six transmembrane epithelial antigen of the prostate (STEAP), prostate stem cell antigen (PSCA), Human telomerase reverse transcriptase (hTERT), tyrosinase-related protein (TRP-1 and TRP-2), human melanoma antigens (MART-1, gp100, tyrosinase), Human Epidermal Growth Factor Receptor 2 (HER2), breast cancer antigens (NY-BR-1, NY-BR-62, NY-BR-75, NY-BR-85, NY-BR-96, D52).

### V. Methods for Treating a Disease

The present invention also encompasses methods of treatment and/or prevention of a disease caused by pathogenic microorganisms, autoimmune disorder and/or a hyperproliferative disease.

Diseases that may be treated or prevented by use of the present invention include diseases caused by viruses, bacteria, yeast, parasites, protozoa, cancer cells and the like. The pharmaceutical composition of the present invention (transduced DCs, expression vector, expression construct, etc.) of the present invention may be used as a generalized immune enhancer (DC activating composition or system) and as such has utility in treating diseases. Exemplary diseases that can be treated and/or prevented utilizing the pharmaceutical composition of the present invention include, but are not limited to infections of viral etiology such as HIV, influenza, Herpes, viral hepatitis, Epstein Bar, polio, viral encephalitis, measles, chicken pox, Papilloma virus etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc.

Preneoplastic or hyperplastic states that may be treated or prevented using the pharmaceutical composition of the present invention (transduced DCs, expression vector, expression construct, etc.) of the present invention include but are not limited to preneoplastic or hyperplastic states such as colon polyps, Crohn's disease, ulcerative colitis, breast lesions and the like.

Cancers that may be treated using the composition of the present invention of the present invention include, but are not limited to primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemias, uterine cancer, breast cancer, prostate cancer, ovarian cancer, pancreatic cancer, colon cancer, multiple myeloma, neuroblastoma, NPC, bladder cancer, cervical cancer and the like.

Other hyperproliferative diseases that may be treated using DC activation system of the present invention include, but are not limited to rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

Autoimmune disorders that may be treated using the composition of the present invention include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, and extracorporeal circulation; viral, bacterial, fungal, parasitic, protozoal, and helminthic infections; and trauma.

In the method of treatment, the administration of the composition (expression construct, expression vector, fused protein, transduced cells, activated DCs, transduced and loaded DCs) of the invention may be for either “prophylactic” or “therapeutic” purpose. When provided prophylactically, the composition of the present invention is provided in advance of any symptom, although in particular embodiments the vaccine is provided following the onset of one or more symptoms to prevent further symptoms from developing or to prevent present symptoms from becoming worse. The prophylactic administration of composition serves to prevent or ameliorate any subsequent infection or disease. When provided therapeutically, the pharmaceutical composition is provided at or after the onset of a symptom of infection or disease. Thus, the present invention may be provided either prior to the anticipated exposure to a disease-causing agent or disease state or after the initiation of the infection or disease.

The term “unit dose” as it pertains to the inoculum refers to physically discrete units suitable as unitary dosages for mammals, each unit containing a predetermined quantity of pharmaceutical composition calculated to produce the desired immunogenic effect in association with the required diluent. The specifications for the novel unit dose of an inoculum of this invention are dictated by and are dependent upon the unique characteristics of the pharmaceutical composition and the particular immunologic effect to be achieved.

An effective amount of the composition would be the amount that achieves this selected result of enhancing the immune response, and such an amount could be determined as a matter of routine by a person skilled in the art. For example, an effective amount of for treating an immune system deficiency against cancer or pathogen could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

A. Genetic Based Therapies

Specifically, the present inventors intend to provide, to an individual or a cell, an expression construct that encompasses a SHP-1 modulatory agent. In specific embodiments, an expression construct capable of providing a co-stimulatory polypeptide, such as CD40 to the cell, such as an antigen-presenting cell and activating CD40, is provided. Particularly preferred expression vectors are viral vectors such as adenovirus, adeno-associated virus, herpes virus, vaccinia virus, lentivirus, and retrovirus. Also preferred is lysosomal-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

B. Cell based Therapy

Another therapy that is contemplated is the administration of transduced dendritic cell vaccines. The dendritic cells may be transduced in vitro. Formulation as a pharmaceutically acceptable composition is discussed above.

In cell based therapies, the transduced dendritic cells may be transfected with target antigen compositions, such as mRNA or DNA or peptides or proteins; pulsed with cell lysates, peptides, proteins or nucleic acids; or electrofused with cells. The cells, proteins, cell lysates, or nucleic acid may derive from cells, such as tumor cells or other pathogenic microorganism, for example, viruses, bacteria, protozoa, etc.

### VI. Nucleic Acid Vaccines

In certain embodiments of the invention, a nucleic acid vaccine is employed in the invention. In particular aspects, a nucleic acid vaccine comprising a SHP-1 modulatory agent is utilized. The SHP-1 modulatory agent itself may be a RNA or a DNA, which may be single-stranded or double-stranded. In some embodiments, the RNA or DNA may encode a peptide or protein that is the SHP-1 modulatory agent. In certain aspects, the nucleic acid vaccine comprises a vector harboring a nucleic acid sequence that is the SHP-1 modulatory agent or encodes a RNA, peptide, or protein that is the SHP-1 modulatory agent. The vector may be of any kind, although in specific embodiments it is a viral vector, for example an adenoviral vector. In certain aspects of the invention, the nucleic acid sequence that is the SHP-1 modulatory agent or encodes a RNA, peptide, or protein that is the SHP-1 modulatory agent is regulated by a regulatory sequence, such as a promoter. In certain cases, the regulatory sequence is active in dendritic cells, for example CD11c. An exemplary CD11c regulatory sequence is provided in GenBank® Accession No. DQ658851 (SEQ ID NO:20) and SEQ ID NO:21 (a region upstream from the coding region; from GenBank® AC026471). In specific cases, the nucleic acid vaccine alternatively or additionally comprises antigenic nucleic acid sequence, such as nucleic acid sequence that encodes an antigen, including a tumor antigen, and for example an antigen that comprises a peptide. In particular embodiments, there is a vector that includes the SHP-1 modulatory agent nucleic acid sequence and also includes an antigen nucleic acid sequence. In further particular embodiments, one or both of the antigen and SHP-1 nucleic acid sequences are under the regulation of a dendritic cell-specific regulatory region, such as a CD11c regulatory region.

Therefore, in particular embodiments, an individual that is in need of a vaccine is provided a nucleic acid vaccine harboring a SHP-1 modulatory agent. Although in some embodiments of this invention nucleic acid(s) for the SHP-1 modulatory agent and/or antigen are delivered to a dendritic cell ex vivo, in certain cases, the vaccine is delivered to an individual without being present in a dendritic cell. In some cases, the nucleic acids are uptaken by dendritic cells in the body of the individual for use against the medical condition being treated.

### VII. Combination Treatments

In specific embodiments in which the dendritic cell (DC) vaccine of the present invention are employed, it may be desirable to combine the DC vaccine of the present invention with other agents effective in the treatment of the medical condition. In the case of hyperproliferative disease, for example, anti-cancer agent(s) may be employed, for example. In the case of pathogenic disease, antibiotics or antivirals may be employed, for example. In the case of autoimmune diseases, corticosteroid drugs, non-steroidal anti-inflammatory drugs (NSAIDs) or immunosuppressant drugs such as cyclophosphamide, methotrexate or azathioprine may be employed, for example. An example of antibiotics includes penicillins such as penicillin and amoxicillin; cephalosporins such as cephalexin; macrolides such as erythromycin, clarithromycin, and azithromycin; fluoroquinolones such as ciprofloxacin, levofloxacin, and ofloxacin; sulfonamides such as co-trimoxazole and trimethoprim; tetracyclines such as tetracycline and doxycycline; and aminoglycosides such as gentamicin and tobramycin. Exemplary antivirals include seltamivir; zanamivir; amantadine; rimantadine; trifluridine, famcyclovir, valacyclovir, acyclovir, vidarabine, gancyclovir, valgancyclovir, cidofovir, foscarnet, fomivirsen, zidovudine, didanosine, lamivudine, zalcibabine, abacavir, nucleoside reverse transcriptase inhibitors, nonnucleoside reverse transcriptase inhibitors, protease inhibitors, and so forth.

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, and/or increasing the lifespan of a subject with cancer. Anti-cancer agents include biological agents (biotherapy), chemotherapy agents, immunotherapy agents, surgery, and radiotherapy agents. More generally, these other compositions would be provided in a combined amount effective to kill or inhibit proliferation of the cell. This process may involve contacting the cells with the antibodies of the present invention and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the DC vaccine and the other includes the second agent(s).

Alternatively, the DC vaccine of the present invention may precede or follow the other anti-cancer agent treatment by intervals ranging from minutes to weeks. In embodiments where the other anti-cancer agent and DC vaccine are applied separately to the individual or a cell thereof, one would generally ensure that a significant period of time did not expire between the time of each delivery, such that the agent and DC vaccine would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly, however, where several days (2, 3, 4, 5, 6 or 7, for example) to several weeks (1, 2, 3, 4, 5, 6, 7 or 8, for example) lapse between the respective administrations.

A. Chemotherapy

Cancer therapies also include a variety of chemical-based treatments. Some examples of chemotherapeutic agents include antibiotic chemotherapeutics such as Doxorubicin, Daunorubicin, Adriamycin, Mitomycin (also known as mutamycin and/or mitomycin-C), Actinomycin D (Dactinomycin), Bleomycin, Plicomycin, plant alkaloids such as Taxol, Vincristine, Vinblastine, miscellaneous agents such as Cisplatin (CDDP), etoposide (VP16), Tumor Necrosis Factor, and alkylating agents such as, Carmustine, Melphalan (also known as alkeran, L-phenylalanine mustard, phenylalanine mustard, L-PAM, or L-sarcolysin, is a phenylalanine derivative of nitrogen mustard), Cyclophosphamide, Chlorambucil, Busulfan (also known as myleran), Lomustine.

Some examples of other agents include, but are not limited to, Carboplatin, Procarbazine, Mechlorethamine, Camptothecin, Ifosfamide, Nitrosurea, Etoposide (VP16), Tamoxifen, Raloxifene, Toremifene, Idoxifene, Droloxifene, TAT-59, Zindoxifene, Trioxifene, ICI-182,780, EM-800, Estrogen Receptor Binding Agents, Gemcitabien, Navelbine, Farnesyl-protein transferase inhibitors, Transplatinum, 5-Fluorouracil, hydrogen peroxide, and Methotrexate, Temazolomide (an aqueous form of DTIC), Mylotarg, Dolastatin-10, Bryostatin, or any analog or derivative variant of the foregoing.

B. Radiotherapeutic Agents

Radiotherapeutic agents and factors include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage in DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

C. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

D. Gene Therapy

In yet another embodiment, gene therapy in conjunction with the combination therapy using the DC vaccines described in the invention are contemplated. A variety of genes that may be targeted for gene therapy of some form in combination with the present invention include, but are not limited to growth factors, receptor tyrosine kinases, non-receptor tyrosine kinases, SER/THR protein kinases, cell surface proteins, cell signaling proteins, guanine nucleotide exchangers and binding proteins, or nuclear proteins, or nuclear transcription factors.

E. Other Agents

It is contemplated that other agents may be used in combination with the present invention to improve the therapeutic efficacy of treatment. One form of therapy for use in conjunction with chemotherapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention. The use of hormones may be employed in the treatment of certain cancers such as breast, prostate, ovarian, or cervical cancer to lower the level or block the effects of certain hormones such as testosterone or estrogen and this often reduces the risk of metastases.

Adjuvant therapy may also be used in conjunction with the present invention. The use of adjuvants or immunomodulatory agents include, but are not limited to tumor necrosis factor; interferon alpha, beta, and gamma; IL-2 and other cytokines; F42K and other cytokine analogs; or MIP-1, MIP-1beta, MCP-1, RANTES, and other chemokines.

### VIII. Vaccines

It is contemplated that vaccines that are used to treat cancer or pathogens may be used in combination with the present invention to improve the therapeutic efficacy of the treatment. Such vaccines include dendritic cell vaccines. Yet further, one skilled in the art realizes that dendritic cell vaccination comprises dendritic cells that are pulsed with a peptide, in some embodiments, or antigen and the pulsed dendritic cells are administered to the patient. In particular, the dendritic cell comprises a SHP-1 modulatory agent. In alternative embodiments, the vaccine is one that is not provided in a cell.

In particular embodiments, the present invention concerns an immunogenic composition comprising a SHP-1 modulatory agent. In specific cases, an immunogenic composition induces an immune response to an antigen in a cell, tissue or animal (e.g., a human). In some embodiments, the immunogenic composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

A vaccine of the present invention may vary in its composition of proteinaceous, nucleic acid and/or cellular components. In a non-limiting example, acid nucleic encoding the SHP-1 modulatory agent and, optionally, an antigen might also be formulated with a proteinaceous adjuvant. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected,” “transduced,” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

### III. Autoimmune Diseases

The dendritic cell vaccines of the method and composition of the present invention may be administered to a subject to prevent or treat an immune disorder. In this embodiment, the dendritic cells comprise an agent that enhances SHP-1 expression and/or activity. Although in certain embodiments the agent may comprise a vector that overexpresses SHP-1, in alternative embodiments the agent comprises a constitutively active SHP-1 shRNA sequence (SEQ ID NO: 9) that induces immune tolerance.

Such disorders may include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, and extracorporeal circulation; viral, bacterial, fungal, parasitic, protozoal, and helminthic infections; and trauma.

“Systemic lupus erythematosus (SLE)” as used herein refers to an autoimmune disorder in which autoantibodies are found and thought to be important in etiology and pathogenesis. SLE can be grouped with those diseases that commonly have autoantibodies present but for whom a central role of autoantibody in pathogenesis leading to clinical expression has yet to be fully established or accepted. The most common antigens in SLE and closely related disorders include: Ro/SSA, La/SSB, nRNP and Sm. It is contemplated that autoantigens for SLE comprise alternatively spliced isoform-specific regions of any of the above-mentioned proteins.

“Insulin-dependent diabetes mellitus (IDDM)” as used herein refers to an autoimmune disease that results from the destruction of the insulin-secreting beta-cells of the pancreas. Antibodies to two glutamate acid decarboxylase isoforms, insulin, carboxypeptidase H, ICA 516 and 64 kD integral membrane proteins, hsp65, and several secretory granule protein have been found in the sera of diabetic and prediabetic individuals. Peripheral blood T cells from a majority of persons newly diagnosed with IDDM respond to a variety of insulin-secretory granule antigens. It is contemplated that autoantigens for IDDM comprise alternatively spliced isoform-specific regions of any of the above-mentioned proteins.

Patients with a rare but severe neurological disease, “Stiff Man Syndrome (SMS)”, have autoantibodies to GABA-ergic neurons. Glutamic acid decarboxylase (GAD), the enzyme that synthesizes GABA from glutamic acid, was found to be the predominant autoantigen. It is contemplated that autoantigens for SMS comprise alternatively spliced isoform-specific regions of GAD or any other SMS-associated proteins.

“MS” is an immune-mediated disorder characterized pathologically by perivenular white matter infiltrates comprised of macrophages and mononuclear cells (inflammation), and destruction of the myelin sheaths that insulate nerve fibers (demyelination). A key role of myelin oligodendrocyte glycoprotein (MOG) is in plaque formation. It is contemplated that alternatively spliced isoforms MOG may be MS autoantigens.

### IV. shRNAs

A small hairpin RNA or short hairpin RNA (shRNA) is a sequence of RNA that makes a tight hairpin turn that can be used to silence gene expression via RNA interference, although in alternative embodiments the RNA interference that is employed comprises dsRNA or siRNA. shRNA uses a vector introduced into cells and utilizes a promoter, such as the U6 promoter, to ensure that the shRNA is always expressed. This vector is usually passed on to daughter cells, allowing the gene silencing to be inherited, unless, for example a vector is not integrated, such as an adenoviral vector. The shRNA hairpin structure is cleaved by the cellular machinery into shRNA, which is then bound to the RNA-induced silencing complex (RISC). This complex binds to and cleaves mRNAs that match the shRNA that is bound to it.

shRNA is transcribed by RNA polymerase III. shRNA production in a mammalian cell can sometimes cause the cell to mount an interferon response as the cell seeks to defend itself from what it perceives as viral attack. This problem is not observed in miRNA, which is transcribed by RNA polymerase II (the same polymerase used to transcribe mRNA).

The present invention provides a small hairpin RNA that modulates (e.g., stimulates, partially inhibits or completely inhibits) expression of a gene of interest (i.e., SHP-1 function). A shRNA can be provided shRNA transcribed from a transcriptional cassette in a DNA plasmid. The shRNA may also be chemically synthesized. The shRNA can be administered alone or co-administered (i.e., concurrently or consecutively) with conventional agents used to suppress an immune response or induce a pro-inflammatory immune response.

In one aspect, the interfering RNA is an shRNA molecule that is capable of inhibiting SHP-1 function. In some embodiments, the shRNA molecules are about 15 to 60 nucleotides in length. The synthesized or transcribed shRNA can have 3′ overhangs of about 1-4 nucleotides, preferably of about 2-3 nucleotides, and 5′ phosphate termini. In some embodiments, the shRNA lacks terminal phosphates.

In certain embodiments, the shRNA molecules of the present invention are chemically modified as described herein. In certain preferred embodiments, the shRNA molecules of the present invention comprise less than about 20% modified nucleotides. The modified shRNA molecule is notably less immunostimulatory than a corresponding unmodified shRNA sequence and retains full RNAi activity against the target sequence, in certain embodiments. Preferably, the modified shRNA contains at least one 2′OMe purine or pyrimidine nucleotide such as a 2′OMe-guanosine, 2′OMe-uridine, 2′OMe-adenosine, and/or 2′OMe-cytosine nucleotide.

Importantly, shRNA molecules that are immunostimulatory can be modified to decrease their immunostimulatory properties without having a negative impact on RNAi activity. For example, an immunostimulatory shRNA can be modified by replacing one or more nucleotides in the sense and/or antisense strand with a modified nucleotide, thereby generating a modified shRNA with reduced immunostimulatory properties that is still capable of silencing expression of the target sequence.

It is also preferred that the modified shRNA comprises less than about 20% modified nucleotides (e.g., less than about 20%, 19%, 18%, 17%, 16%, 15%, 14%, 13%, 12%, 11%, 10%, 9%, 8%, 7%, 6%, 5%, 4%, 3%, 2%, or 1% modified nucleotides) or between about 1%-20% modified nucleotides (e.g., between about 1%-20%, 2%-20%, 3%-20%, 4%-20%, 5%-20%, 6%-20%, 7%-20%, 8%-20%, 9%-20%, 10%-20%, 11%-20%, 12%-20%, 13%-20%, 14%-20%, 15-20%, 16%-20%, 17%-20%, 18%-20%, or 19%-20% modified nucleotides). However, when one or both strands of the shRNA are selectively modified at uridine and/or guanosine nucleotides, the resulting modified shRNA molecule can comprise less than about 25% modified nucleotides (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% modified nucleotides) or between about 1%-25% modified nucleotides (e.g., between about 1%-25%, 2%-25%, 3%-25%, 4%-25%, 5%-25%, 6%-25%, 7%-25%, 8%-25%, 9%-25%, 10%-25%, 11%-25%, 12%-25%, 13%-25%, 14%-25%, 15-25%, 16%-25%, 17%-25%, 18%-25%, 19%-25%, 20%-25%, 21%-25%, 22%-25%, 23%-25%, or 24%-25% modified nucleotides).

In specific embodiments, the shRNA that is employed may be of any length so long as it effectively knocks down SHP-1 expression. In some cases, the length is 21 bases, but an exemplary range is 20-30 bases, in certain cases. In some cases, identity between the template and the corresponding strand of the shRNA is 100%, although in other cases there may be some mismatch, including at least 99%, at least 97%, at least 95%, at least 90%, at least 85%, and so on. In the case of mutant mRNA, such as dominant negative dn-SHP-1 or constitutively active ca-SHP-1, these can be 99.99% similar or much less, having no lower limit.

A. Selection of shRNA Sequences

Once a potential shRNA sequence has been identified, the sequence can be analyzed using a variety of criteria known in the art. For example, the shRNA sequences may be analyzed by a rational design algorithm to identify sequences that have G/C content of about 25% to about 60% G/C. shRNA design tools that incorporate algorithms that assign suitable values of this and other features and are useful for selection of shRNA can be found on the world wide web. One of skill in the art will appreciate that sequences with one or more of the foregoing characteristics may be selected for further analysis and testing as potential shRNA sequences. shRNA sequences complementary to the shRNA target sites may also be designed.

Once a potential shRNA sequence has been identified, the sequence can be analyzed for the presence of any immunostimulatory properties, e.g., using an in vitro cytokine assay or an in vivo animal model. Motifs in the sense and/or antisense strand of the shRNA sequence such as GU-rich motifs can also provide an indication of whether the sequence may be immunostimulatory. Once an shRNA molecule is found to be immunostimulatory, it can then be modified to decrease or increase its immunostimulatory properties as described herein. As a non-limiting example, an shRNA sequence can be contacted with a mammalian responder cell under conditions such that the cell produces a detectable immune response to determine whether the shRNA is an immunostimulatory or a non-immunostimulatory shRNA. The mammalian responder cell may be from a naive mammal (i.e., a mammal that has not previously been in contact with the gene product of the shRNA sequence). The mammalian responder cell may be, e.g., a peripheral blood mononuclear cell (PBMC), a macrophage, and the like. The detectable immune response may comprise production of a cytokine or growth factor such as, e.g., TNF-α, TNF-β, IFN-α, IFN-γ, IL-6, IL-12, or a combination thereof. An shRNA molecule identified as being immunostimulatory can then be modified to increase or decrease its immunostimulatory properties by replacing at least one of the nucleotides on the sense and/or antisense strand with modified nucleotides. For example, less than about 20% of the nucleotides in the shRNA duplex can be replaced with modified nucleotides such as 2′OMe nucleotides. The modified shRNA can then be contacted with a mammalian responder cell as described above to confirm that its immunostimulatory properties have been enhanced or reduced.

A non-limiting example of an in vivo model for detecting an immune response includes an in vivo mouse cytokine induction assay that can be performed as follows: (1) shRNA can be administered by standard intravenous injection in the lateral tail vein; (2) blood can be collected by cardiac puncture about 6 hours after administration and processed as plasma for cytokine analysis; and (3) cytokines can be quantified using sandwich ELISA kits according to the manufacturers' instructions (e.g., mouse and human IFN-.alpha. (PBL Biomedical; Piscataway, N.J.); human IL-6 and TNF-αc (eBioscience; San Diego, Calif.); and mouse IL-6, TNF-.alpha., and IFN-γ. (BD Biosciences; San Diego, Calif.)).

B. Generating shRNA

shRNA molecules can be provided as transcribed from a transcriptional cassette in a DNA plasmid. The shRNA sequences may have overhangs (e.g., 3′ or 5′ overhangs as described in Elbashir et al., Genes Dev., 15:188 (2001) or Nykanen et al., Cell, 107:309 (2001), or may lack overhangs (i.e., have blunt ends).

An RNA population can be used to provide long precursor RNAs, or long precursor RNAs that have substantial or complete identity to a selected target sequence can be used to make the shRNA. The RNAs can be isolated from cells or tissue, synthesized, and/or cloned according to methods well known to those of skill in the art. The RNA can be a mixed population (obtained from cells or tissue, transcribed from cDNA, subtracted, selected, etc.), or can represent a single target sequence. RNA can be naturally occurring (e.g., isolated from tissue or cell samples), synthesized in vitro (e.g., using T7 or SP6 polymerase and PCR products or a cloned cDNA), or chemically synthesized.

To form a long dsRNA, for synthetic RNAs, the complement is also transcribed in vitro and hybridized to form a dsRNA. If a naturally occurring RNA population is used, the RNA complements are also provided (e.g., to form dsRNA for digestion by E. coli RNAse III or Dicer), e.g., by transcribing cDNAs corresponding to the RNA population, or by using RNA polymerases. The precursor RNAs are then hybridized to form double stranded RNAs for digestion. The dsRNAs can be directly administered to a subject or can be digested in vitro prior to administration.

Alternatively, one or more DNA plasmids encoding one or more shRNA templates are used to provide shRNA. shRNA can be transcribed as sequences that automatically fold into hairpin loops from DNA templates in plasmids having RNA polymerase III transcriptional units, for example, based on the naturally occurring transcription units for small nuclear RNA U6 or human RNase P RNA H1 (see, Brummelkamp et al., Science, 296:550 (2002); Donze et al., Nucleic Acids Res., 30:e46 (2002); Paddison et al., Genes Dev., 16:948 (2002); Yu et al., Proc. Natl. Acad. Sci. USA, 99:6047 (2002); Lee et al., Nat. Biotech., 20:500 (2002); Miyagishi et al., Nat. Biotech., 20:497 (2002); Paul et al., Nat. Biotech., 20:505 (2002); and Sui et al., Proc. Natl. Acad. Sci. USA, 99:5515 (2002)). Typically, a transcriptional unit or cassette will contain an RNA transcript promoter sequence, such as an H1-RNA or a U6 promoter, operably linked to a template for transcription of a desired shRNA sequence and a termination sequence, comprised of 2-3 uridine residues and a polythymidine (T5) sequence (polyadenylation signal) (Brummelkamp et al., supra). The selected promoter can provide for constitutive or inducible transcription. Compositions and methods for DNA-directed transcription of RNA interference molecules is described in detail in U.S. Pat. No. 6,573,099. The transcriptional unit is incorporated into a plasmid or DNA vector from which the interfering RNA is transcribed. Plasmids suitable for in vivo delivery of genetic material for therapeutic purposes are described in detail in U.S. Pat. Nos. 5,962,428 and 5,910,488. The selected plasmid can provide for transient or stable delivery of a target cell. It will be apparent to those of skill in the art that plasmids originally designed to express desired gene sequences can be modified to contain a transcriptional unit cassette for transcription of shRNA.

Methods for isolating RNA, synthesizing RNA, hybridizing nucleic acids, making and screening cDNA libraries, and performing PCR are well known in the art (see, e.g., Gubler and Hoffman, Gene 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994).

In some cases, shRNA are chemically synthesized. The oligonucleotides that comprise the shRNA molecule can be synthesized using any of a variety of techniques known in the art, such as those described in Usman et al., J. Am. Chem. Soc., 109:7845 (1987); Scaringe et al., Nuc. Acids Res., 18:5433 (1990); Wincott et al., Nuc. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol with a 2.5 min. coupling step for 2′-O-methylated nucleotides. Alternatively, syntheses at the 0.2 μmol scale can be performed on a 96-well plate synthesizer from Protogene (Palo Alto, Calif.). However, a larger or smaller scale of synthesis is also within the scope of the present invention. Suitable reagents for oligonucleotide synthesis, methods for RNA deprotection, and methods for RNA purification are known to those of skill in the art.

C. Modifying shRNA Sequences

In certain embodiments, the shRNA molecule can comprise one or more chemical modifications such as terminal cap moieties, phosphate backbone modifications, and the like. Examples of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, alpha.-nucleotides, modified base nucleotides, threo-pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3-aminopropyl phosphate, 6-aminohexyl phosphate, 1,2-aminododecyl phosphate, hydroxypropyl phosphate, 1,4-butanediol phosphate, 3′-phosphoramidate, 5′-phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties (see, e.g., U.S. Pat. No. 5,998,203; Beaucage et al., Tetrahedron, 49:1925 (1993)). Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions (see, e.g., Hunziker et al., Nucleic Acid Analogues: Synthesis and Properties, in Modern Synthetic Methods, VCH, 331-417 (1995); Mesmaeker et al., Novel Backbone Replacements for Oligonucleotides, in Carbohydrate Modifications in Antisense Research, ACS, 24-39 (1994)). Such chemical modifications can occur at the 5′-end and/or 3′-end of the strand of the shRNA. In specific embodiments, the SHP-1 constructs were modified by the addition of ten amino acid, N-terminal hemagglutin (HA), coding sequence as an epitope tag to facilitate subsequent detection and differentiation from endogenous SHP-1.

In some embodiments, the strand can comprise a 3′-terminal overhang having about 1 to about 4 (e.g., 1, 2, 3, or 4) 2′-deoxy ribonucleotides and/or any combination of modified and unmodified nucleotides. Additional examples of modified nucleotides and types of chemical modifications that can be introduced into the modified shRNA molecule are described, e.g., in UK Patent No. GB 2,397,818 B and U.S. Patent Publication Nos. 20040192626 and 20050282188.

The shRNA molecules described herein can optionally comprise one or more non-nucleotides in the strand of the shRNA. As used herein, the term “non-nucleotide” refers to any group or compound that can be incorporated into a nucleic acid chain in the place of one or more nucleotide units, including sugar and/or phosphate substitutions, and allows the remaining bases to exhibit their activity. The group or compound is abasic in that it does not contain a commonly recognized nucleotide base such as adenosine, guanine, cytosine, uracil, or thymine and therefore lacks a base at the 1′-position.

In other embodiments, chemical modification of the shRNA comprises attaching a conjugate to the shRNA molecule. The conjugate can be attached at the 5′ and/or 3′-end of the strand of the shRNA via a covalent attachment such as, e.g., a biodegradable linker. The conjugate can also be attached to the shRNA, e.g., through a carbamate group or other linking group (see, e.g., U.S. Patent Publication Nos. 20050074771, 20050043219, and 20050158727). In certain instances, the conjugate is a molecule that facilitates the delivery of the shRNA into a cell. Examples of conjugate molecules suitable for attachment to an shRNA include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 20030130186, 20040110296, and 20040249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 20050119470 and 20050107325. Yet other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 20050153337. Additional examples include the hydrophobic group, membrane active compound, cell penetrating compound, cell targeting signal, interaction modifier, and steric stabilizer conjugate molecules described in U.S. Patent Publication No. 20040167090. Further examples include the conjugate molecules described in U.S. Patent Publication No. 20050239739. The type of conjugate used and the extent of conjugation to the shRNA molecule can be evaluated for improved pharmacokinetic profiles, bioavailability, and/or stability of the shRNA. As such, one skilled in the art can screen shRNA molecules having various conjugates attached thereto to identify ones having improved properties using any of a variety of well-known in vitro cell culture or in vivo animal models.

### IX. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

A. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). DC vaccine components of the present invention may be a viral vector that encode one or more DC vaccine antigenic compositions or other components such as, for example, an immunomodulator or adjuvant. Non-limiting examples of virus vectors that may be used to deliver a nucleic acid of the present invention are described below

B. Adenoviral Vectors

In particular embodiments, an adenoviral expression vector is contemplated for the delivery of expression constructs. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear double stranded DNA, with a genome ranging from 30 to 35 kb in size (Reddy et al., 1998; Morrison et al., 1997; Chillon et al., 1999). An adenovirus expression vector according to the present invention comprises a genetically engineered form of the adenovirus. Advantages of adenoviral gene transfer include the ability to infect a wide variety of cell types, including non-dividing cells, a mid-sized genome, ease of manipulation, high infectivity and they can be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue-specific transforming construct that has been cloned therein.

The expression vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 hours. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1991; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing human primary cells.

### X. Nucleotide and Protein Sequences

The nucleotide and protein, polypeptide and peptide sequences for various genes have been previously disclosed, and may be found at computerized databases known to those of ordinary skill in the art. One such database is the National Center for Biotechnology Information's (NCBI) Genbank® and GenPept® databases available at the world wide web at the NCBI website. The coding regions for these known genes may be amplified and/or expressed using the techniques disclosed herein or by any technique that would be known to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

### XI. Kits of the Invention

Any of the compositions described herein may be comprised in a kit. In a non-limiting example, a SHP-1 modulatory agent and/or an antigen, or nucleic acids encompassing same, may be comprised in a kit. The reagents will be provided in suitable container means.

The kits may comprise a suitably aliquoted SHP-1 modulatory agent of the present invention. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the SHP-1 modulatory agent, antigen, and any other reagent containers in close confinement for commercial sale. Such containers may include injection or blow molded plastic containers into which the desired vials are retained.

### EXAMPLE

The following example is included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Methods and Reagents to Demonstrate that SHP-1 Inhibition is Effective in Enhancing Anti-Cancer Responses

Exemplary embodiments of methods and compositions demonstrating that SHP-1 inhibition is effective in enhancing anti-cancer responses are provided herein.

SHP-1 Specific shRNA

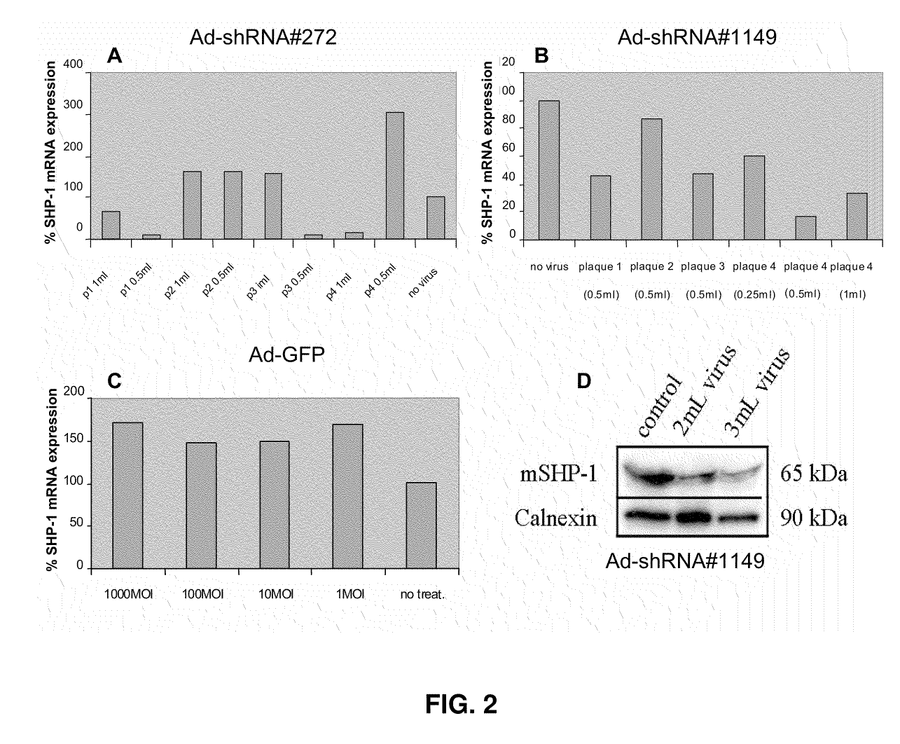

Two mouse SHP-1 specific small hairpin RNAs (shRNA) sequences, 272 and 1149, (referred to by their nucleotide position from the start site of the coding sequence in Genbank mRNA Accession #BC012660) were designed and cloned into the adenoviral vector pAd-BLOCK-iT-DEST RNAi (Invitrogen, Carlsbad, Calif.) which provides U6 polymerase II promoter-driven expression of the shRNA. The exemplary shRNA sequences Ad5-shRNA#1149 (SEQ ID NO:11) and Ad5-shRNA#272 (SEQ ID NO:12) are provided as follows: CACCGGAGCATGACACAGCAGAATACGAATATTCTGCTGTGTCATGCTCC (SEQ ID NO:11) and CACCGCACCATCATCCACCTTAAGTCGAAACTTAAGGTGGATGATGGTGC (SEQ ID NO:12), wherein sequence underlined in the shRNA is the exemplary SHP-1 specific palandromic 21-mer. Adenovirus carrying the appropriate sequence were produced and expanded in HEK-293 cells (ATCC Manassas, Va.). These viruses were then plaque purified and the ability to knock-down SHP-1 mRNA was tested in RAW cells, a murine macrophage-like cell line, by quantitative RT-PCR and are shown as a percent of the no virus treatment (FIG. 2A and FIG. 2B). Adenovirus expressing green fluorescent protein was used as a negative control for viral treatment (FIG. 2C). Ad-shRNA#1149 showed the greatest reduction in mRNA (>5 fold reduction) and was selected for subsequent studies. The ability of this shRNA to knock down SHP-1 protein was examined by western blot (FIG. 2D). A large scale, high titer, preparation of Ad-shRNA#1149 was produced by the Viral Vector Core Laboratory at Baylor College of Medicine and used in subsequent studies.

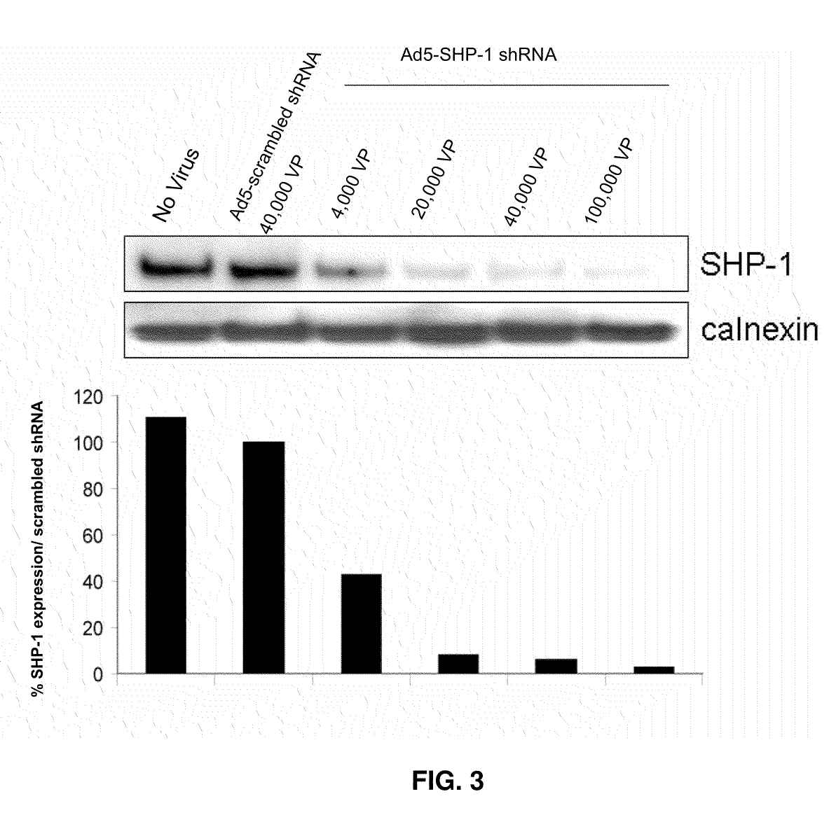

The high titer adenoviral preparation of SHP-1 specific Ad-shRNA#1149 (referred to from this point on as Ad5-SHP-1-shRNA), produced in the Viral Vector Core Laboratory at Baylor College of Medicine, was tested for its ability to knock-down endogenous SHP-1 protein in D2SC/1 cells, a murine dendritic-like cell line. As a control for adenoviral infection, a scrambled shRNA sequence was used (Ad5-scrambled-shRNA) that showed no significant sequence similarity to any known mouse gene as determined by a BLAST search of the NCBI Genbank nucleotide database. A titration of Ad5-SHP-1 shRNA viral particles was performed and SHP-1 protein expression was determined by western blot (FIG. 3). A 95% decrease in endogenous SHP-1 protein was observed using 40,000 viral particles/cell a viral dose which also resulted in minimal cell death. This dose was chosen for subsequent studies using mouse bone marrow derived dendritic cells (BMDCs).

FIG. 3 describes the knock-down of endogenous SHP-1 using high titer Ad5-SHP-1 shRNA. SHP-1 protein expression was measured in D2SC/1 cells following infection with Ad5-SHP-1 at varying doses as shown. Protein levels were analyzed by western blot with a SHP-1 specific antibody. As controls, SHP-1 expression is shown for uninfected cells and those infected with the Ad5-scrambled shRNA. The blot was also probed with a calnexin specific antibody as a loading control. Densitometry analysis of the western is shown below as a percentage of SHP-1 expression level relative to cells infected with Ad5-scrambled shRNA.

Phosphatase Dead Dominant Negative SHP-1 (dn-SHP-1)

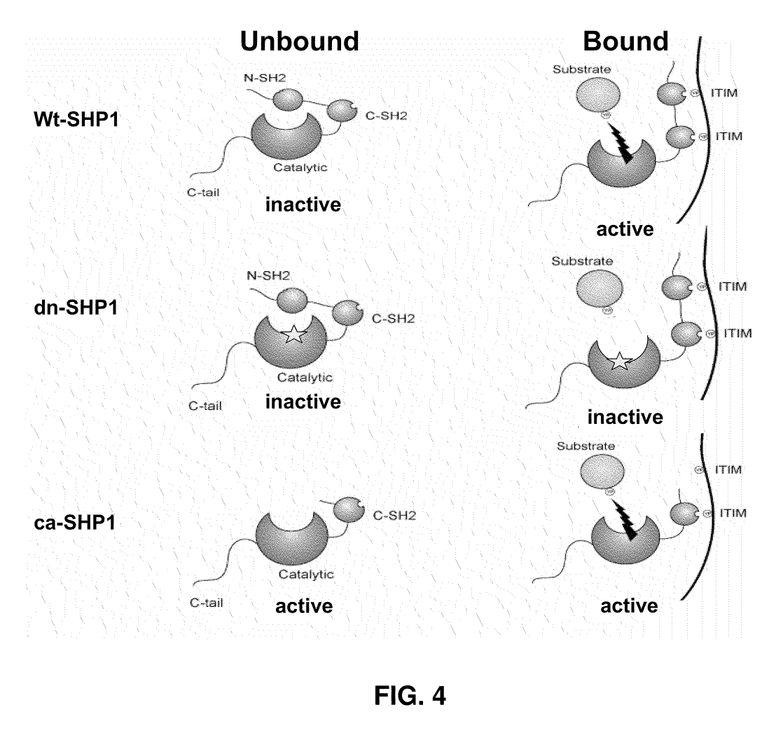

The wild type (wt) mouse SHP-1 sequence (wt-SHP-1; SEQ ID NO:7) by RT-PCR was generated from mouse spleen. Using a splice overlap extension strategy, the thymine at position 1503 (GenBank® Accession #BC012660; SEQ ID NO:10) was mutated to adenosine to create a cysteine to serine point mutant at position 453 (C453S) in the expressed protein that has previously been shown to abolish SHP-1 catalytic phosphatase activity. The C453S mutant has been shown to act as a dominant negative (dn-SHP-1; SEQ ID NO:8) by competitively binding to SHP-1 substrates and inhibiting endogenous SHP-1 phosphatase activity. In addition to creating the dn-SHP-1 construct to inhibit SHP-1 activity, a control construct in which SHP-1 activity was constitutive was created. Wild type SHP-1 is inactive in its native conformation due to the N-terminal SH2 domain blocking substrate access to the catalytic site (FIG. 4). Activation normally requires SH2 domain-dependent binding of SHP-1 to its cognate immunoreceptor tyrosine-based inhibitory motif (ITIM). Thus, a constitutively active (ca) SHP-1 mutant (SEQ ID NO:9) was generated by deleting the N-terminal SH2 domain of SHP-1 that is known to bind and sterically inhibit the catalytic site of SHP-1 when it is not bound to substrate. All three SHP-1 constructs (wt-, dn- and ca-SHP-1) were modified by the addition of a ten amino acid, N-terminal hemagglutinin (HA), coding sequence as an epitope tag to facilitate subsequent detection and differentiation from endogenous SHP-1. These constructs were cloned into the pAdTrack-CMV adenoviral expression vector.

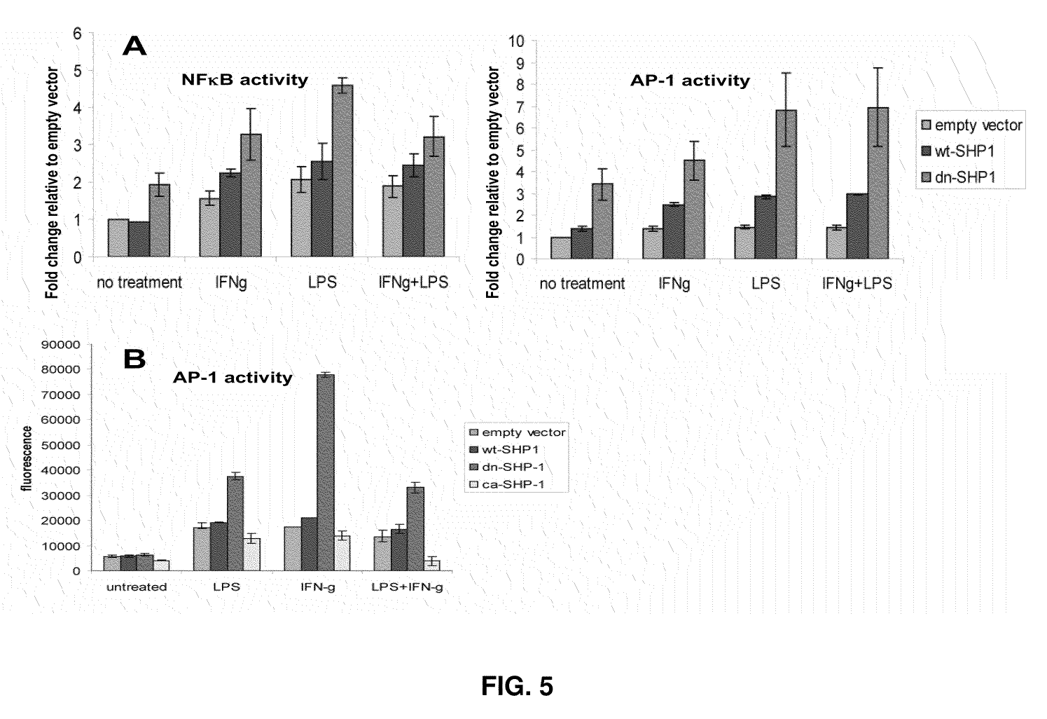

Function of the mutant and wt SHP-1 constructs was tested in RAW cells by transient transfection of SHP-1 vectors along with a reporter construct expressing a secreted alkaline phosphatase driven by either an NFκB or AP-1 dependent promoter. NFκB and AP-1 are major transcription factors stimulated by toll-like receptor (TLR) and cytokine signaling in immune system cells, and represent likely pathways of SHP-1 inhibition on cellular activation. Cells were transfected with the appropriate construct, reporter and then stimulated with interferon-γ (IFNγ) or bacterial lipopolysaccharide (LPS) as ligands for cytokine and TLR receptors respectively. In all studies transfection of the dn-SHP-1 construct enhanced both NFκB and AP-1 signaling in response to cytokine or TLR stimulation (FIG. 5A), demonstrating that the construct was functional and that SHP-1 normally inhibits these pathways. Transfection with the ca-SHP-1 construct showed the opposite effect to dn-SHP-1 by suppressing AP-1 signaling (FIG. 5B), again demonstrating that the construct was functional and that SHP-1 acts on this pathway.

### SHP-1 Modulates DC Migration Both In Vitro and In Vivo

Primary bone marrow-derived dendritic cells (BMDCs) were prepared from wild type mice in the following manner: Bone marrow cells were flushed from the femurs and tibias of C57BL/6 mice and cultured for 6 days in RPMI media supplemented with 10% FBS, 10 ng/mL IL-4 and 10 ng/mL GMCSF along with antibiotics. On day 6, dendritic cells (DCs) were either purified by magnetic bead assisted cell sorting (MACS; Miltenyi Biotec, Auburn, Calif.) prior to adenoviral transduction, or used as unpurified bulk DCs that were then transduced with adenovirus.

For DCs to initiate an immune response, they must capture antigen in the periphery and then migrate to the lymph nodes where they stimulate antigen specific T cells. To determine if SHP-1 signaling could affected the ability of DCs to migrate to draining lymph nodes, trafficking studies were performed both in vitro and in vivo. For the in vitro DC migration assays, unpurified bulk BMDCs were transduced with either Ad5-SHP1-shRNA or Ad5-scrambled-shRNA for 48 hours or left untreated. Half of the untreated cells were treated with 1 μg/mL LPS for 24 hours. Cells were collected and washed in serum-free media (SFM) and resuspended at 5×10⁶ cells/mL. 500 μL of SFM containing 100 ng/mL CCL21, a CCR7 ligand and one of the chemokines responsible for DC trafficking to lymph nodes in vivo, was used as the trafficking media and was added to the bottom chamber of a 24-well transwell plate (5 μm pores). Wells loaded with 500 μL of SFM without CCL21 were used as control for basal migration. 100 μL of the BMDC suspension was loaded in upper chamber of each transwell and was placed over the chambers containing the appropriate trafficking media. Transwell plates were incubated at 37° C. for 3 hours. BMDCs migrating into the lower chamber were counted using a hematocytometer.

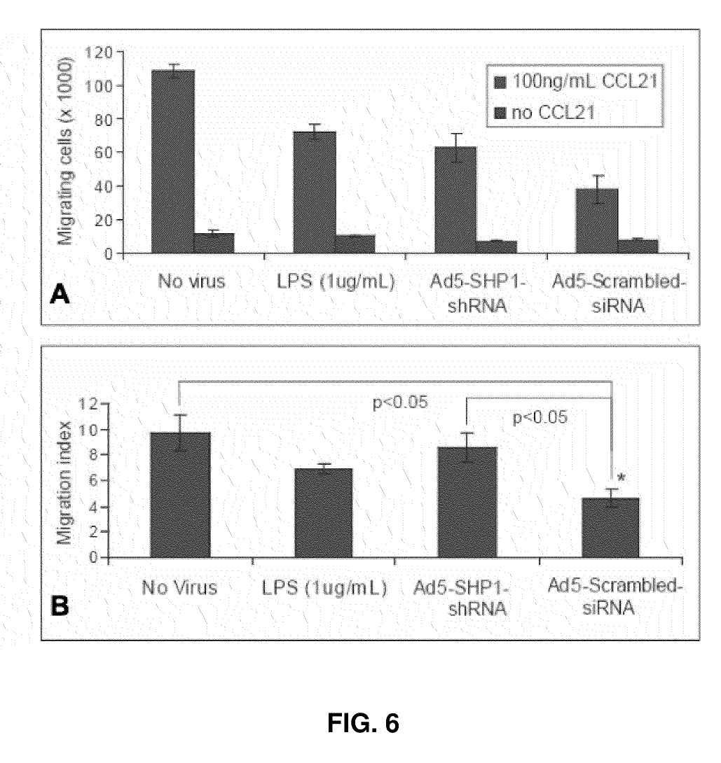

In all treatments exposure to CCL21 in the lower chamber markedly enhanced the rate of migration of DCs (FIG. 6A). Exposure of mouse BMDCs to LPS is known to cause a decrease in the rate of migration to CCR7 ligands, an effect that differs from that seen in human DCs. Similarly, exposure to adenovirus has also been shown to reduce the rate of migration in murine DCs. When BMDCs were transduced with Ad5-SHP-1-shRNA, their migration rate to CCL21 was not significantly different from that of untreated cells when compared to the background rate of migration in the absence of CCL21 (the migration index; FIG. 6B). This was in contrast to DCs transduced with the Ad5-scrambled-shRNA virus, which showed a significant reduction in migration index compared to both untreated cells and those treated with Ad5-SHP-1-shRNA (FIG. 6B).

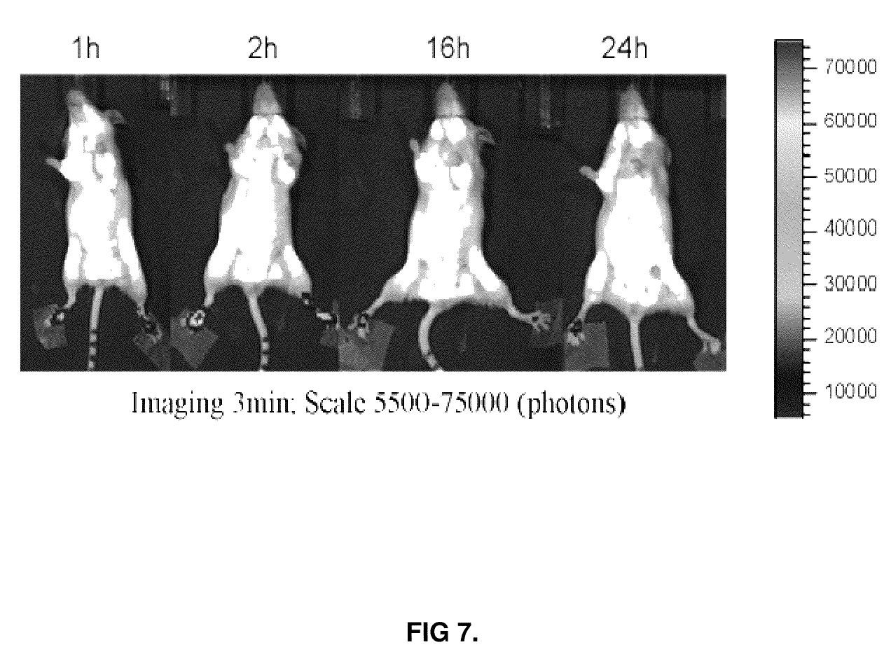

To determine if SHP-1 inhibition enhanced DC migration in vivo, BMDCs were prepared as described above but were transduced with an adenovirus expressing a clickbeetle red-shifted luciferase in addition to either Ad5-SHP-1-shRNA or Ad5-scrambled-shRNA. Transduced cells (2×10⁶) were injected into the contralateral footpads of tyrosinase-deficient albino C57BL/6 mice (B6(Cg)-Tyr^{c-2J}/J, Jackson Laboratory). At the specified intervals mice were injected i.p. with 100 μl of 10 mg/mL luciferin and imaged in vivo using IVIS® (Caliper Life Sciences, Hopkinton, Mass.). DCs treated with Ad5-SHP-1-shRNA could be seen to migrate out of the footpad by 2 hours post-injection and undetectable in the footpad by 24 hours post injection (FIG. 7 right side footpad). In contrast, DCs treated with the Ad5-scrambled-control-shRNA were still evident in the footpad at only marginally reduced levels even at 24 hours post-injection (FIG. 7 left side footpad). Taken together, these data indicate that SHP-1 modulates chemotaxis in mouse BMDCs and that inhibition of SHP-1 signaling enhances DC migration.

### SHP-1 Modulates DC Survival

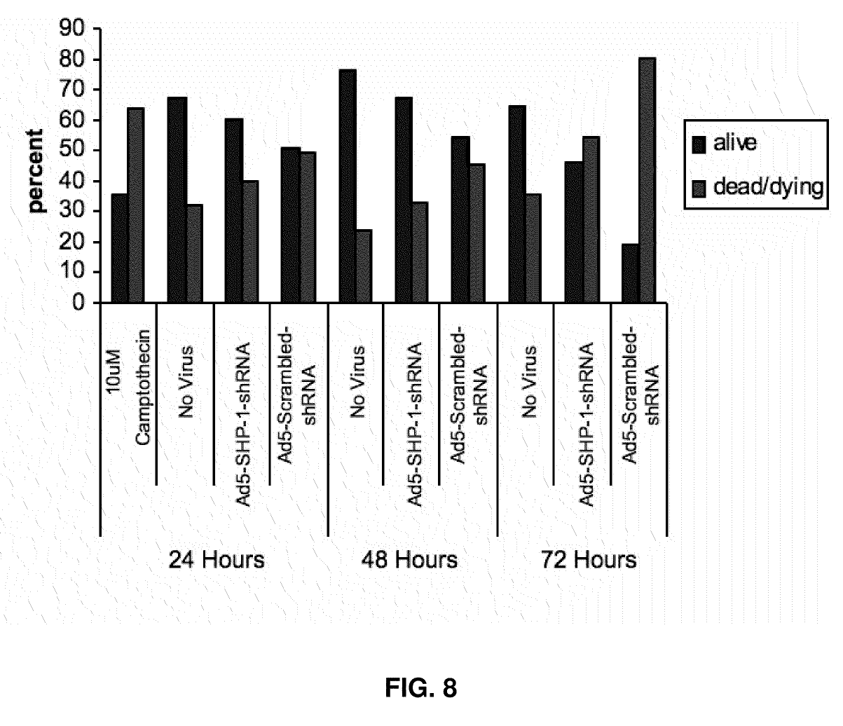

BMDCs were prepared as described above and purified by CD11c (a marker for DC) MACS. Purified DCs were transduced with either Ad5-SHP1-shRNA or Ad5-scrambled-shRNA or left untreated and the virus was washed away following a two hour exposure. Cell survival was determined at 24 hours, 48 hours and 72 hours after infection by annexin V and propidium iodide (PI) staining and analyzed by flow cytometry. Annexin V binds to phosphatidylserine and is an early marker of apoptosis. PI is a DNA intercalating dye that can only enter cells when their membrane is integrity is disrupted and is a marker of cells late in the apoptotic process. Viral infection of DCs causes cells to undergo apoptosis where approximately 50% were dead or dying within the first 24 hours and 85% were dead or dying by 72 hours post-infection (FIG. 8 Ad5-scrambled-shRNA). In contrast, cells treatment of with Ad5-SHP-1-shRNA showed greater viability with only 35% dead or dying within the first 24 hours and 55% dead or dying by 72 hours post-infection (FIG. 7 Ad5-SHP-1-shRNA). For cells not treated with virus 35% were dead or dying after 72 hours. This data indicates that SHP-1 signaling can promote apoptosis in DCs and that inhibiting SHP-1 leads to enhanced survival.

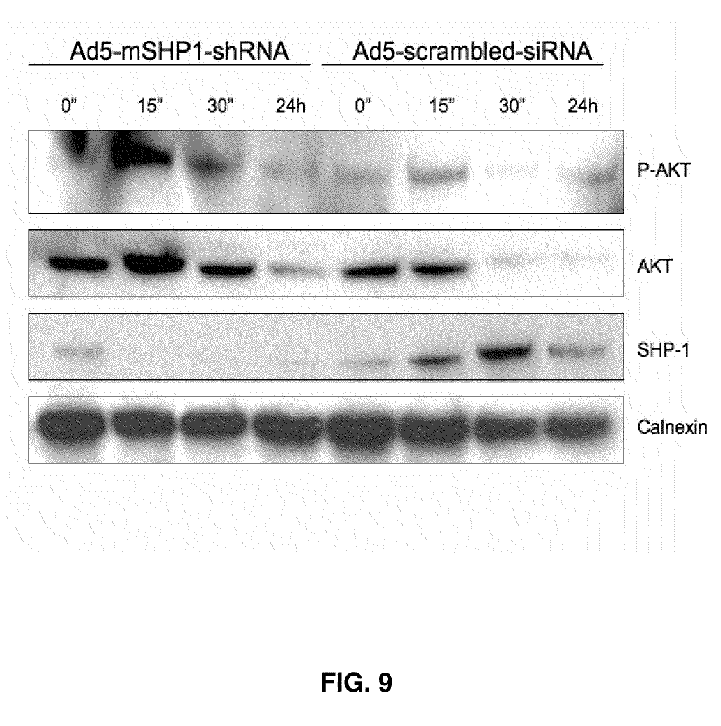

DC survival has been linked with activation of Akt/protein kinase B (PKB) family proteins, major effectors of phosphatidylinositol 3-kinase (PI3K) family members. LPS stimulated Akt signaling BMDCs was examined to determine if SHP-1 mediated survival might be working through this mechanism. MACS sorted for CD11c positive BMDCs were infected with either Ad5-SHP-1-shRNA or Ad5-scrambled-shRNA or left untreated. Cells (10⁷) per group were treated with 1 μg/mL LPS for the times indicated or left untreated. Lysates were analyzed by western blot for phosphorylated Akt. SHP-1 knock down enhanced LPS mediated Akt phosphorylation and also enhanced the steady state expression of total Akt protein (FIG. 9). This observation provides a mechanism for the increased survival seen when SHP-1 signaling was inhibited, in certain embodiments (FIG. 8). LPS stimulation also appears to enhance the total level of SHP-1 protein in DCs as shown by the SHP-1 blot for cells treated with Ad5-scrambled-shRNA. Taken together these data indicate that SHP-1 signaling can promote apoptosis in DCs through the inhibition of Akt phosphorylation and that inhibiting SHP-1 leads to enhanced DC survival.

SHP-1 Knock Down Enhances CD8⁺ Effectors and CD4⁺ Th1 while Inhibiting FOXP3⁺ Treg Induction In Vivo

To determine the effect of SHP-1 inhibition on the initiation of T cell responses, BMDCs were cultured and CD11c⁺ MACS purified as described above. DCs were loaded with one of 3 different peptide tumor antigens: 1) tyrosinase-related protein 2 (Trp-2; SVYDFFVWL; SEQ ID NO:13) that binds to H-2K^{b} and is specific for the B16 murine melanoma tumor line; 2) six transmembrane antigen of the prostate (STEAP₃₂₇₋₃₃₅; VSKINRTEM; SEQ ID NO:4) that binds to H-2D^{b} and is specific for transgenic adenocarcinoma of the mouse prostate (TRAMP) tumors; or 3) prostate stem cell antigen (PSCA₂₉₋₃₇; AQMNNRDCL; SEQ ID NO:6) that binds to H-2Db and is specific for TRAMP tumors. Following peptide loading DCs were left untreated or transduced with one of 3 different adenoviral vectors: 1) Ad5-SHP-1-shRNA; 2) Ad5-scrambled-shRNA; or 3) Ad5-CMV-empty, an adenovirus carrying a CMV promoter expression vector but no insert. Ad5-CMV-empty was used as an additional negative control to the Ad5-scrambled-shRNA virus, to demonstrate that Ad5-scrambled-shRNA did not have any specific RNAi activity that might facilitate DC inhibition. Using all combinations of peptide and virus treatment yielded 9 experimental vaccines and one no vaccine control. Vaccines (2×10⁶ DCs) were injected i.p. into wt C57BL/6 mice. Seven days following vaccination mice were sacrificed and total splenocytes were analyzed by multi-color flow cytometry for the expression of several T cell subsets. CD3⁺ CD8⁺IFNγ⁺ cells were characterized as CTL effectors, CD3⁺ CD4⁺IFNγ⁺ cells were characterized as Th1 helper T cells, and CD4⁺ FOXP3⁺ cells were characterized as Treg cells. Flow cytometry data were analyzed by one-way analysis of variance (ANOVA) and Tukey-Kramer HSD multiple comparisons test for percentage of cells falling within each population following treatment. Cells were stained with anti-CD3, anti-CD8 and anti IFNγ to differentiate CD8⁺ effectors and CD4⁺ Th1 T cell skewing or anti-CD4 and anti-FOXP3 to determine Tregs. To determine if there was a peptide specific effects between the 3 different tumor antigens, viral treatments were pooled for each peptide exposure. Data represent the averages 6 mice per peptide treatment group and 2 mice in the no treatment control group. No significant differences were seen between peptides in the induction of Tregs or CD8⁺ effector cells. Th1 skewing was significant between STEAP and Trp-2 peptides and the no treatment control (ANOVA: df=3, F=3.67, p<0.05 Tukey-Kramer HSD q*=2.91, p<0.05) indicating that DC vaccination with any peptide combination induces a Th1 response.

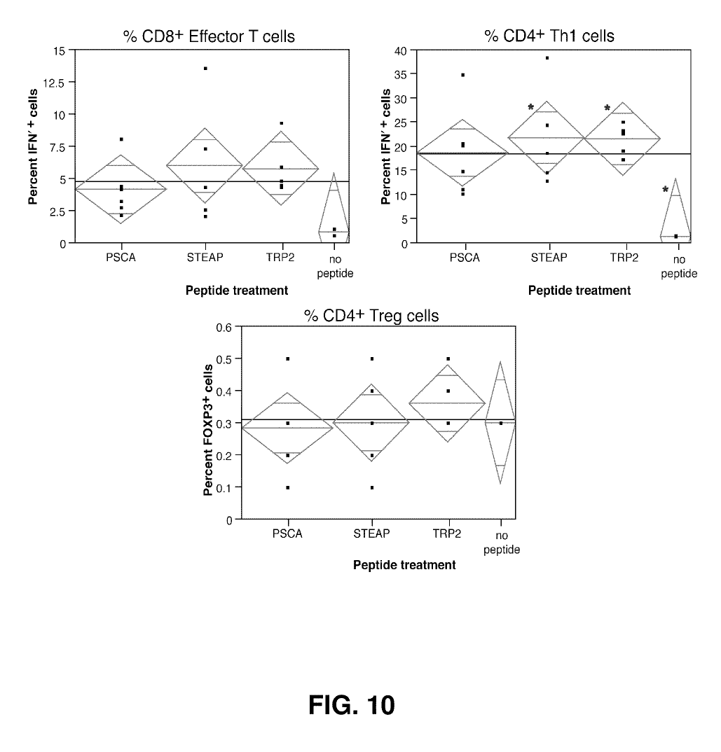

FIG. 10 shows that DC vaccination enhances Th1 skewing of T cells. BMDCs vaccines were loaded with one of 3 different peptide tumor antigens: 1) Trp-2 (SVYDFFVWL; SEQ ID NO:13); 2) STEAP₃₂₇₋₃₃₅ (VSKINRTEM (SEQ ID NO:4); or 3) PSCA₂₉₋₃₇ (AQMNNRDCL; SEQ ID NO:6)). Following peptide loading DCs were left untreated or transduced with one of 3 different adenoviral vectors: 1) Ad5-SHP-1-shRNA; 2) Ad5-scrambled-shRNA; or 3) Ad5-CMV-empty, an adenovirus carrying a CMV promoter expression vector but no insert. Vaccines were injected i.p. into wt C57BL/6 mice and T cell skewing analyzed 7 days later by flow cytometry from total splenocytes. Cells were stained with anti-CD3, anti-CD8 and anti IFNγ for effector and Th1 cells or anti-CD4 and anti-FOXP3 for Tregs. For the peptide specific analysis viral treatments were pooled for each peptide exposure. Data represent the averages 6 mice per peptide treatment and two mice in the no treatment control group. The green diamonds represent the mean and 95% confidence interval for each group from the ANOVA. The black line is the mean of means for the study and red asterisk (*) indicates significant differences (p<0.05 by Tukey-Kramer HSD multiple comparisons test).

To determine if there was a SHP-1 specific effect between the viral treatments, peptide treatments were pooled for each viral exposure. Data represent the averages 6 mice per viral treatment group and 2 mice in the no treatment control group. SHP-1 specific knock down with Ad5-SHP-1-shRNA induced a significantly higher proportion of CD8⁺ effector cells control (ANOVA: df=3, F=5.06, p<0.02 Tukey-Kramer HSD q*=2.91, p<0.05) and CD4⁺ Th1 skewed T cells control (ANOVA: df=3, F=9.01, p<0.002 Tukey-Kramer HSD q*=2.91, p<0.05) compared to the untreated (no vaccine) control. Treatment with the Ad5-scrambled-shRNA and the Ad5-CMV-empty controls showed a trend towards increased CD8⁺ effector cells and CD4⁺ Th1 T cells but these increases were not significantly different from either the no treatment control or the Ad5-SHP-1-shRNA treated vaccines. Examination of the effect of SHP-1 knock down on the induction of Tregs showed that inhibition of SHP-1 significantly decreased the percentage of Tregs compared to the empty viral control (ANOVA: df=3, F=3.50, p<0.05 Tukey-Kramer HSD q*=2.91, p<0.05). No significant differences were seen between the control virus treated groups or between the control treated and the untreated controls. Taken together these data indicate that inhibiting SHP-1 in DC vaccines significantly increases the induction of CTL responses and Th1 skewing indicating the likelihood of an enhanced anti-tumor immune response. Supporting this, is the fact that in addition to effector CD8⁺ CTL increases, SHP-1 correspondingly diminishes the suppressive Treg response indicating an even greater anti-tumor effect may be achieved.

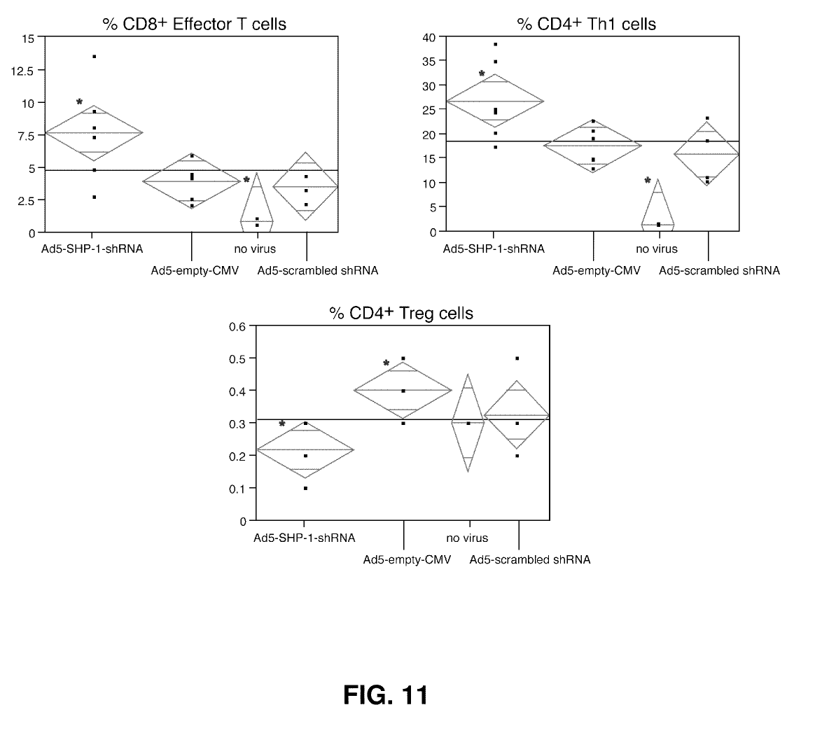

FIG. 11 shows that SHP-1 knock down enhances CD8⁺ effectors and CD4⁺ Th1 while inhibiting FOXP3+Treg induction. BMDCs vaccines were loaded with one of 3 different peptide tumor antigens: 1) Trp-2 (SVYDFFVWL; SEQ ID NO:13); 2) STEAP₃₂₇₋₃₃₅ (VSKINRTEM (SEQ ID NO:4); or 3) PSCA₂₉₋₃₇ (AQMNNRDCL; SEQ ID NO:6). Following peptide loading DCs were left untreated or transduced with one of 3 different adenoviral vectors: 1) Ad5-SHP-1-shRNA; 2) Ad5-scrambled-shRNA; or 3) Ad5-CMV-empty, an adenovirus carrying a CMV promoter expression vector but no insert. Vaccines were injected i.p. into wt C57BL/6 mice and T cell skewing analyzed 7 days later by flow cytometry from total splenocytes. Cells were stained with anti-CD3, anti-CD8 and anti IFNγ for effector and Th1 cells or anti-CD4 and anti-FOXP3 for Tregs. For the SHP-1 specific analysis viral treatments were pooled for each viral treatment. Data represent the averages 6 mice per peptide treatment and 2 mice in the no treatment control group. The green diamonds represent the mean and 95% confidence interval for each group from the ANOVA. The black line is the mean of means for the study and red * indicate significant differences (p<0.05 by Tukey-Kramer HSD multiple comparisons test).

### Creating Tumor Cell Lines Stably Expressing Red-Shifted Luciferase for In Vivo Imaging of Ectopic and Metastatic Tumors in Live Animals

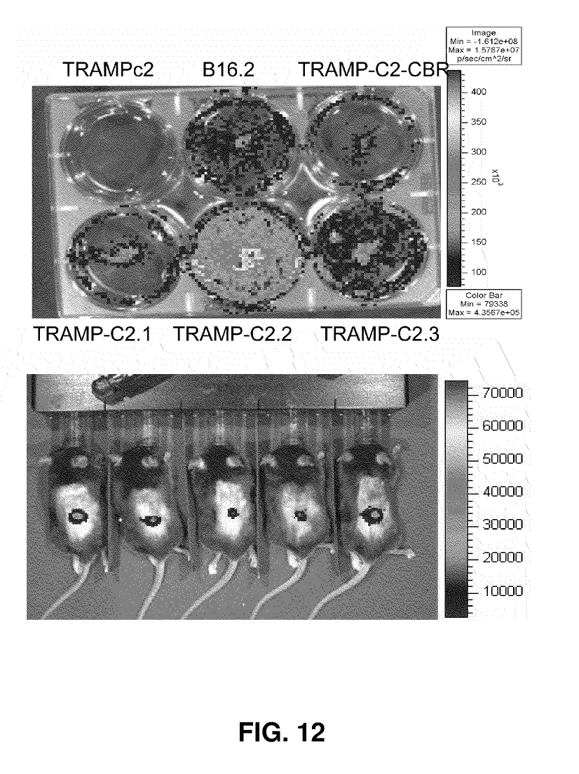

Tumor lines that stably express a red-shifted luciferase were created in order to monitor the size and location of model tumors in living animals using IVIS™ optical bioluminescence imaging. These various tumor lines were transfected with a luciferase expression vector, cloned by limiting dilution and selected for the brightest expression when exposed to the substrate luciferin (FIG. 12). As a proof of principle for using these tumor lines, the luciferase-transfected B16 and TRAMP C-2 tumor lines were tested for growth in wt C57BL/6 mice. These results showed that luciferase-transfected tumors grew substantially slower that the untransfected tumors in vivo. In addition, the transfected tumor lines were for the most part resolved by the animals in the absence of any vaccination.

FIG. 12 Luciferase expressing glow tumors. Clones of luciferase expressing B16 and TRAMP C-2 tumor lines growing in vitro (upper panel). TRAMP C-2 glow tumors were injected s.c. into C57BL/6 mice and are shown 3 days post-injection of 7×10⁶ cells using IVIS® imaging (Caliper Life Sciences, Hopkinton, Mass.).

### SHP-1 Knock Down Enhances DC Vaccine Efficacy Against B16 Melanoma and TRAMP C-2 Prostate Tumors

Since it was found that SHP-1 inhibition enhanced DC activation signaling, migration, survival, and CD8⁺ effector function, the next step was to determine if inhibiting SHP-1 in DCs would enhance their function as anti-tumor vaccines. To test this in a prostate cancer model in vivo TRAMP C2 cells were injected subcutaneously on the dorsal flank of C57BL/6 mice. Unfortunately, there have been no good tumor antigens previously defined for the TRAMP model. Recent studies have shown, however, that the six transmembrane epithelial antigen of the prostate (STEAP) is a good candidate for immunotherapies in human prostate cancer. In addition, a recent study showed that the mouse homolog of STEAP (which is 80% identical to the human protein) and the mouse homolog of prostate stem cell antigen (PSCA) were expressed at high levels in TRAMP C2 cells.

Two online epitope prediction algorithms Bimas (see the world website at NIH website) and SYFPEITHI (see the website of the same name) were used to scan their amino acid sequences and to determine peptide epitopes from these proteins that were potentially immunoreactive. Predictions of peptides binding to the MHC class 1 molecules H-2K^{b} and H-2D^{b} from the C57BL/6 background, yielded a number of candidate epitopes. Of these candidates, epitopes chosen were either the strongest predicted binders or had sequences similar to published human epitopes which indicated that they were likely to be processed in vivo. The five epitopes chosen for testing are shown in Table 1 and include OVA peptide, as a comparison for strong H-2K^{b} binding.

TABLE 1

| | | | |
|---|---|---|---|
| | AA | | |
| Peptide Epitope | Sequence | Bimas* | SYFPEITHI |

| | | | | |
|---|---|---|---|---|
| OVA258-265 | SIINFEKL | 17.4 | (Kb) | 25 (Kb) |
| STEAP186-192 | RSYRYKLL | 132 | (Kb) | 29 (Kb) |
| STEAP84-91 | LTFLYTLL | 48 | (Kb) | 22 (Kb) |
| STEAP327-335 | VSKINRTEM | 718.829 | (Db) | 26 (Db) |
| STEAP262-270 | LLLGTVHAL | 4.311 | (Db) | 12 (Db) |
| PSCA29-37 | AQMNNRDCL | 10838.473 | (Db) | 25 (Db) |

In Table 1, there are SIINFEKL (SEQ ID NO: 1); RSYRYKLL (SEQ ID NO: 2) LTFLYTLL (SEQ ID NO: 3); VSKINRTEM (SEQ ID NO: 4); LLLGTVHAL (SEQ ID NO: 5); and AQMNNRDCL (SEQ ID NO: 6).

In Table 1, OVA Kb binding peptide SIINFEKL was used as control for a known good binder. Four peptides were chosen from the STEAP-1 protein that showed a high ranking for predicted binding affinity or showed sequence homology to the known human HLA-A*0201 binding epitopes from human STEAP-1 (indicating the peptide is likely to be processed in vivo and one PSCA peptide was chosen because of its strong predicted binding affinity).

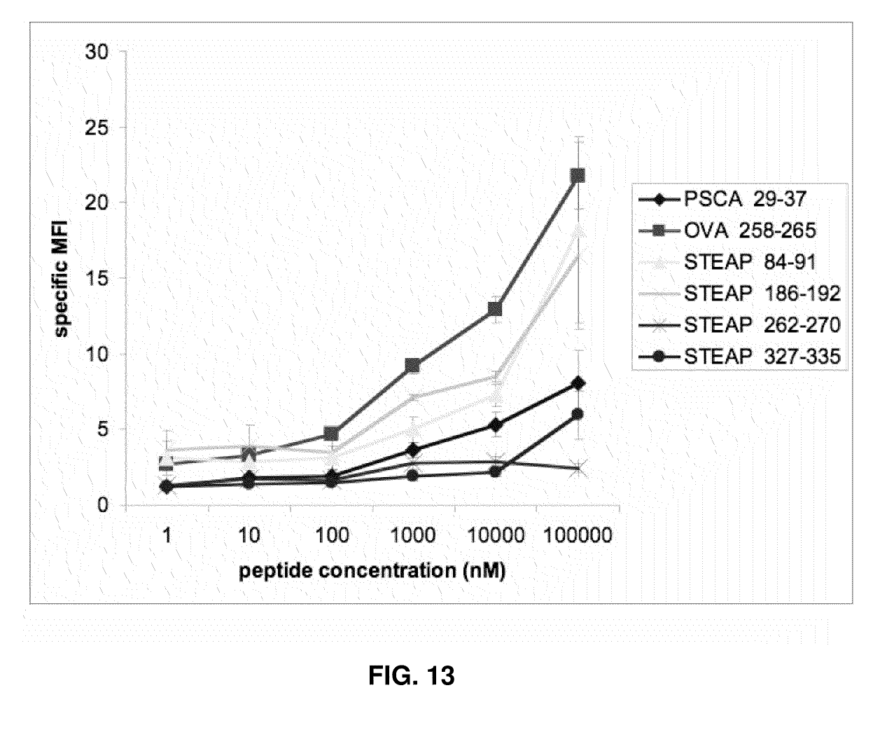

FIG. 13 shows relative binding affinities of STEAP and PSCA peptides predicted to bind H-2K^{b} or H-2D^{b}. 10⁶ RMA-S cells were incubated overnight at 37° C. with each of the five predicted at the concentrations indicated. Cells were stained with antibodies for H-2K^{b} (Y-3; ATCC-HB176) or H-2D^{b} (28-14-8S; ATCC-HB27) followed by a goat anti-mouse-FITC second step and MHC class I surface expression was analyzed by flow cytometry. Specific MFI is the mean fluorescent intensity of the sample-mean fluorescent intensity of goat anti-mouse-FITC second step alone. Error bars represent the standard deviation of triplicate measurements. This is representative of 3 separate studies.

The binding affinity of the five predicted peptides was tested. In a surface stabilization assay using the TAP1 deficient cell line RMA-S. RMA-S cells were pulsed with peptide, at the indicated concentrations, and incubated overnight. Cells were stained with antibodies for H-2K^{b} (Y-3; ATCC-HB176) or H-2D^{b} (28-14-8S; ATCC-HB27) followed by a fluorescent-labeled secondary antibody and MHC class I surface expression was analyzed by flow cytometry. All peptides bound to their expected class 1 molecules with the exception of STEAP₂₆₂₋₂₇₀ that showed no detectable binding at any peptide concentration. STEAP₈₄₋₉₁ and STEAP₁₈₆₋₁₉₂ bound with an affinity near that of OVA₂₅₈₋₂₆₅, a well characterized strong binding H-2K^{b} epitope from chicken ovalbumin. Although PSCA₂₉₋₃₇ and STEAP₃₂₇₋₃₃₅ did not appear to bind as well as some of the other peptides they are predicted to bind to H-2D^{b} not H-2K^{b}. Because there was no positive control strong binding peptide for H-2D^{b} in these studies, there is a possibility that the “lower” binding affinity of these peptides may be due to a lower relative expression of H-2D^{b} on RMA-S cells compared to H-2K^{b}, or differences in the binding affinities of the different antibodies use to detect each molecule. Taken together, this data indicate that at least 4 of the 5 predicted peptide epitopes for TRAMP C2 tumors could act as immunoreactive antigens when administered in vivo as part of an anti-tumor vaccine.

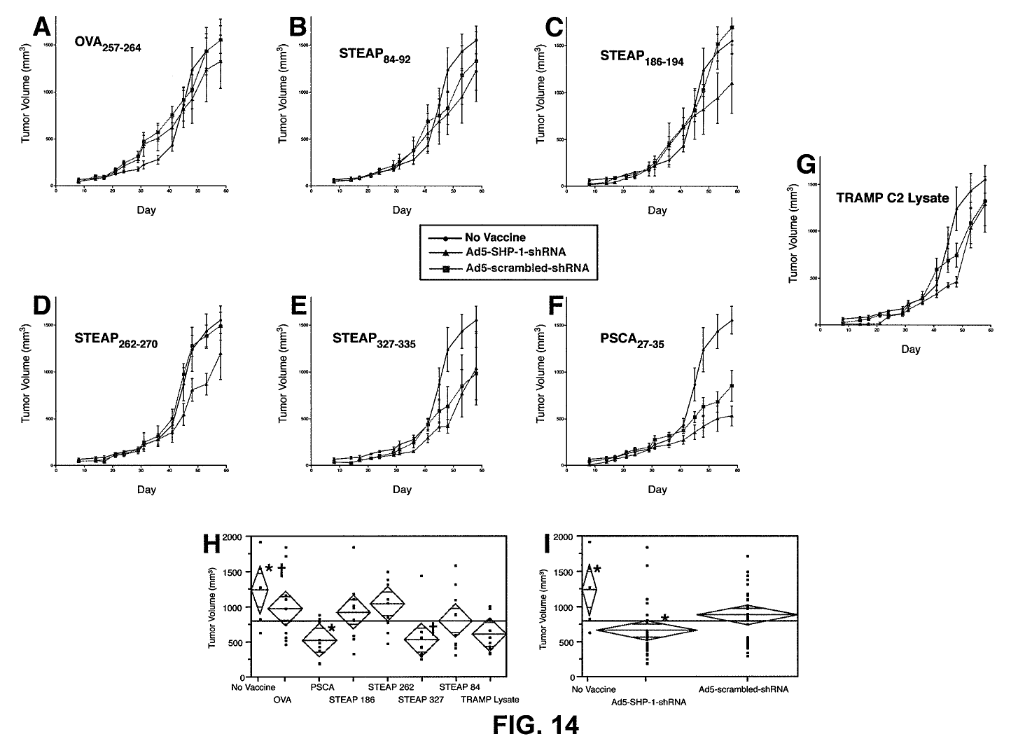

FIG. 14 shows that SHP-1 inhibition enhances DC vaccines against TRAMP C2 tumors. BMDCs were prepared as described above, transduced with either Ad5-SHP-1-shRNA or the control Ad5-scrambled-shRNA at 40,000 viral particles/cell, and pulsed with one of the six peptides listed in Table 1 (OVA, an irrelevant epitope to TRAMP 2 tumors, served as a negative control peptide) or a lysate of TRAMP C2 cells as a positive control antigen. 6-8 week C57BL/6 mice, bearing TRAMP C2 tumors (7×10⁶ cells injected s.c. on the dorsal flank three days prior to vaccination), were given a single i.p. vaccination with 2×10⁶ treated DCs or left untreated. Tumors were measured (length and width using calipers) every 2-4 days until termination of the study and tumor volume was estimated using the formula: Tumor volume (mm3)=x²y×0.5236, where x is the smaller tumor dimension, and y is the larger tumor dimension. A-G) TRAMP tumor growth curves for vaccines loaded with the indicated peptides. Error bars represent the standard error of the mean of 5 mice vaccinated in each treatment. These data are from one of 2 separate studies each showing similar results. H-I) ANOVA analysis of peptide function and SHP-1 function. Data represent the averages 10 mice per peptide treatment and 5 mice in the no treatment control group for H) and 35 mice per viral transduction treatment and 5 mice in the no treatment control group for I). The green diamonds represent the mean and 95% confidence interval for each group from the ANOVA. The black line is the mean of means for the study and red symbols indicate significant differences (p<0.05 by Tukey-Kramer HSD multiple comparisons test).

In mice not receiving any vaccination ectopic TRAMP C2 tumors grow exponentially and reach maximum allowable size (10% of body weight, 2000-3000 mm³ depending on the age of the mice) in 75-85 days (determined empirically from pilot experiments, data not shown). The growth rate of ectopic TRAMP C2 tumors in mice vaccinated with DCs loaded with the irrelevant control peptide, OVA, was equivalent to mice receiving no vaccine treatment (FIG. 14A). These data indicate that DCs alone in the absence of a cognate tumor antigen cannot stimulate an anti-tumor response. Mice vaccinated with DCs loaded with one of the STEAP or PSCA peptides or the TRAMP C2 lysate, all showed a similar trend of decreased tumor growth rate compared to the untreated mice (FIG. 14B-G). On closer inspection of the differences between the mean tumor volumes (5 mice/treatment), two peptides generated strong inhibition of tumor growth, PSCA₂₉₋₃₇ and STEAP₃₂₇₋₃₃₅ (FIGS. 14E and F). To determine if there was a significant difference between the various peptides used in these DC vaccinations, mice were pooled across viral treatments (Ad5-SHP-1 shRNA or Ad5-scrambled-shRNA) employing the same peptide. An ANOVA for differences in mean tumor volume at day 48 post-vaccination (the time at which the majority of the vaccinated tumor growth curves change slope) and Tukey-Kramer HSD multiple comparisons test. A significant difference was seen between peptide treatments (ANOVA: df=7, F=3.80, p<0.005). Multiple comparisons testing showed that there was a significant decrease in tumor growth rate for mice treated with either PSCA₂₉₋₃₇ or STEAP₃₂₇₋₃₃₅ loaded DC vaccines compared to mice receiving no vaccination Tukey-Kramer HSD q*=3.13, p<0.05; FIG. 14H). No significant differences were shown between untreated mice and other peptide vaccinations.

Next experiments were conducted to determine if vaccines transduced with Ad5-SHP-1-shRNA inhibited tumor growth compared to transduction with Ad5-scrambled-shRNA or no vaccine treatment, irrespective of what peptide was used in the vaccine. To determine if SHP-1 knock down had an effect on vaccine efficacy, mice were pooled across all peptide treatments and an ANOVA and Tukey-Kramer HSD multiple comparisons test were performed (FIG. 14I). Vaccines where SHP-1 was deficient showed significantly lower tumor volumes compared with those that were untreated. There were no significant differences in tumor volume between untreated mice and mice treated with Ad5-scrambled-shRNA vaccines (ANOVA: df=2, F=5.69, p<0.01; Tukey-Kramer HSD q*=2.93, p<0.05). This experiment was repeated and identical results were obtained for both SHP-1 and peptide effects in both experiments. Taken together these data demonstrate that SHP-1 inhibition significantly enhances DC vaccine efficacy against TRAMP prostate tumors in vivo. In addition, these data show that two new MHC class I tumor epitopes expressed in TRAMP C2 tumors in vivo have been defined and can be utilized for anti-tumor immunotherapy in this animal model.

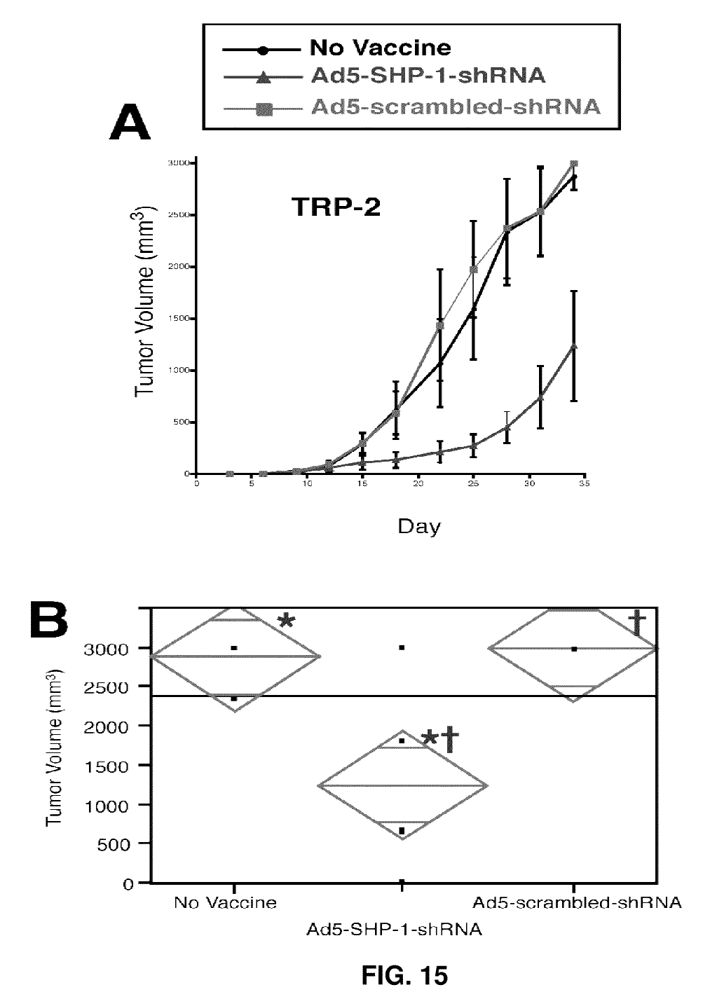

In an effort to further demonstrate that SHP-1 inhibition is effective in enhancing anti-cancer responses, against tumors other than the TRAMP C2 model, vaccine experiments were performed on mice bearing ectopic B16 melanoma tumors. The B16 melanoma, which is both aggressive and poorly immunogenic, expresses tyrosinase related protein-2 (Trp-2) of which peptide, SVYDFFVWL (SEQ ID NO:13), is recognized by CD8⁺ T cells in the context of H-2K^{b}. B16 represents a much more difficult tumor to treat than TRAMP and is considered the “gold standard” for ectopic tumor models in the C57BL/6 background. BMDCs were prepared as described above, pulsed with the Trp-2 and then transduced with either Ad5-SHP-1 shRNA or the control Ad5-scrambled-shRNA virus at 40,000 viral particles/cell. 6-8 week C57BL/6 mice, bearing B16 tumors (5×10⁵ cells injected s.c. on the dorsal flank three days prior to vaccination), were given a single i.p. vaccination with 2×10⁶ treated DCs or left untreated. Tumors were measured every 2-4 days until termination of the experiment and tumor volume was calculated as described above. Even with an inoculation 14 fold lower than that used for TRAMP C2 tumors, B16 tumors grow significantly faster and reach maximum acceptable size in 25-35 days in untreated mice (determined empirically from pilot experiments, data not shown). Mice vaccinated with DCs transduced with Ad5-SHP-1-shRNA showed markedly smaller mean tumor volumes (5 mice/treatment) beginning at day 18 than those vaccinated with Ad5-scrambled-shRNA transduced DCs or untreated mice (FIG. 15A). To determine if this difference was statistically significant, ANOVA and Tukey-Kramer HSD multiple comparisons test were performed. Mice vaccinated with the SHP-1 deficient vaccine showed significantly lower mean tumor volume compared with either control group (ANOVA: df=2, F=9.82, p<0.005; Tukey-Kramer HSD q*=2.67, p<0.05; FIG. 15B). This experiment was repeated and the same SHP-1 effect was seen in both experiments. These data demonstrate that SHP-1 inhibition significantly enhances DC vaccine efficacy against B16 tumors in vivo.

FIG. 15 shows that SHP-1 inhibition enhances DC vaccines against B16 tumors. BMDCs were prepared as described above, pulsed with the Trp-2 and then transduced with either Ad5-SHP-1 shRNA or the control Ad5-scrambled-shRNA virus at 40,000 viral particles/cell. 6-8 week C57BL/6 mice, bearing B16 tumors (5×10⁵ cells injected s.c. on the dorsal flank three days prior to vaccination), were given a single i.p. vaccination with 2×10⁶ treated DCs or left untreated. Tumors were measured every 2-4 days until termination of the experiment and tumor volume was calculated as described above. A) B16 tumor growth curves for vaccines loaded with Trp-2 peptide and transduced with viral vectors as indicated. Error bars represent the standard error of the mean of 5 mice vaccinated in each treatment. These data are from one of 2 separate experiments each showing similar results. B) ANOVA analysis SHP-1 function. The green diamonds represent the mean and 95% confidence interval for each group from the ANOVA. The black line is the mean of means for the experiment and red symbols indicate significant differences (p<0.05 by Tukey-Kramer HSD multiple comparisons test.

The results of the B16 and TRAMP tumor experiments (4 independent experiments in 2 different tumor models) clearly demonstrate that SHP-1 signaling in DCs constitutes a major inhibitory pathway, significant in its ability to down-regulate the initiation of antigen specific CD8⁺ T cell responses in vivo. These tumor data are augmented by the mechanistic data indicating that SHP-1 inhibition, enhances DC activation signaling, survival, migration, and the ability of DCs to skew signaling towards a pro-inflammatory CD4⁺ Th1 immune response while inhibiting Treg induction. The implication of these data in concert, is that SHP-1 signaling is a feasible protein to target in the design and implementation of DC based vaccines against tumors and potentially against other infectious diseases.

### Example 2

### Exemplary Methods and Reagents for Blocking SHP-1 Function

Exemplary embodiments of methods and compositions demonstrating that SHP-1 inhibition is effective in enhancing anti-cancer responses are provided herein.

SHP-1 is a significant inhibitor of a number of key signaling pathways crucial for DC activation, migration and antigen processing. Blocking SHP-1 function in DC used as cell-based cancer vaccines enhances their therapeutic efficacy, increasing anti-tumor specific CTL leading to a reduction in tumor burden. The efficacy of SHP-1 inhibited DC vaccines in several murine tumor models including both ectopic and orthotropic prostate cancer is tested. In addition to testing SHP-1 inhibition alone, also test its effect in combination with DC stimulation through an inducible CD40 construct (iCD40) which has been shown to have efficacy against some tumor models.

Two strategies for inhibiting SHP-1 activity in DC are engineered, small interfering RNA knockdown and over-expression of a phosphatase dead dominant negative mutant.

SHP-1 Specific shRNA

Two human and two murine anti-SHP-1 shRNA constructs have been sequenced and cloned into the adenoviral gateway vector (Invitrogen). Additional tests of these vectors include, their ability to knockdown native SHP-1 expression in the human Jurkat TAg cell line and in the mouse D2SC/1 dendritic cell line.

Phosphatase Dead Dominant Negative SHP-1 (dnSHP-1)

The human and mouse SHP-1 was cloned and sequenced by RT-PCR. Phosphatase activity of SHP-1 is completely abrogated by mutating the cysteine at position 453 to a serine (Gupta et al., 1997). Using a splice overlap extension strategy, the native sequence is mutated to derive dnSHP-1 which is cloned into an epitope tagged adenoviral vector. Test for this construct include its ability to interfere with native SHP-1 activity in the mouse dendritic cell line D2SC/1 and in bone marrow derived primary DC.

### In Vivo Imaging of Tumors in Mice

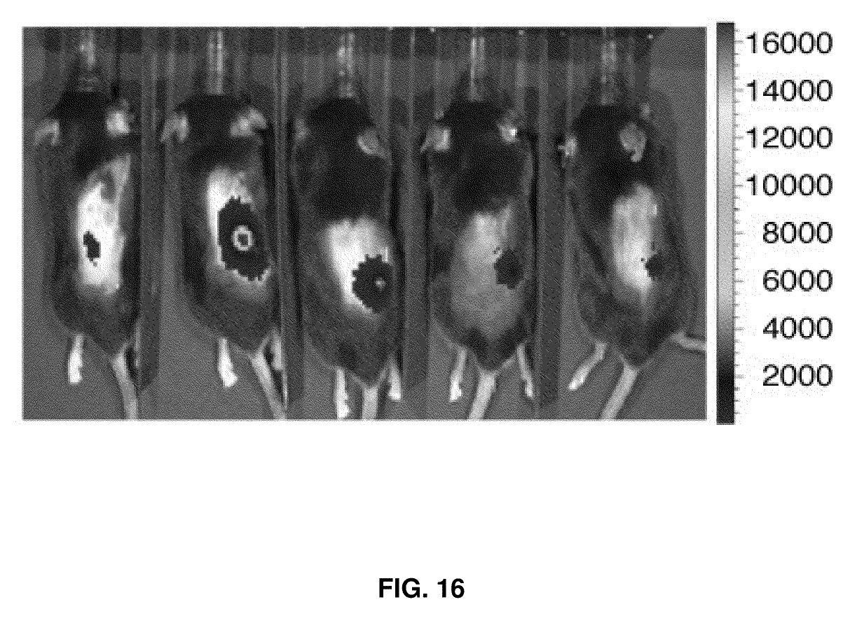

The tumor cell lines EG.7-OVA (Suzue et al., 1997) and B16 (Overwijk et al. 1999) were transfected with a vector containing a red shifted click-beetle luciferase (rs-Luc) (Viviani et al., 2002). Tumor lines expressing the marker were selected by antibiotic resistance and cloned by limiting dilution. After initial testing in vitro to select for high luciferase expression, clones were tested in vivo as follows: C57BL/6 mice were injected s.c. with 105 tumor cells. After 5 days when most mice had developed at least a small palpable tumor, mice were injected i.p. with d-luciferin, anesthetized with isofluorane and the tumors were imaged and quantified (FIG. 16) using the CCD-based IVIS™ Imaging System (Xenogen).

FIG. 16 shows the in vivo imaging of subcutaneous B16 tumors in mice. Mice were inoculated s.c. with 10⁵ B16 tumor cells expressing rs-Luc. After 5 days, mice were anaesthetized and injected with 100 ml d-luciferin (15 mg/ml) i.p., 15′ later mice were imaged for 30″ with an IVIS™ Imaging system.

### Production of Purified Bone Marrow Derived Dendritic Cells:

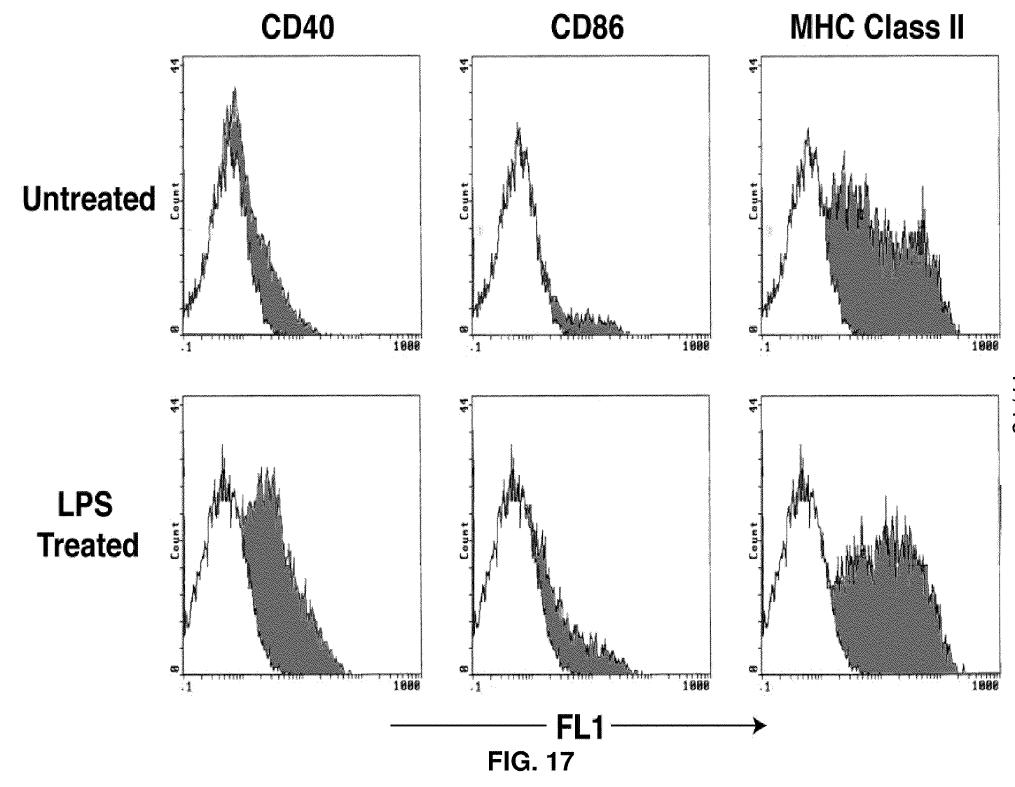

C57BL/6 bone marrow cells were purified on a Lympholyte™ (Cedarlane) gradient and cultured in media containing GM-CSF and IL-4 for 7 days. Cells were isolated from the culture using MACS anti-CD11c beads (Miltenyi Biotech, Germany) yielding ≧94% CD11c⁺ DC. To test for DC function, half of the cells were treated with LPS for 48 hours. The cells were analyzed by flow cytometry for DC maturation markers CD40, CD86 and I-A^{b} (MHC class II). LPS treatment of DC did not further increase class II surface expression, which was already high on the untreated cells, but did upregulate CD40 and CD86 to show a mature phenotype (FIG. 17).

FIG. 17 shows that bone marrow derived DC are matured by LPS. Bone marrow lymphocytes were cultured for 7 days in GM-CSF and IL-4 before further purification on an anti-CD11c column. CD11c⁺ DC were incubated in LPS for 2 days and expression of surface maturation markers was determined by flow cytometry. White curves are cells stained with FITC labeled isotype control, red curves are cells stained either CD40, CD86, or MHC class II specific mAb.

SHP-1 Inhibition in DCs Modulates their Activation, Migration and T Cell Stimulatory Functions.

SHP-1 inhibits JAK/STAT, Akt and NFκB signaling pathways among others in macrophages, T cells and B cells. Since these pathways are known to be critical in DC function and SHP-1 is highly expressed in DC, it indicates that SHP-1 is an important target for manipulating the efficacy of DC cell-based vaccines. To determine the effects of SHP-1 signaling in DC, bone marrow cells are isolated from C57BL/6 mice and cultured in GM-CSF and IL-4 to promote the differentiation of DC. CD11c⁺ DC, purified DC from this culture are transduced with an adenoviral vector encoding SHP-1 specific shRNAs, or a phosphatase dead dominant negative mutant of SHP-1(dnSHP-1) to inhibit SHP-1 and compared to non-transduced cells. SHP-1 effects on DC maturation and activation are determined at both basal levels and following the ligation of traditional activating receptors. DC maturation and activation are monitored by surface markers and Th1/2 cytokine expression. The rate of antigen processing is compared in transduced and non-transduced DC using ovalbumin protein (OVA) as a model antigen. DC loaded with whole OVA protein is monitored over time for the surface evolution of the MHC class 1 K^{b} immunodominant epitope, SIINFEKL using a K^{b}-SIINFEKL specific mAb. The ability of DC to phagocytose antigen is tested by DC uptake of fluorescent labeled beads. The effect of SHP-1 signaling on DC migration to draining lymph nodes is determined in vitro by two chamber migration assays and in vivo using CFSE labeled DC. The effect of SHP-1 signaling on the induction CD4⁺ and CD8⁺ T cell proliferation is determined using cocultures of CFSE labeled syngeneic CD3⁺ splenocytes and DC loaded with whole ovalbumin protein as a model antigen. The induction of CD4⁺ CD25⁺ regulatory T cells is determined in these cocultures by flow cytometry. The effector function of CD8⁺ CTL stimulated by SHP-1 transduced DC compared to non-transduced cells is determined by ⁵¹Cr release and antigen specific CTL precursor frequency monitored using K^{b}-SIINFEKL-tetramers.

### Inhibiting SHP-1 Signaling in DC Cell-Based Vaccines Modulates Anti-Tumor T Cell Responses Against TRAMP-Derived Prostate Cancer Cells in Ectopic Tumor Models and Against Spontaneously Developing Autochthonous Prostate Tumors in TRAMP Mice.

As an initial proof of concept that SHP-1 inhibition enhances DC signaling in vivo, bone marrow derived DC vaccines from SHP-1 deficient mice (C57BL/6J-Ptpn6me-v/J) are compared with the wild type derived C57BL/6J vaccine in their ability to promote tumor regression of subcutaneous ectopic (e.t.) tumors. Three e.t tumor models which have defined antigens, vary in their immunogenicity and growth rates, and which are progressively more difficult to treat are used. These models are: the highly immunogenic thymoma EG.7-OVA (expressing OVA); the fast growing and weakly immunogenic melanoma B16 (expressing tyrosinase-related protein 2 TRP-2 antigens); and very aggressive prostate adenocarcinoma TRAMP-C2 (expressing SPAS-1 antigen). Tumor lines that stably express a red-shifted luciferase that allows for monitoring the size and location of model tumors in living animals using IVIS™ optical bioluminescence imaging were created. Vaccines generated from bone marrow derived DC are prepared as described above and loaded with the appropriate antigens. Vaccine efficacy is determined by monitoring tumor growth and/or spread over time and by the expansion of antigen-specific CD8⁺ CTL quantified by ELISPOT assay or tetramer staining and CTL lytic function is measured by ⁵¹Cr release assays. Once the effect of SHP-1 inhibition alone in DC vaccines is determined, the efficacy of SHP-1 inhibition in combination with a known DC activating modification, a chimeric inducible CD40 construct (iCD40) is tested. The most effective of these SHP-1 inhibition vectors and/or the combination of SHP-1 inhibition and iCD40 against e.t tumors, is tested in TRAMP mice that develop spontaneous orthotropic prostate tumors at 3-6 months of age. The ability for genetically enhanced DC vaccines to prevent prostate tumors when TRAMP mice are vaccinated before 3 months of age or the ability to regress existing prostate tumors in protocols where mice are vaccinated after 4 months of age is determined.

### The Role of SHP-1 in DC Maturation.

To determine if SHP-1 influences DC maturation, purified differentiated DC that have not undergone any exposure to cytokine or TLR ligand maturation are transduced with the SHP-1 shRNA or the dnSHP-1 adenoviral constructs and returned to culture in complete media. At intervals of 8, 24, 48, or 72 hours, cells are analyzed for maturation markers by flow cytometry (I-A^{b}, CD40, CD80, CD86, CCR7). As a control for adenoviral specific influences on DC maturation these experiments include untransduced DC and DC transduced with a control adenoviral construct expressing an irrelevant gene, bacterial β-galactosidase (β-gal).

The Role of SHP-1 in DC Matured by TLR and/or Cytokines.

The experiments in described above are repeated but following viral transduction with SHP-1 inhibiting constructs the DC are pretreated with the TLR ligand LPS. Cells are sampled at timed intervals, as above, and analyzed for expression of maturation surface markers. In addition, supernatants from cell cultures at each sampling interval are collected to analyze for the expression of Th1 cytokines, IL-12p35, IL-12p40, IL-12p70, IL-6, IFNγ and the anti-Th1 cytokine, IL-10. IL-12p35 and IL-12p40 are the monomers that make up the active Th1 driving heterodimer, IL-12p70. IL-12p35 is expression is enhanced by CD40 ligation on DC where IL-12p40 is induced predominantly by TLR engagement (Schulz et al., 2000). Thus, the effect of SHP-1 inhibition on the signaling through pathways initiated by other classic DC ligands, CD40L, TNFα, IL-1β and IL-10 is examined. These experiments characterize the role of SHP-1 in modulating DC maturation driven by classical stimuli.

Since the efficacy of DC to mount a potent Th1 immune response may be dependent on their longevity in the lymph node where they encounter T cells, it is necessary to determine the effect of SHP-1 on the lifespan of DC. Typically, DC have a lifespan of 3-5 days after migrating to the lymph nodes (Hermans et al., 2000). By comparing the viability of SHP-1 inhibitor transduced and matured DC with those transduced with cells not expressing SHP-1 inhibition, over time this important functional question is addressed. SHP-1 transduced DC are exposed to a maturation cocktail of (LPS, CD40L, TNFα, and IL-1β) and cultured in complete media. Cells are monitored daily by flow cytometry (PI and annexin-5 staining) to determine the proportion of apoptotic cells.

For a DC vaccine to be effective, DC injected into a patient must migrate from the sight of injection to the draining lymph nodes in order to initiate a T cell response. DC migration to lymph nodes is controlled by the chemotactic receptor CCR7 which is up-regulated on activated DC (Riol-Blanco et al., 2005). CCR7 is a G protein coupled receptor that has downstream signaling through PI3K, MAPK, and Rho/Rac pathways in response to its cognate ligands chemokine CCL19 and CCL21 that are expressed in the lymph node. Since SHP-1 can potentially inhibit PI3K and members of the Rho pathway (Vav and Pyk2), will examine the effects SHP-1 inhibition in DC stimulated with CCR7 ligands. Both in vitro and in vivo migration assays are performed to determine the effects of SHP-1 inhibition.

### The Role of SHP-1 in DC Antigen Presentation.

Antigen presentation is one of the key functions of dendritic cells and is essential to their ability to initiate a T cell response (Banchereau et al., 2000). Antigen up-take an processing are known to be affected by cytokine signaling and thus may be influenced by SHP-1 activity in DC (Nguyen et al., 2002). To determine if SHP-1 inhibits the ability of DC to acquire and process antigen, purified DC are matured, transduced with one of the SHP-1 inhibiting constructs and loaded with whole ovalbumin protein (OVA). The dominant MHC class I peptide derived from OVA is the amino acid sequence SIINFEKL (positions 257-264 in the OVA protein) which binds to K^{b} (Shastri and Gonzalez, 1993). The K^{b}-SIINFEKL epitope can be specifically detected at the cell surface using the mAb 25.D1.16. (Germain et al., 1997). OVA loaded and transduced DC are assayed at intervals of 1, 4, 8, 24, 48, or 72 hours for the presence and magnitude of the K^{b}-SIINFEKL epitope. If SHP-1 affects the ability of DC to process antigen then differences in the surface expression of the K^{b}-SIINFEKL epitope will be apparent when SHP-1 transduced DC are compared with DC transduced with adenoviral β-gal. These experiments are carried out for several days in order to determine if SHP-1 effects are transient or long lasting.

The effect of SHP-1 inhibition on the ability of DC to take up antigen by phagocytosis is determined by incubating purified activated DC with fluorescent labeled latex bead. Each bead has a sufficient signal to be detected by flow cytometry. Phagocytosis can be quantified by integer beads signals (1 bead=x fluorescence, 2 beads=2x fluorescence) which show up as discrete peaks on a one dimensional histogram.

The Role of SHP-1 Expressed y DC in their Ability to Activate T Cells.

The ultimate test of DC function is their ability to stimulate antigen specific T cell proliferation and activation. To test the effect of SHP-1 in this process, transduced DC are loaded with OVA protein and co-cultured with purified syngeneic CD3⁺ T cells. Prior to co-culture, T cells are labeled with the fluorescent lipophilic dye CFSE (Carter et al., 2002). Upon each cell division the CFSE fluorescent signal is diluted by half and thus the number of divisions a cell has undergone since being stained can be determined by flow cytometry. By quantifying T cell proliferation in this manner, the proportion of the population dividing in response to DC stimulation with OVA antigens is determined. The phenotype of the proliferating T cells is determined by staining the various CFSE-low populations (proliferating cells) for the T cell subset markers CD4 and CD8. The frequency of SIINFEKL specific CD8⁺ T cells in the population responding to DC stimulation is also determined by flow cytometry staining of T cells with a fluorescently labeled tetrameric recombinant construct of K^{b}-SIINFEKL (tetramers). Since the ability of DC to initiate a prolonged CD8⁺ T cell response can be inhibited by the generation of CD4⁺CD25⁺ T regulatory cells (T reg), and SHP-1 can influence T reg differentiation (Carter et al., 2005), the CFSE proliferating population for CD25 expression is characterized. By comparing DC transduced with SHP-1 or β-gal the effects of SHP-1 on T cell activation are defined.

### Production of Bone Marrow Derived DC

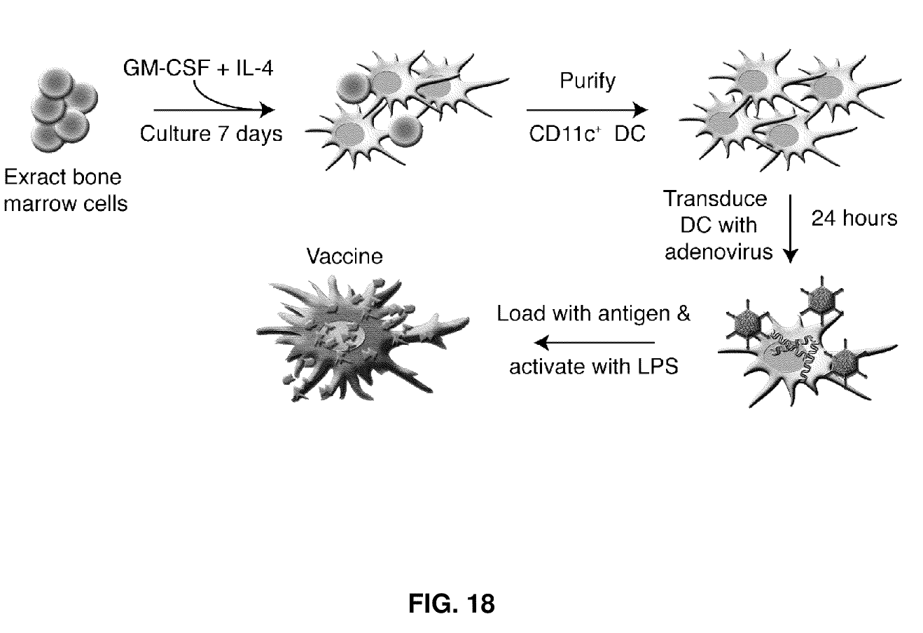

C57BL/6 bone marrow cells are extracted by flushing femurs and tibias with balanced salt solution. Bone marrow lymphocytes are isolated on a Lympholyte™ (Cedarlane) gradient, washed and cultured in complete DC media containing GM-CSF and IL-4 at 37° C. in humidified 5% CO₂. Every two days, the suspension cells are removed and replaced with fresh media for 7 days. Cultured cells are fractionated by passing through a MACS (magnetic sorting) anti-CD11c beads (Miltenyi Biotech). This process yields ≧94% CD11c⁺ DC (Hanks et al., 2005). Purified DC are analyzed for surface marker expression (I-A^{b}, CD40, CD80, CD86) by flow cytometry to give a baseline with which to compare changes due to future manipulations of the cells. See FIG. 18.

### Maturation and Antigen Loading of DC

Purified DC are incubated for 24 hours in the presence of a cocktail of mediators of maturation (LPS, CpG DNA, TNFα and CD40L) and in the presence of SIINFEKL peptide or whole ovalbumin as model antigens. DC maturation are quantified by flow cytometry for surface markers: I-A^{b}, K^{b}, CD40, CD80, CD86, CCR7. The ability of DC to process antigen is determined using the mAb 25.D1.16 that recognizes an epitope composed of SIINFELK peptide bound to the class I molecule K^{b} (Porgador et al., 1997).

### Adenoviral Transduction of DC

Initial testing is carried out with each adenoviral construct to determine the optimum MOI for transduction of SHP-1 shRNA or the dnSHP-1 construct. Optimum MOI of shRNA is determined by western blot for decreases in SHP-1 expression compared to transduction with an adenoviral construct expression 1-gal. Optimum expression of dnSHP-1 is determined by western blot detecting an HA-tag contained in the construct. Purified DC are incubated with adenovirus for 4 hours in complete media and the washed to remove unincorporated virus prior to functional experiments.

### Flow Cytometry

Typically, 10,000-20,000 events are measured from a cohort of at least 10⁶ stained cells. All cells are counter-stained with propidium iodide to identify viable DCs, and FITC or phycoerythrin (PE)-labeled antibodies to maturation markers or antigen processing markers are used with standard staining protocols. Non-specific binding is measured using PE (or FITC)-labeled isotype matched controls.

### Migration Assays

DC transduced with SHP-1 inhibiting vectors or irrelevant vector are incubated for 24 hours in complete media containing LPS, CD40L, TNFα, and IL-1β. For in vitro assays: cells are washed, labeled with CFSE and placed in the upper chamber of a 2 chamber 96 well plate separated by a FluoroBlok 8 μm pore membrane (BD Biosciences, CA) and the lower chamber contains complete media supplemented with CCL19. The FluoroBlok membrane does not permit fluorescent light transmission through the membrane so fluorescence of CFSE labeled cells that migrate through to the bottom chamber can be detected by a bottom-reading plate reader. Cell migration is determined following a 4 hour incubation at 37° C. For in vivo assays: cells are washed, labeled with CFSE and injected s.c. into the hind footpad of mice. At daily intervals mice are sacrificed and the popliteal lymph nodes that drain the hind extremities are removed, disaggregated and analyzed by flow cytometry for the presence of CFSE labeled cells.

### T Cell Assays

Splenocytes from C57BL/6 mice are separated by adherence to plastic culture dishes for 24 hours in complete media. The non-adherant fraction represents predominantly lymphocytes (T and B cells). Non-adherant splenocytes are incubated with activated, SHP-1 transduced, antigen loaded DC for 7 days. After 7 days expanded/viable T cells are Ficoll-purified and re-stimulated with activated, SHP-1 transduced, antigen loaded DC for 7 days. Following this second stimulation T cells are analyzed for proliferation by CSFE labeling and incubation with an antigen-pulsed cell line RMA-S. Proliferation is determined by the integral reduction in CFSE label per cell after a period of 4 days. The lytic activity of CTL is determined following the second stimulation T cells which are incubated with ⁵¹Cr labeled antigen-pulsed RMA-S cells and ⁵¹Cr release measured by standard methods. The induction of CD4⁺ CD25⁺ regulatory T cells is determined in DC stimulated splenocytes at each stimulation an aliquot of cells is analyzed by flow cytometry for the presence of CD3⁺CD4⁺CD25⁺ cells. In proliferation assays the proportion of proliferating CD25+ cells is determined by staining CFSE labeled cells with a PE-conjugated anti-CD25 antibody.

### Determine SHP-1 Inhibition in DC Vaccine Efficacy Against Ectopic Tumors.

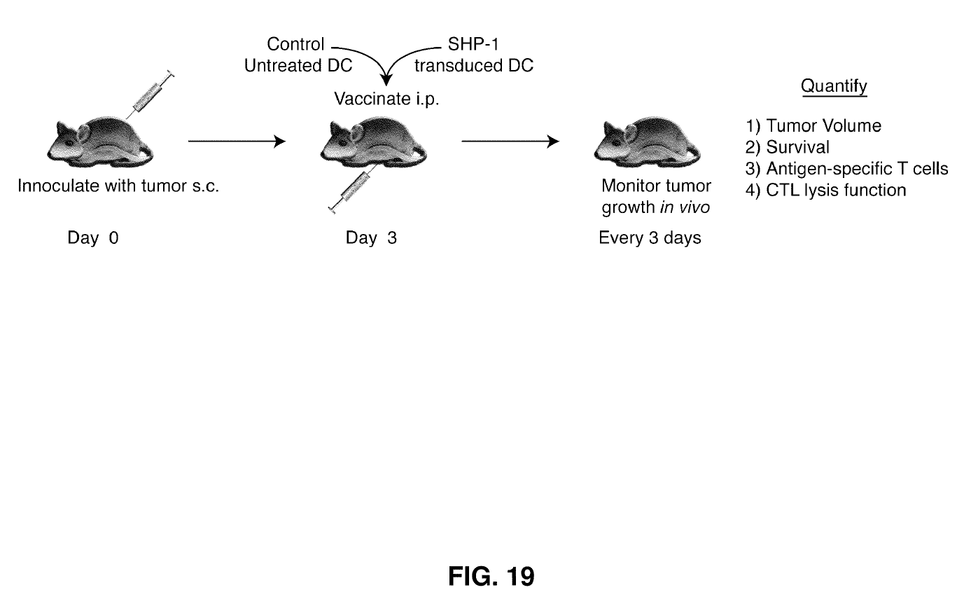

To determine if the SHP-1 inhibition in DC is able to enhance the efficacy of an anti-tumor vaccine, three well characterized ectopic tumor models, EG.7-OVA, B16 and TRAMP-C2 in C57BL/6 mice are used. The EG.7-OVA tumors express the OVA dominant peptide SIINFEKL and are highly immunogenic. The B16 model is a melanoma which is both aggressive and poorly immunogenic. B16 cells express tyrosinase related protein-2 (TRP-2) of which the TRP-2 peptide is recognized by CD8⁺ T cells in the context of K^{b}. B16 represents a much more difficult tumor to treat than the EG.7-OVA tumors. TRAMP tumors are very aggressive and non-immunogenic. By employing these three models, a range of anti-tumor efficacy generated by SHP-1 inhibition in DC is delineated. Variants of EG.7-OVA and B16 tumor lines that express a red-shifted click beetle luciferase (rs-Luc) to enable in vivo quantification of subcutaneous tumors using a non-invasive IVIS™ imaging system have been created. The TRAMP-C2 line is transduced with the same luciferase vector. DC is matured, transduced and activated and loaded with either SIINFEKL peptide (EG.7-OVA tumors) or TRP-2 peptide (B16 tumors) or SPAS-1 peptide (TRAMP-C2). To generate the ectopic tumors, wild type C57BL/6 mice are injected s.c. on the right side of the back with 10⁵ luciferase expressing tumor cells 3 days before vaccination. For vaccination 2×10⁶ purified, DC are injected i.p. into mice bearing the appropriate subcutaneous tumors. Mice are monitored for 3-4 weeks or until tumors reach 2 cm³ (FIGS. 18 and 19). At the end of each experiment T cell function assays are performed by 51Cr release and tumor antigen specific precursor frequency by ELISPOT or tetramer staining with the appropriate peptide tetramer combination.

Determine the Efficacy of SHP-1 Inhibition in DC Vaccine in Conjunction with a Stimulating Inducible CD40 (iCD40) Construct Against Ectopic Tumors.

It is possible that SHP-1 inhibition alone does not enhance anti-tumor responses above the level of control transduced DC. There is a novel DC activation system based on the CD40 signaling pathway to extend the pro-stimulatory state of DCs within lymphoid tissues (US Patent Pub No. US 2004/0209836, incorporated by reference in its entirety). A recombinant receptor has been engineered that is comprised of the cytoplasmic domain of CD40 fused to ligand binding domains and a membrane-targeting sequence (iCD40). The activation of CD40-dependent signaling cascades is regulated with a lipid-permeable, dimerizing drug (Amara et al., 1997). It was also demonstrated that peptide-pulsed iCD40-transduced bone marrow-derived DC can eliminate EG.7-OVA tumors in the presence of systemically injected dimerizer drug (Spencer et al., 2005). Transduction of DC with iCD40 has also been shown to extend the lifespan of the DC by approximately 2 fold. In addition to iCD40, a constitutively active chimeric variant of the signaling molecule Akt (myr_{F}-ΔAkt) that has shown promise in the regression of EG.7-OVA tumors has also been developed. SHP-1 has been shown to inhibit the native signaling pathways of both CD40 (O'Sullivan and Thomas, 2003) and Akt (Mills et al., 1999) and indicates that a DC vaccine is enhanced by combining these modifications. The combined effects of iCD40 and SHP-1 inhibition or myr_{F}-ΔAkt and SHP-1 inhibition with the individual modifications either iCD40, myr_{F}-ΔAkt or SHP-1 inhibition alone are compared using the ectopic tumor models.

### Determine the Efficacy of DC Vaccines Against Spontaneously Developing Orthotopic Prostate Tumors in TRAMP Mice.

TRAMP mice develop spontaneous prostate tumors in 3-6 months (Greenberg et al., 1999). Two strategies are tested: prevention of tumor development and regression of existing tumors. To test prevention efficacy, 8-week-old TRAMP mice are vaccinated before autochthonous tumors are detectable with DC (the best vaccine enhancement modification determined against e.t. tumors) loaded with SPAS-1 peptide antigen. Animals are euthanized at 6 months and prostate tumors evaluated. Further, T cell proliferation and CTL assays are performed to assess the adaptive immune response to SPAS-1. To determine vaccine efficacy against existing autochthonous prostate tumors, 4 month-old TRAMP mice are vaccinated as above. Survival and tumor size are monitored for 3 months. T cell function is measured when each animal is euthanized or at the conclusion of the experimental period.

### Ectopic Tumor Models.

C57BL/6 mice are injected s.c. with 10⁵ either EG.7-OVA, B16, or TRAMP-C2 tumor cells expressing rs-Luc. After 3 days most mice will develop a non-palpable tumor detectable by IVIS imaging. Administration of the DC vaccine occurs on Day 3 after the tumor inoculation. To image the tumors, mice are injected i.p. with d-luciferin, anesthetized with isofluorane placed in the imager for 1-5 minutes. Pixel volume can be quantified as a densitometric measure of tumor size. Mice are imaged every two days and euthanized if tumors reach 2 cm³ in volume. Survival is quantified as the days post-tumor inoculation until tumor reaches 2 cm³ and the mouse is euthanized.

### Statistical Tests and Power Calculations.

In order to achieve an 80% power of demonstrating significance (p<0.05) when the difference between these two groups is ≧25%, 5-8 mice are required per group in the e.t. tumor experiments where non-transduced vaccine controls virtually all develop 2 cm³ tumors by the end of 4 weeks. In the autochthonous prostate tumor model a larger number of animals are used (25 per group) since tumor size and the rate of tumor induction are significantly more varied than in the e.t. models.

Again, this invention is based on the emerging technology of DC based vaccines in the treatment of cancer, in certain embodiments. First generation DC vaccines, which were simply DC loaded with tumor antigens, showed limited efficacy against tumors but did show that this type of therapy could up-regulate tumor antigen specific T cell responses. Currently, 2^{nd} generation DC vaccines, that utilize cytokine enhancement to deliver antigen and increase its processing and presentation are in phase III clinical trials. These 2^{nd} generation vaccines show some limited tumor regression and have increased survival of patients by several months. In specific embodiments, 3^{rd} generation cancer vaccines are employed where DC are genetically enhanced to inhibit signaling pathways that in nature serve to regulate DC function and dampen immune responses. This dampening of immune activation maintains tolerance and prevents autoimmunity, but also permits tumors from escaping immune surveillance. The Src homology region 2 domain-containing phosphatase-1 (SHP-1) has been chosen as a target that is highly expressed in DC and that potentially antagonizes a number of activation pathways critical for DC function. This strategy is used in an effort to increase the activation, antigen presentation, migration and T cell activation abilities of DC and ultimately improve the efficacy of treatment for prostate cancer.

The approach of this invention is to enhance DC function by genetic modification to inhibit the signaling mechanisms that, in nature, dampen immune responses and prevent autoimmunity. A target is the hematopoietic phosphatase, SHP-1, which is known to antagonize a multitude of stimulatory pathways crucial for DC function. This strategy increases the activation, antigen presentation, migration and T cell activation abilities of DC and ultimately improve the efficacy of treatment for cancer, for example.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

### PATENTS AND PATENT APPLICATIONS

UK Patent No. GB 2,397,818 B
U.S. Pat. No. 4,683,195
U.S. Pat. No. 4,683,202
U.S. Pat. No. 5,910,488
U.S. Pat. No. 5,962,428
U.S. Pat. No. 5,998,203
U.S. Pat. No. 6,573,099
U.S. Pat. No. 6,753,423
U.S. Patent Publication No. 20030130186
U.S. Patent Publication No. 20040110296
U.S. Patent Publication No. 20040167090
U.S. Patent Publication No. 20040192626
U.S. Patent Publication No. 20040249178
U.S. Patent Publication No. 20050043219
U.S. Patent Publication No. 20050074771
U.S. Patent Publication No. 20050107325
U.S. Patent Publication No. 20050119470
U.S. Patent Publication No. 20050153337
U.S. Patent Publication No. 20050158727
U.S. Patent Publication No. 20050239739
U.S. Patent Publication No. 20050282188

### PUBLICATIONS

Allan, D. S., A. J. McMichael, and V. M. Braud. 2000. The ILT family of leukocyte receptors. Immunobiology 202:34.
Amara, J. F., T. Clackson, V. M. Rivera, T. Guo, T. Keenan, S, Natesan, R. Pollock, W. Yang, N. L. Courage, D. A. Holt, and M. Gilman. 1997. A versatile synthetic dimerizer for the regulation of protein-protein interactions. Proc Natl Acad Sci USA 94:10618.
Ausubel et al., Current Protocols in Molecular Biology (1994).
Banchereau, J., and A. K. Palucka. 2005. Dendritic cells as therapeutic vaccines against cancer. Nat Rev Immunol 5:296.
Banchereau, J., and R. M. Steinman. 1998. Dendritic cells and the control of immunity. Nature 392:245.
Banchereau, J., F. Briere, C. Caux, J. Davoust, S. Lebecque, Y. J. Liu, B. Pulendran, and K. Palucka. 2000. Immunobiology of dendritic cells. Annu Rev Immunol 18:767.
Bander, N. H., D. Yao, H. Liu, Y. T. Chen, M. Steiner, W. Zuccaro, and P. Moy. 1997///Dec. 1. MHC class I and II expression in prostate carcinoma and modulation by interferon-alpha and -gamma. Prostate 33:233.
Beaucage et al., Tetrahedron 49:1925 (1993).
Brummelkamp et al., Science 296:550 (2002).
Cambier, J. C., 1997. Inhibitory receptors abound? Proc Natl Acad Sci USA 94:5993.
Carter, J. D., G. M. Calabrese, M. Naganuma, and U. Lorenz. 2005. Deficiency of the Src homology region 2 domain-containing phosphatase 1 (SHP-1) causes enrichment of CD4⁺CD25⁺ regulatory T cells. J Immunol 174:6627.
Carter, L., L. A. Fouser, J. Jussif, L. Fitz, B. Deng, C. R. Wood, M. Collins, T. Honjo, G. J. Freeman, and B. M. Carreno. 2002. PD-1:PD-L inhibitory pathway affects both CD4⁺ and CD8⁺ T cells and is overcome by IL-2. Eur J Immunol 32:634.
Cella, M., A. Engering, V. Pinet, J. Pieters, and A. Lanzavecchia. 1997. Inflammatory stimuli induce accumulation of MHC class II complexes on dendritic cells. Nature 388:782.
Cella, M., M. Salio, Y. Sakakibara, H. Langen, I. Julkunen, and A. Lanzavecchia. 1999. Maturation, activation and protection of dendritic cells induced by double-stranded RNA. J Exp Med 189:821.
Cuevas, B., Y. Lu, S. Watt, R. Kumar, J. Zhang, K. A. Siminovitch, and G. B. Mills. 1999. SHP-1 regulates Lck-induced phosphatidylinositol 3-kinase phosphorylation and activity. J Biol Chem 274:27583.
Donze et al., Nucleic Acids Res. 30:e46 (2002).
Evel-Kabler, K., and S. Y. Chen. 2006. Dendritic Cell-Based Tumor Vaccines and Antigen Presentation Attenuators. Mol. Ther.
Gingrich, J. R., R. J. Barrios, B. Foster, and N. M. Greenberg. 1999. Pathologic progression of autochthonous prostate cancer in the TRAMP model. Prostate Cancer and Prostatic Diseases 2:70.
Gubler and Hoffman, Gene, 25:263-269 (1983).
Gupta, N., A. M. Scharenberg, D. N. Burshtyn, N. Wagtmann, M. N. Lioubin, L. R. Rohrschneider, J. P. Kinet, and E. O. Long. 1997. Negative signaling pathways of the killer cell inhibitory receptor and Fc gamma RIIb1 require distinct phosphatases. J Exp Med 186:473.
Hanks, B. A., J. Jiang, R. A. Singh, W. Song, M. Barry, M. H. Huls, K. M. Slawin, and D. M. Spencer. 2005. Re-engineered CD40 receptor enables potent pharmacological activation of dendritic-cell cancer vaccines in vivo. Nat Med 11:130.
Hermans, I. F., D. S. Ritchie, J. Yang, J. M. Roberts, and F. Ronchese. 2000. CD8⁺ T cell-dependent elimination of dendritic cells in vivo limits the induction of antitumor immunity. J Immunol 164:3095.
Hou, W. S., and L. Van Parijs. 2004. A Bcl-2-dependent molecular timer regulates the lifespan and immunogenicity of dendritic cells. Nat Immunol 5:583.
Hunziker et al., Nucleic Acid Analogues: Synthesis and Properties in Modern Synthetic Methods VCH 331-417 (1995).
Innis et al, PCR Protocols: A Guide to Methods and Applications (1990).
, Janeway, C. A., Jr., 2001. How the immune system protects the host from infection. Microbes Infect 3:1167.
Kantoff, P., 2005. Recent progress in management of advanced prostate cancer. Oncology (Williston Park) 19:631.
Kriegler, Gene, Transfer and Expression: A Laboratory Manual (1990).
Langenkamp, A., M. Messi, A. Lanzavecchia, and F. Sallusto. 2000. Kinetics of dendritic cell activation: impact on priming of TH1, TH2 and nonpolarized T cells. Nat Immunol 1:311.
Lee et al., Nat. Biotech. 20:500 (2002).
Lock, K., J. Zhang, J. Lu, S. H. Lee, and P. R. Crocker. 2004. Expression of CD33-related signals on human mononuclear phagocytes, monocyte-derived dendritic cells and plasmacytoid dendritic cells. Immunobiology 209:199.
Long, E. O., 1999. Regulation of immune responses through inhibitory receptors. Annu Rev Immunol 17:875.
Macvicar, G. R., and M. Hussain. 2005. Chemotherapy for prostate cancer: implementing early systemic therapy to improve outcomes. Cancer Chemother Pharmacol:1.
Matthews, R. J., D. B. Bowne, E. Flores, and M. L. Thomas. 1992. Characterization of hematopoietic intracellular protein tyrosine phosphatases: description of a phosphatase containing an SH2 domain and another enriched in proline-glutamic acid- serine- and threonine-rich sequences. Mol Cell Biol 12:2396.
Mesmaeker et al., Novel Backbone Replacements for Oligonucleotides in Carbohydrate Modifications in Antisense Research ACS 24-39 (1994).
Minoo, P., M. M. Zadeh, R. Rottapel, J. J. Lebrun, and S. Ali. 2004. A novel SHP-1/Grb2-dependent mechanism of negative regulation of cytokine-receptor signaling: contribution of SHP-1 C-terminal tyrosines in cytokine signaling. Blood 103:1398.
Miyagishi et al., Nat. Biotech. 20:497 (2002).
Napolitani, G., A. Rinaldi, F. Bertoni, F. Sallusto, and A. Lanzavecchia. 2005. Selected Toll-like receptor agonist combinations synergistically trigger a T helper type 1-polarizing program in dendritic cells. Nat Immunol 6:769.
Nestle, F. O., J. Banchereau, and D. Hart. 2001. Dendritic cells: On the move from bench to bedside. Nat Med 7:761.
Nguyen, L. T., S. Radhakrishnan, B. Ciric, K. Tamada, T. Shin, D. M. Pardoll, L. Chen, M. Rodriguez, and L. R. Pease. 2002. Cross-linking the B7 family molecule B7-DC directly activates immune functions of dendritic cells. J Exp Med 196:1393.
O'Sullivan, B., and R. Thomas. 2003. CD40 and dendritic cell function. Crit. Rev Immunol 23:83.
Overwijk, W. W., D. S. Lee, D. R. Surman, K. R. Irvine, C. E. Touloukian, C. C. Chan, M. W. Carroll, B. Moss, S. A. Rosenberg, and N. P. Restifo. 1999. Vaccination with a recombinant vaccinia virus encoding a “self” antigen induces autoimmune vitiligo and tumor cell destruction in mice: requirement for CD4⁺ T lymphocytes. Proc Natl Acad Sci USA 96:2982.
Paddison et al., Genes Dev. 16:948 (2002).
Paul et al., Nat. Biotech. 20:505 (2002).
Petrylak, D. P., C. M. Tangen, M. H. Hussain, P. N. Lara, Jr., J. A. Jones, M. E. Taplin, P. A. Burch, D. Berry, C. Moinpour, M. Kohli, M. C. Benson, E. J. Small, D. Raghavan, and E. D. Crawford. 2004. Docetaxel and estramustine compared with mitoxantrone and prednisone for advanced refractory prostate cancer. N Engl J Med 351:1513.
Pirtskhalaishvili, G., G. V. Shurin, A. Gambotto, C. Esche, M. Wahl, Z. R. Yurkovetsky, P. D. Robbins, and M. R. Shurin. 2000///Aug. 15. Transduction of dendritic cells with Bcl-xL increases their resistance to prostate cancer-induced apoptosis and antitumor effect in mice. J Immunol 165:1956.
Porgador, A., J. W. Yewdell, Y. Deng, J. R. Bennink, and R. N. Germain. 1997. Localization, quantitation and in situ detection of specific peptide-MHC class I complexes using a monoclonal antibody. Immunity 6:715.
Qasimi, P., and A. L. Mui. 2006. Divergent mechanisms utilized by SOCS3 to mediate interleukin-10 inhibition of tumor necrosis factor alpha and nitric oxide production by macrophages. J Biol Chem 281:6316.
Ravetch, J. V., 1997. Fc receptors. Curr Opin Immunol 9:121.
Ridgway, D., 2003. The first 1000 dendritic cell vaccines. Cancer Invest 21:873.
Riol-Blanco, L., N. Sanchez-Sanchez, A. Torres, A. Tejedor, S, Narumiya, A. L. Corbi, P. Sanchez-Mateos, and J. L. Rodriguez-Fernandez. 2005. The chemokine receptor CCR7 activates in dendritic cells two signaling modules that independently regulate chemotaxis and migratory speed. J Immunol 174:4070.
Sallusto, F., B. Palermo, D. Lenig, M. Miettinen, S. Matikainen, I. Julkunen, R. Forster, R. Burgstahler, M. Lipp, and A. Lanzavecchia. 1999. Distinct patterns and kinetics of chemokine production regulate dendritic cell function. Eur J Immunol 29:1617.
Sambrook et al., Molecular Cloning A Laboratory Manual (2nd ed. 1989).
Scaringe et al., Nuc. Acids Res. 18:5433 (1990).
Schuler, G., and R. M. Steinman. 1997 Oct. 20. Dendritic cells as adjuvants for immune-mediated resistance to tumors. J Exp Med 186:1183.
Schuler-Thurner, B., E. S. Schultz, T. G. Berger, G. Weinlich, S. Ebner, P. Woerl, A. Bender, B. Feuerstein, P. O. Fritsch, N. Romani, and G. Schuler. 2002. Rapid induction of tumor-specific type 1 T helper cells in metastatic melanoma patients by vaccination with mature cryopreserved peptide-loaded monocyte-derived dendritic cells. J Exp Med 195:1279.
Schulz, O., A. D. Edwards, M. Schito, J. Aliberti, S. Manikasingham, A. Sher, and C. Reis e Sousa. 2000. CD40 triggering of heterodimeric IL-12p70 production by dendritic cells in vivo requires a microbial priming signal. Immunity 13:453.
Shastri, N., and F. Gonzalez. 1993. Endogenous generation and presentation of the ovalbumin peptide/Kb complex to T cells. J Immunol 150:2724.
Small, E. J., P. Fratesi, D. M. Reese, G. Strang, R. Laus, M. V. Peshwa, and F. H. Valone. 2000. Immunotherapy of hormone-refractory prostate cancer with antigen-loaded dendritic cells. J Clin Oncol 18:3894.
Stebbins, C. C., C. Watzl, D. D. Billadeau, P. J. Leibson, D. N. Burshtyn, and E. O. Long. 2003. Vav1 dephosphorylation by the tyrosine phosphatase SHP-1 as a mechanism for inhibition of cellular cytotoxicity. Mol Cell Biol 23:6291.
Strengell, M., A. Lehtonen, S. Matikainen, and I. Julkunen. 2006. IL-21 enhances SOCS gene expression and inhibits LPS-induced cytokine production in human monocyte-derived dendritic cells. J Leukoc Biol.

Su, Z., J. Dannull, B. K. Yang, P. Dahm, D. Coleman, D. Yancey, S. Sichi, D. Niedzwiecki, D. Boczkowski, E. Gilboa, and J. Vieweg. 2005. Telomerase mRNA-transfected dendritic cells stimulate antigen-specific CD8⁺ and CD4⁺ T cell responses in patients with metastatic prostate cancer. J Immunol 174:3798.
Sui et al., Proc. Natl. Acad. Sci. USA 99:5515 (2002).
Suzue, K., X. Zhou, H. N. Eisen, and R. A. Young. 1997. Heat shock fusion proteins as vehicles for antigen delivery into the major histocompatibility complex class I presentation pathway. Proc Natl Acad Sci USA 94:13146.
Thomas, M. L., 1995. Of ITAMs and ITIMs: turning on and off the B cell antigen receptor. J Exp Med 181:1953.
Thurner, B., C. Roder, D. Dieckmann, M. Heuer, M. Kruse, A. Glaser, P. Keikavoussi, E. Kampgen, A. Bender, and G. Schuler. 1999. Generation of large numbers of fully mature and stable dendritic cells from leukapheresis products for clinical application. J Immunol Methods 223:1.
Tsui, H. W., K. A. Siminovitch, L. de Souza, and F. W. Tsui. 1993. Motheaten and viable motheaten mice have mutations in the haematopoietic cell phosphatase gene. Nat Genet. 4:124.
Usman et al., J. Am. Chem. Soc. 109:7845 (1987).
Vieweg, J., and A. Jackson. 2005. Modulation of antitumor responses by dendritic cells. Springer Semin Immunopathol 26:329.
Viviani, V. R., A. Uchida, W. Viviani, and Y. Ohmiya. 2002. The influence of Ala243 (Gly247), Arg215 and Thr226 (Asn230) on the bioluminescence spectra and pH-sensitivity of railroad worm, click beetle and firefly luciferases. Photochem Photobiol 76:538.
Wincott et al., Methods Mol. Bio. 74:59 (1997).
Wincott et al, Nuc. Acids Res. 23:2677-2684 (1995).
Yeung, Y. G., Y. Wang, D. B. Einstein, P. S. Lee, and E. R. Stanley. 1998. Colony-stimulating factor-1 stimulates the formation of multimeric cytosolic complexes of signaling proteins and cytoskeletal components in macrophages. J Biol Chem 273:17128.
Yokoyama, W. M., 1998. Natural killer cell receptors. Curr Opin Immunol 10:298.
Yu et al., Proc. Natl. Acad. Sci. USA 99:6047 (2002).
Zhang, J., A. K. Somani, and K. A. Siminovitch. 2000. Roles of the SHP-1 tyrosine phosphatase in the negative regulation of cell signaling. Semin Immunol 12:361.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of enhancing a dendritic cell based vaccine for an individual, comprising administering to the individual a SHP-1 modulatory agent.

2. The method of claim 1, wherein the SHP-1 modulatory agent is delivered into a dendritic cell from the individual ex vivo, and the cell is administered to the individual.

3. The method of claim 1, further comprising the step of delivering the SHP-1 modulatory agent to a dendritic cell of the individual.

4. The method of claim 1, wherein the SHP-1 modulatory agent is uptaken by a dendritic cell of the individual in vivo.

5. The method of claim 4, wherein the SHP-1 modulatory agent is delivered to the individual in a vector.

6. The method of claim 5, wherein the vector is an adenoviral vector.

7. The method of claim 5, wherein the vector further comprises a promoter active in dendritic cells.

8. The method of claim 1, wherein the SHP-1 modulatory agent is a SHP-1 inhibitory agent.

9. The method of claim 1, wherein the SHP-1 modulatory agent is a SHP-1 stimulatory agent.

10. The method of claim 1, wherein the cell further comprises an antigen.

11. The method of claim 10, wherein said antigen is a tumor antigen.

12. The method of claim 1, wherein the SHP-1 modulatory agent is comprised in a vector.

13. The method of claim 1, wherein the individual has cancer.

14. The method of claim 1, wherein the individual has a disease caused by a pathogen.

15. The method of claim 9, wherein the individual has an autoimmune disease.

16. The method of claim 1, wherein the dendritic cell based vaccine is administered to the individual simultaneously or subsequently to the administration of a cancer treatment.

17. A method of producing a dendritic cell based vaccine, comprising delivering a SHP-1 modulatory agent to a dendritic cell.

18. The method of claim 17, wherein the SHP-1 modulatory agent is a SHP-1 stimulatory agent.

19. The method of claim 17, wherein the SHP-1 modulatory agent is a SHP-1 inhibitory agent.

20. The method of claim 17, wherein the dendritic cell further comprises an antigen.

21. A dendritic cell based vaccine composition comprising a SHP-1 modulatory agent.

22. The composition of claim 21, further comprising an antigen.

23. A method of modulating an immune response in an individual comprising delivering to the individual a SHP-1 modulatory agent.

24. The method of claim 23, wherein the SHP-1 modulatory agent is delivered to the individual in a dendritic cell from the individual.


SEQUENCE LISTING (21 sequences)

SEQ ID NO: 1 - protein, 8 aa, Artificial Sequence
  synthetic peptide
SIINFEKL

SEQ ID NO: 2 - protein, 8 aa, Artificial Sequence
  synthetic peptide
RSYRYKLL

SEQ ID NO: 3 - protein, 8 aa, Artificial Sequence
  synthetic peptide
LTFLYTLL

SEQ ID NO: 4 - protein, 9 aa, Artificial Sequence
  synthetic peptide
VSKINRTEM

SEQ ID NO: 5 - protein, 9 aa, Artificial Sequence
  synthetic peptide
LLLGTVHAL

SEQ ID NO: 6 - protein, 9 aa, Artificial Sequence
  synthetic peptide
AQMNNRDCL

SEQ ID NO: 7 - DNA, 1815 bp, Artificial Sequence
  Synthetic construct
atgtacccatatgacgttccagactacgcggtgaggtggtttcaccgggacctcagcgggcctgatgcagagaccctgctaaagggccggggagtccctgggagcttcctggctcggcccagccgcaagaaccagggtgacttctccctctcagtcagggtggatgatcaggtgactcatattcggatccagaactcaggggacttctatgacctgtacggaggggagaagtttgcgacgctgacagagctggtcgagtattacacgcagcagcagggcatcctgcaggaccgagatggcaccatcatccaccttaagtacccactgaactgctcggaccccaccagtgagaggtggtaccacggccacatatctggagggcaggcggagtcactgctgcaggccaagggcgagccctggacatttcttgtgcgtgagagtctcagccaacctggtgattttgtgctctctgtgctcaatgaccagcccaaggctggcccaggttccccgctcagggtcactcatatcaaggttatgtgtgagggtggacgctatactgtgggtggctcagagacgtttgacagcctcacagacctggtggagcacttcaagaagacagggattgaggaggcctcgggtgcctttgtctacctgcggcagccttactacgccactcgggtaaacgcagctgacattgagaatcgggtcttggaactgaacaagaagcaggagtcggaggacacagccaaggctggcttctgggaggagtttgagagtctacaaaagcaggaggtaaagaatctacaccaacgtctggaagggcagcggccggagaacaagagcaagaaccgctacaagaacattcttccctttgaccacagccgagtgatcctgcagggacgtgacagtaacatcccaggctctgactacatcaatgccaactacgtgaagaaccagctgctaggtccagatgagaactctaagacctacatcgccagccagggctgtctggatgccacagtcaatgacttctggcagatggcttggcaggagaacactcgtgtcatcgtcatgactaccagagaggtggagaaaggccggaacaaatgtgtcccatactggcccgaggtgggcactcagcgtgtctatggtctctactctgtgaccaacagtagggagcatgacacagcagaatacaaactgcgaacattacagatctccccactagacaatggggacctggttcgggagatatggcactaccagtacctgagctggcctgaccatggggttcccagtgagcctgggggtgtcctcagctttctggatcagatcaaccagcgacaggaaagtttgcctcatgcagggcccatcattgtgcattgcagcgctggcatcggccgcacgggcaccatcatcgtcattgatatgcttatggaaagcatctccaccaaggggctagactgtgacattgatatccagaagaccatccagatggtacgagcacagcgctccggcatggtgcagaccgaggcccagtacaagtttatttacgtggccattgcccagttcatcgaaacgaccaagaagaaactggagatcatacaatcccagaagggccaggagtcggagtatgggaatatcacgtaccctcccgctgtgaggagtgcccacgccaaagcctcgcgtacttcctccaagcacaaggaggaggtgtacgaaaacgtgcatagcaagagcaagaaggaagagaaagtaaagaagcagcggtcggcagacaaggataagaacaaaggttctctcaagaggaagtga

SEQ ID NO: 8 - DNA, 1815 bp, Artificial Sequence
  Synthetic construct
atgtacccatatgacgttccagactacgcggtgaggtggtttcaccgggacctcagcgggcctgatgcagagaccctgctgaagggccggggagtccctgggagcttcctggctcggcccagccgcaagaaccagggtgacttctccctctcagtcagggtggatgatcaggtgactcatattcggatccagaactcaggggacttctatgacctgtacggaggggagaagtttgcgacgctgacagagctggtcgagtattacacgcagcagcagggcatcctgcaggaccgagatggcaccatcatccaccttaagtacccactgaactgctcggaccccaccagtgagaggtggtaccacggccacatatctggagggcaggcggagtcactgctgcaggccaagggcgagccctggacatttcttgtgcgtgagagtctcagccaacctggtgattttgtgctctctgtgctcaatgaccagcccaaggctggcccaggttccccgctcagggtcactcatatcaaggttatgtgtgagggtggacgctatactgtgggtggctcagagacgtttgacagcctcacagacctggtggagcacttcaagaagacagggattgaggaggcctcgggtgcctttgtctacctgcggcagccttactacgctactcgggtaaacgcagctgacattgagaatcgggtcttggaactgaacaagaagcaggagtcggaggacacagccaaggctggcttctgggaggagtttgagagtctacaaaagcaggaggtaaagaatctacaccaacgtctggaagggcagcggccagagaacaagagcaagaaccgctacaagaacattcttccctttgaccacagccgagtgatcctgcagggacgtgacagtaacatcccaggctctgactacatcaatgccaactacgtgaagaaccagctgctaggtccagatgagaactctaagacctacatcgccagccagggctgtctggatgccacagtcaatgacttctggcagatggcttggcaggagaacactcgtgtcatcgtcatgactaccagagaggtggagaaaggccggaacaaatgtgtcccatactggcccgaggtgggcactcagcgtgtctatggtctctactctgtgaccaacagtagggagcatgacacagcagaatacaaactgcgaacattacagatctccccactagacaatggggacctggttcgggagatatggcactaccagtacctgagctggcctgaccatggggttcccagtgagcctgggggtgtcctcagctttctggatcagatcaaccagcgacaggaaagtttgcctcatgcagggcccatcattgtgcattccagcgctggcatcggccgcacgggcaccatcatcgtcattgatatgcttatggaaagcatctccaccaaggggctagactgtgacattgatatccagaagaccatccagatggtacgagcacagcgctccggcatggtgcagaccgaggcccagtacaagtttatttacgtggccattgcccagttcatcgaaacgaccaagaagaaactggagatcatacaatcccagaagggccaggagtcggagtatgggaatatcacgtaccctcccgctgtgaggagtgcccacgccaaagcctcgcgtacttcctccaagcacaaggaggaggtgtacgaaaacgtgcatagcaagagcaagaaggaagagaaagtaaagaagcagcggtcggcagacaaggagaagaacaaaggttctctcaagaggaagtga

SEQ ID NO: 9 - DNA, 1503 bp, Artificial Sequence
  Synthetic construct
atgtacccatatgacgttccagactacgcgttgtcccgcgggtggtaccacggccacatatctggagggcaggcggagtcactgctgcaggccaagggcgagccctggacatttcttgtgcgtgagagtctcagccaacctggtgattttgtgctctctgtgctcaatgaccagcccaaggctggcccaggttccccgctcagggtcactcatatcaaggttatgtgtgagggtggacgctatactgtgggtggctcagagacgtttgacagcctcacagacctggtggagcacttcaagaagacagggattgaggaggcctcgggtgcctttgtctacctgcggcagccttactacgctactcgggtaaacgcagctgacattgagaatcgggtcttggaactgaacaagaagcaggagtcggaggacacagccaaggctggcttctgggaggagtttgagagtctacaaaagcaggaggtaaagaatctacaccaacgtctggaagggcagcggccagagaacaagagcaagaaccgctacaagaacattcttccctttgaccacagccgagtgatcctgcagggacgtgacagtaacatcccaggctctgactacatcaatgccaactacgtgaagaaccagctgctaggtccagatgagaactctaagacctacatcgccagccagggctgtctggatgccacagtcaatgacttctggcagatggcttggcaggagaacactcgtgtcatcgtcatgactaccagagaggtggagaaaggccggaacaaatgtgtcccatactggcccgaggtgggcactcagcgtgtctatggtctctactctgtgaccaacagtagggagcatgacacagcagaatacaaactgcgaacattacagatctccccactagacaatggggacctggttcgggagatatggcactaccagtacctgagctggcctgaccatggggttcccagtgagcctgggggtgtcctcagctttctggatcagatcaaccagcgacaggaaagtttgcctcatgcagggcccatcattgtgcattgcagcgctggcatcggccgcacgggcaccatcatcgtcattgatatgcttatggaaagcatctccaccaaggggctagactgtgacattgatatccagaagaccatccagatggtacgagcacagcgctccggcatggtgcagaccgaggcccagtacaagtttatttacgtggccattgcccagttcatcgaaacgaccaagaagaaactggagatcatacaatcccagaagggccaggagtcggagtatgggaatatcacgtaccctcccgctgtgaggagtgcccacgccaaagcctcgcgtacttcctccaagcacaaggaggaggtgtacgaaaacgtgcatagcaagagcaagaaggaagagaaagtaaagaagcagcggtcggcagacaaggagaagaacaaaggttctctcaagaggaagtga

SEQ ID NO: 10 - DNA, 2168 bp, Mus musculus
ccacgcgtccgcaggactgcaggttggctttggaggcctgggctctgagagccttgcctgaggctcatctctagagtttgtacgtgcctgcccagacaaactgttccctccacattttctgcagccaattcagtgagaaccccaggatggtgaggtggtttcaccgggacctcagcgggcctgatgcagagaccctgctaaagggccggggagtccctgggagcttcctggctcggcccagccgcaagaaccagggtgacttctccctctcagtcagggtggatgatcaggtgactcatattcggatccagaactcaggggacttctatgacctgtacggaggggagaagtttgcgacgctgacagagctggtcgagtattacacgcagcagcagggcatcctgcaggaccgagatggcaccatcatccaccttaagtacccactgaactgctcggaccccaccagtgagaggtggtaccacggccacatatctggagggcaggcggagtcactgctgcaggccaagggcgagccctggacatttcttgtgcgtgagagtctcagccaacctggtgattttgtgctctctgtgctcaatgaccagcccaaggctggcccaggttccccgctcagggtcactcatatcaaggttatgtgtgagggtggacgctatactgtgggtggctcagagacgtttgacagcctcacagacctggtggagcacttcaagaagacagggattgaggaggcctcgggtgcctttgtctacctgcggcagccttactacgccactcgggtaaacgcagctgacattgagaatcgggtcttggaactgaacaagaagcaggagtcggaggacacagccaaggctggcttctgggaggagtttgagagtctacaaaagcaggaggtaaagaatctacaccaacgtctggaagggcagcggccggagaacaagagcaagaaccgctacaagaacattcttccctttgaccacagccgagtgatcctgcagggacgtgacagtaacatcccaggctctgactacatcaatgccaactacgtgaagaaccagctgctaggtccagatgagaactctaagacctacatcgccagccagggctgtctggatgccacagtcaatgacttctggcagatggcttggcaggagaacactcgtgtcatcgtcatgactaccagagaggtggagaaaggccggaacaaatgtgtcccatactggcccgaggtgggcactcagcgtgtctatggtctctactctgtgaccaacagtagggagcatgacacagcagaatacaaactgcgaacattacagatctccccactagacaatggggacctggttcgggagatatggcactaccagtacctgagctggcctgaccatggggttcccagtgagcctgggggtgtcctcagctttctggatcagatcaaccagcgacaggaaagtttgcctcatgcagggcccatcattgtgcattgcagcgctggcatcggccgcacgggcaccatcatcgtcattgatatgcttatggaaagcatctccaccaaggggctagactgtgacattgatatccagaagaccatccagatggtacgagcacagcgctccggcatggtgcagaccgaggcccagtacaagtttatttacgtggccattgcccagttcatcgaaacgaccaagaagaaactggagatcatacaatcccagaagggccaggagtcggagtatgggaatatcacgtaccctcccgctgtgaggagtgcccacgccaaagcctcgcgtacttcctccaagcacaaggaggaggtgtacgaaaacgtgcatagcaagagcaagaaggaagagaaagtaaagaagcagcggtcggcagacaaggataagaacaaaggttctctcaagaggaagtgatctgggcattcgtctgcaggtggccatgcatcagccttgatccctgcagaggcttccaccggatagactgagacctgaggccctcaccagaccctaggaccacccccatcttcttgtaatttaagtgactgtggtcatctgaatctgtatatagcccggcaagtccccagggagagccgggcccttctattcttgtaaataaattccctggaccactgaaaaaaaaaaaaaaaa

SEQ ID NO: 11 - DNA, 50 bp, Artificial Sequence
  shRNA
caccggagcatgacacagcagaatacgaatattctgctgtgtcatgctcc

SEQ ID NO: 12 - DNA, 50 bp, Artificial Sequence
  shRNA
caccgcaccatcatccaccttaagtcgaaacttaaggtggatgatggtgc

SEQ ID NO: 13 - protein, 9 aa, Artificial Sequence
  synthetic peptide
SVYDFFVWL

SEQ ID NO: 14 - DNA, 2211 bp, Mus musculus
ccttcctgtctccgccctgctggctgccccaggccagtagagtggtagcccggggagcaggactgcaggttggctttggaggcctgggctctgagagccttgcctgaggctcatctctagagtttgtacgtgcctgcccagacaaactgttccctccacattttctgcagccaattcagtgagaaccccaggatggtgaggtggtttcaccgggacctcagcgggcctgatgcagagaccctgctgaagggccggggagtccctgggagcttcctggctcggcccagccgcaagaaccagggtgacttctccctctcagtcagggtggatgatcaggtgactcatattcggatccagaactcaggggacttctatgacctgtacggaggggagaagtttgcgacgctgacagagctggtcgagtattacacgcagcagcagggcatcctgcaggaccgagatggcaccatcatccaccttaagtacccactgaactgctcggaccccaccagtgagaggtggtaccacggccacatatctggagggcaggcggagtcactgctgcaggccaagggcgagccctggacatttcttgtgcgtgagagtctcagccaacctggtgattttgtgctctctgtgctcaatgaccagcccaaggctggcccaggttccccgctcagggtcactcatatcaaggttatgtgtgagggtggacgctatactgtgggtggctcagagacgtttgacagcctcacagacctggtggagcacttcaagaagacagggattgaggaggcctcgggtgcctttgtctacctgcggcagccttactacgctactcgggtaaacgcagctgacattgagaatcgggtcttggaactgaacaagaagcaggagtcggaggacacagccaaggctggcttctgggaggagtttgagagtctacaaaagcaggaggtaaagaatctacaccaacgtctggaagggcagcggccagagaacaagagcaagaaccgctacaagaacattcttccctttgaccacagccgagtgatcctgcagggacgtgacagtaacatcccaggctctgactacatcaatgccaactacgtgaagaaccagctgctaggtccagatgagaactctaagacctacatcgccagccagggctgtctggatgccacagtcaatgacttctggcagatggcttggcaggagaacactcgtgtcatcgtcatgactaccagagaggtggagaaaggccggaacaaatgtgtcccatactggcccgaggtgggcactcagcgtgtctatggtctctactctgtgaccaacagtagggagcatgacacagcagaatacaaactgcgaacattacagatctccccactagacaatggggacctggttcgggagatatggcactaccagtacctgagctggcctgaccatggggttcccagtgagcctgggggtgtcctcagctttctggatcagatcaaccagcgacaggaaagtttgcctcatgcagggcccatcattgtgcattgcagcgctggcatcggccgcacgggcaccatcatcgtcattgatatgcttatggaaagcatctccaccaaggggctagactgtgacattgatatccagaagaccatccagatggtacgagcacagcgctccggcatggtgcagaccgaggcccagtacaagtttatttacgtggccattgcccagttcatcgaaacgaccaagaagaaactggagatcatacaatcccagaagggccaggagtcggagtatgggaatatcacgtaccctcccgctgtgaggagtgcccacgccaaagcctcgcgtacttcctccaagcacaaggaggaggtgtacgaaaacgtgcatagcaagagcaagaaggaagagaaagtaaagaagcagcggtcggcagacaaggagaagaacaaaggttctctcaagaggaagtgatctgggcattcgtctgcaggtggccatgcatcagccttgatccctgcagaggcttccacccgatagactgagacctgtggccctcaccagaccctaggaccacccccatcttcttgtaatttaagtgactgtggtcatctgaatctgtatatagcccggcaagtccccagggagagccgggcccttctattcttgtaaataaattccctggaccactgtaaaaaaaaaaaa

SEQ ID NO: 15 - DNA, 2222 bp, Mus musculus
gttcttctctcagagacctgtaatccaggggcttggcctcagagtcccattggtttgacaggctggatagaggaggaagtggccaagaaacgaaatatattcttcctgaaggtctggatccctgaacagctgtgccactcgattggccccgcccccgtcgccctttgcctgtgacttctccccactcctcccgggagatgttgtcccgcgggtggtttcaccgggacctcagcgggcctgatgcagagaccctgctgaagggccggggagtccctgggagcttcctggctcggcccagccgcaagaaccagggtgacttctccctctcagtcagggtggatgatcaggtgactcatattcggatccagaactcaggggacttctatgacctgtacggaggggagaagtttgcgacgctgacagagctggtcgagtattacacgcagcagcagggcatcctgcaggaccgagatggcaccatcatccaccttaagtacccactgaactgctcggaccccaccagtgagaggtggtaccacggccacatatctggagggcaggcggagtcactgctgcaggccaagggcgagccctggacatttcttgtgcgtgagagtctcagccaacctggtgattttgtgctctctgtgctcaatgaccagcccaaggctggcccaggttccccgctcagggtcactcatatcaaggttatgtgtgagggtggacgctatactgtgggtggctcagagacgtttgacagcctcacagacctggtggagcacttcaagaagacagggattgaggaggcctcgggtgcctttgtctacctgcggcagccttactacgctactcgggtaaacgcagctgacattgagaatcgggtcttggaactgaacaagaagcaggagtcggaggacacagccaaggctggcttctgggaggagtttgagagtctacaaaagcaggaggtaaagaatctacaccaacgtctggaagggcagcggccagagaacaagagcaagaaccgctacaagaacattcttccctttgaccacagccgagtgatcctgcagggacgtgacagtaacatcccaggctctgactacatcaatgccaactacgtgaagaaccagctgctaggtccagatgagaactctaagacctacatcgccagccagggctgtctggatgccacagtcaatgacttctggcagatggcttggcaggagaacactcgtgtcatcgtcatgactaccagagaggtggagaaaggccggaacaaatgtgtcccatactggcccgaggtgggcactcagcgtgtctatggtctctactctgtgaccaacagtagggagcatgacacagcagaatacaaactgcgaacattacagatctccccactagacaatggggacctggttcgggagatatggcactaccagtacctgagctggcctgaccatggggttcccagtgagcctgggggtgtcctcagctttctggatcagatcaaccagcgacaggaaagtttgcctcatgcagggcccatcattgtgcattgcagcgctggcatcggccgcacgggcaccatcatcgtcattgatatgcttatggaaagcatctccaccaaggggctagactgtgacattgatatccagaagaccatccagatggtacgagcacagcgctccggcatggtgcagaccgaggcccagtacaagtttatttacgtggccattgcccagttcatcgaaacgaccaagaagaaactggagatcatacaatcccagaagggccaggagtcggagtatgggaatatcacgtaccctcccgctgtgaggagtgcccacgccaaagcctcgcgtacttcctccaagcacaaggaggaggtgtacgaaaacgtgcatagcaagagcaagaaggaagagaaagtaaagaagcagcggtcggcagacaaggagaagaacaaaggttctctcaagaggaagtgatctgggcattcgtctgcaggtggccatgcatcagccttgatccctgcagaggcttccacccgatagactgagacctgtggccctcaccagaccctaggaccacccccatcttcttgtaatttaagtgactgtggtcatctgaatctgtatatagcccggcaagtccccagggagagccgggcccttctattcttgtaaataaattccctggaccactgtaaaaaaaaaaaa

SEQ ID NO: 16 - DNA, 2321 bp, Homo sapien
gctctaaaacgagaagtacaagtgagttcccccaaggggtcggccgcgcctcttcctgtccccgccctgccggctgccccaggccagtggagtggcagccccagaactgggaccaccgggggtggtgaggcggcccggcactgggagctgcatctgaggcttagtccctgagctctctgcctgcccagactagctgcacctcctcattccctgcgcccccttcctctccggaagcccccaggatggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtgagcggtgctgtcctcaggtggccatgcctcagccctgaccctgtggaagcatttcgcgatggacagactcacaacctgaacctaggagtgccccattcttttgtaatttaaatggctgcatcccccccacctctccctgaccctgtatatagcccagccaggccccaggcagggccaacccttctcctcttgtaaataaagccctgggatcactgtgaaaaaaaaaaaaaaaaaaaaaaaaaaaaaaaaaaaaaaaaaaaaaaaaaaaaaaaaaaaaaaaaaaaaaaaaaaa

SEQ ID NO: 17 - DNA, 2464 bp, Homo sapien
gctctaaaacgagaagtacaagtgagttcccccaaggggtcggccgcgcctcttcctgtccccgccctgccggctgccccaggccagtggagtggcagccccagaactgggaccaccgggggtggtgaggcggcccggcactgggagctgcatctgaggcttagtccctgagctctctgcctgcccagactagctgcacctcctcattccctgcgcccccttcctctccggaagcccccaggatggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaagagcttggagtctagtgcagggaccgtggctgcgtcacctgtgagacggggtggccagaggggactgccagtgccgggtccccctgtgctgtctcctgacctgcaccaactgcctgtacttgcccccctgcacccggctgcagacacaaggaggatgtgtatgagaacctgcacactaagaacaagagggaggagaaagtgaagaagcagcggtcagcagacaaggagaagagcaagggttccctcaagaggaagtgagcggtgctgtcctcaggtggccatgcctcagccctgaccctgtggaagcatttcgcgatggacagactcacaacctgaacctaggagtgccccattcttttgtaatttaaatggctgcatcccccccacctctccctgaccctgtatatagcccagccaggccccaggcagggccaacccttctcctcttgtaaataaagccctgggatcactgtgaaaaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 2234 bp, Homo sapien
attggcctggcaggcaggatcgaggaggaagtggctgattactgagcggttcttcctcacctggcttgggccactgtgcacagctgtgccgctggctcagccccgccccctgcggccctctgccgtggcttccccctccctacagagagatgctgtcccgtgggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtgagcggtgctgtcctcaggtggccatgcctcagccctgaccctgtggaagcatttcgcgatggacagactcacaacctgaacctaggagtgccccattcttttgtaatttaaatggctgcatcccccccacctctccctgaccctgtatatagcccagccaggccccaggcagggccaacccttctcctcttgtaaataaagccctgggatcactgtgaaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 2220 bp, Homo sapien
caggatcgaggaggaagtggctgattactgagcggttcttcctcacctggcttgggccactgtgcacagctgtgccgctggctcagccccgccccctgcggccctccgccgtggcttccccctccctacagagagatgctgtcccgtgggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtgagcggtgctgtcctcaggtggccatgcctcagccctgaccctgtggaagcatttcgcgatggacagactcacaacctgaacctaggagtgccccattcttttgtaatttaaatggctgcatcccccccacctctccctgaccctgtatatagcccagccaggccccaggcagggccaacccttctcctcttgtaaataaagccctgggatcactgtgaaaaaaaaaaaaaaaaaaaaaaaaaaaaaaaaaaaaaaaaaaaaaaaaaaaaaaaaaaaaaaaaaaaaaaaaaaa

SEQ ID NO: 20 - DNA, 5802 bp, Artificial Sequence
  Synthetic construct
ggtaccttttcctacagaactgtaacacttccactagggaagccttcccctcagagctgtaacatctacaggcaataaatatgcagagagtgaggaacttgggagtagtcagtctcaatagggcgttttcaccaaactgtcccctcagggatccatgcagaagaggaggtggacagactgtaggccagaggtgatgtaaggaggagctggagaacaggctgaaaggctgccaaatgccattctctgcgtgggataaaccgttgcaattgctaactcacagcaatggcagtcgccttcatggggtccacgcaagactggccctatctgcaatcagtcacagaaggggaagggactcatgggactctaccactgacctctgagctctgccataggtttgggctgctttcaatctggtcgcagaagcttgtttttgtttttccatgggtggtggttagtgagaaactcttactggtggtcatggtgctaagaataaatgctgttgtgtacttagtcttaaagaggtcacttagtcattcgcccactccaaccataaagctcaagaaacattttcagaagggggaacaaaaagtgtaggtactggagggtggggaagagtgtgcaaaatgctgtcttctgaacatgacatagctctctctcttatgaagtcatagctacggccacttgtgtaggatcaagcatggatgggaaggggcttgtgaaggcccactcccagctgagaagctatagtatggtagtatggctgatggctggtgagggaggagggaggtcacttttctttagtgggggtgtggctgttggtaggttgcccatgctctagtgagtagcctacagccatgtgaatgcaggctgtactaattaaacttagtggattaaaaaaaaagggactatgaaggtaggagagagatatattgaaggatgttttgaaggagcaagaggggagagtttgggaagaatatgatcaagatacattgtgaatatatatatatatatatatatatatatatataaaatttgttgaagaattaaaaaattgtaagaacacctgaaattttatttcttaaataatcaagggtaataaagaaatgctttgtgttgatctaatatatctaatatatgtaaaaccctttaattagatgaattactatggatttcttttccaggaacattcttttttgttgttttaaaagtaatttactgcattattaattaattaattaattaatgcacatgcctgtgtgtgtatacatatgtgcccatgtatactcacacacgcaggaaaaataaggaaaactctttaataatcatgtgtgactgcttctggctaatgcacaaaagagagtactgaaccaaacccacatcagctcgtacagtttcacagctccatgttgcatttgagcaactaaacatgtttcacagaggcagacattccaaggtttgcttgtttggagaaataggaggacttacgatgtaaaatatgggcagagctatcctggttttcccaggcaactgttagccccgggccatcttgtctgcctggcaggatatgtgctccttcctgacaagagcttgtggagtgctccttcctgacaagagcgcggggagtgctccttcctgacaagagcgcgggggagtgcttcttaagccaaattcttcctcgtgtgacttaatatttcctgagcacgtttagcagatcttttacaacaacaatgtaatattctcatcttatatcatggccacacgtgtgtgtgtgtgtgtgtgtgtgtgtgtgtgtgtgtgtgtgtgtgtgtgactacagtgccccacaacaaagacaagtgtctccctggaacttgctaaaatggtgagttgcagttcagagagagaccctgtctcaaaacacaggatagaccccttttattttattcaagtaggggaagtgagactgtcacgcccgctctcgaccagcaagaacgacgcgaccaccagtccttctaacagcagtttattcagtcttcatctttcttctttctcttcatcagtaccgttccccagctgaagagttctgatccttctcaacagtctgttttacaggaacacttctttaccaccattccccgtgatgcagttctgaatcctccctgtagcagtgggtcttcgctcgtgactgaagatgtttcttttcccgggttttcagcaccacttgttgcgcgcgctcaacaggccaggaagaacgacgctgctacaggatccttctgcacacatttattcagtcctgtttcttctttctccatatatctcccttgtttttatatctcccttgtttatatatctcccttgtttttatatctcccttgtttatatatctcccccgaaccctgggcctctcactccttttatactctctctcatccacgcactgcaggccatgccccctcgccagtcacgaggcttcagctaatcagggcagcaggggcaaatctccaccaaattggattcacctgtatcctggtacacctgcgcagcactcaagatgtttgtgtcttatatgaggaagtcaggtgcaagtcatatgacttagctgcagtctctggcgcctttgggactgccgccacacccgctcctaacaaatacaggaaggggaggtcaaaaccaggagggttcgttagggaaggccatcagtgcagcaaacattcatgacatctctcttacgaagcttcccgtggctagctcctgaaagccatcaatcttagcccccaaactgtctgaaagcactgctccactgcacacagacatgagcaaacaagtctgtccatccaccctgggcctcaactggttctcaagatttcaagttcctctctaagatgtctgggtcccatctcttggcttaggtctgttggcataacagccccgtctggttccccactgtcctgggaaggaactctctgttcatctgcgtgaaatagaattttcttgtcttgctctcgaaccaggcagcctcagctgtgaacaacgcagaaatggtcaagattaaggctcgggacctgcacgacaagaagaaggaggagctgttgaaacaactggacgacctgaaggtggaattgtcccagcttcccgtcgccaaagtgacaggcagtgcagcgtccaagctctccaaccccaagtcctcactgttattaaccagactcaaaaagaaaacctcaggaaattctacaagggcaagaagtacaagcccctgggcctgtgacccaagaagactagagccatgcgtcacccgctcaccaaacacaaggagaagctgaagaccaagaagaagcagcggaaggagcggctgtacccactgcacaagtgtgcagtcaaggcctgagacgacaatgacaataaagtgcaagactgactggcaaaaaaaaaaaaagaattttcttgtctcttggacccggttccctctgcttctttcttaattagaaaagggggtaggttaggggtctggcggtgctaagtgtcatagtggtggtagaggggtgggctgggcagaagtgtcaggatgctggacaggcagtggcagtccttactttctgctccccgactttcagccagttataatgaccttgactgatggtcttcttcaagtcctggtacagacatagcgagtgttgtgccttgggctggttgcccatgtgtctgcctcccttccagggaaggacaaagaacagtatgcccagggacaaaaagcaaagactcagaaaacatgagaattgtttcttgaggccaaagctttcttttggaagggtgggatcccagagctggcctgctgtccagtggactctgcactggattcccctttcttgatgcctctcttttcttcccccaattctctcctcctctttcctattactacagctcattggccagtgggttttggctcccaaagacaaacaaagacagaaagatagcatactggccctttccagtccaatgctatgtggagatgctggcctttccccctggcagtctgctctctgctggtgcctcacacgggcatacactccgtaactcagatattaccaaccatgcacggtccttgcttttctttccttttcactttatggttcttttttttttcttttgtcttcattcttttctcatatactacatcctgacctcagtctccccccttcccagtccctccttaccacttcccctctctcctttcccttcagaaaagtgcaggctcccagggacatcaaccaaacctggttctctgaaaggtgaatgtgactatcttattcctcatgcatgacttctctacacttccttctgcctgcgggataaaaataaaagtctttcaaaactcccaggtcccgtggcctgctcactctccagttttccaacactccagcctcatctgttggcagtcttatctcccacgctgtcctgttaactctttatggtgtgccagagaaacaaggcctccagcaagaagtcactccacaaaatgaggaaaaataaggagaaaacctctaataaaaatagtttgtgtccagtttggctagtgcccaaagagaatccagtccagcatggaaattatttgaggcgtatgtgggtcttgagctacaaaacttcacctatgatatcacctgagtatctctctaaagtcccagccccaagttgcactctgaacattaaaacatgtttacagaaatgtatgcttagccattttagaccaaacgagatgccatcagtgttacctctaaagcaggtcaacataggaagatttgtactgtaaaggtactgtcacaaatgccagctacttagcaccccagttctttgctggctgctgctatccaaggatgttctggcggcctggaacaatcaaacttttcctgggagaggaatgagaaccagaactttcgatccaatgcttaccccaccccctctttggagtgcctgacagaggcaggttcttgataggagaaagggcctaatggccagctgtgggccagggttatcttccagaataaggtttaagggatgataatcctaatgaacatatccattgcttctgaaattcagtttttagtattctcttgaccttggctgcctttaagactcagctcaagtgctacttcccccagaaagcagtctgaggtcctctcggtctgagtaatacttctagtggttggtctctgtacctcttttctcacagggcccactttagtgttcacccatctgcttttactctgatctctgggttatgtttctcctatacctagcaaaaggctcagaccacccctcttgattatgttgagcaaatgactaatccactgaatgaatgaatgagtgagtgaatgaatgaatgaatgaatgaatgccagcctgtgctccctacatggatcatgtgcttacttcttagtctacttccaggccagaagtggagggctccgtcatctgttctctctcctcctgtggctgactcacacttcaaggtcaagggaaacttctgccagtacaaaagtctgagagggatcagataatccgggagtttacatatatccatccgggcaagaattggggaaccagaacaatatgtcaccaagtcgtttcaagtagagcaactcttccctggaagtgtgtaggctgcctcggtccccactctatccatttcatctcagtttgcccccacctcctctgagtcacgctgacaacttccctcctggtctctggcctcctgaccacctttcttctcatttgcttcttctgtggtgacttggcagctgtctccaagttgctcagagcctgcttctg

SEQ ID NO: 21 - DNA, 6000 bp, Homo sapien
acatgcactgccttgcacatgcagcacaaaggccctcaccagatgctggtgccatgttcttggacttcccagcctccagaaccacaaagcttctattgatacattacctagtttgcggtaatctgttatagcagcagggaacagacaaagacacactctatagttctgaacaatgaggacctcttatatgacctcaacaccattatcacacccaatgaaataagtgattccttggtattaagatcatagtcaaaattgtgtaactgtctcaaaaatatctttttacttctggtctatttgaacgaaaattcgaactaggtccatacatgatggttggttgttatttctgctgtctctcttagtctaatattcccagcggaagcgcattttgattaacgtataaaagaacacagcaggaaaggccatctcttgcatgtgaggggtgatcactgtcaatgaaagtgctcctagacatatcaaccaaatgtaatgggtggacctttgctgaatattgattttaacaaatgagctgtaaaataacatttttggagacaatctaagaaaatagaacatgaccagtatattggtatattaaggagtaattgttaattttgttggctgtgatttgttggttcttgtggttacatttaaaatacccttatctattggtggtatacactgaagtatttctagatgaaacaatatggtgtttggaatgtgctttaaagtgttcagaaagagaacatagcagaaacaagagtagaaaaatgttggttacttaaaatgagtgatggttatatggggttgattttccagtctctctattttttaatgtgttttaaattttccattataagaggcctttaaaataacaataaaaaggtgaacattttaatacatttgagatttctactttggaagaaagtttgaccagatagctatgggggagcttttaaaaacgtaaccttggctggacatggtggctcatacctgtaatcctagtgttttggggggacaagatgggaggattgcttgaggccaggagttcaagaccagcctgggcaatatagcgaggtcccatttataaaaaaatttttttaaaattagccaggcatggtggtggtgtgtgcctgtagtcctagctactcaggaggctgaggcaagaggatggcttgaggccagagtttgaggctgcagtgagctgtgattgcatcactgctttctagcctgggtgacagagtgagacctggtctcaaaaaacaaacaaacaaacaaaaacataagcttaaggtgggctccaggaagctttatcactacttcgtggcgtgtctttggaatgctgttatattaggttggtgcaaaagtaattgggtttttgccattgctttcaatttcaactaatactcctctactttttctcatgcctagaaacaagggcagctgcgttccctgcacctgacatgtgacagcgccccagttgggagccagggcacctggagcaccagctgcagaatcaaccacctcatcttccgtggcggcgcccaggtcagcctggcttctgtcccctcactgctcccctgccccaccctgtctttactgctctgtgacctctcagttccttttcctcagatcaccttcttggctacctttgacgtctcccccaaggctgtcctgggagaccggctgcttctgacagccaatgtgagcaggtgagccgggccaggccaggggcagtgcccctcatctccagcctcacaccccattctcctcctctggggcctctggcaactgagtctctcctctttctccagtgagaacaacactcccaggaccagcaagaccaccttccagctggagctcccggtgaagtatgctgtctacactgtggttagcaggtcagcaggtaccccactgcaggaaaaagggttcttctctctgacctcaaaaagaaaaaaaaaaaaaggccttgaaacgctgccacagagggtgagataaggtgtttgaaactaaaaggtcaggtgtttcagcagacaccttccttcagccaatgccttcctcgaatttgctgtgtgccaggcagggtgctgtggttattttccatacattcatttgacattcattgaagatttactgagcccccattatgtgtgatcaaaaccagacatgaaccctcgcccttgtggggtgtgccttgctggatgttctcctgtgcctggtgtttcccactctcacctgcacctgcatgcgtggaggctcccagccaggtgcacgctctgagctcgtgtgctggggtgtgccccaggctctcataccccttggcaggggacccacaggcagggctcacctactctaggcatgtgtggtccacagcttggccacacatggcaggtgtgatccacagcttggccacacatggcaggtgtgactctgtgggttagggcacagggagtgccaggttggggcatttctgggggaggccaaggtgggaggattgcttgaggccaggagttcaggatcagcctgggcaatatagtgagaccctagctctacaaaaaattggcagaggaggaatgaggcttcagagtaggcgtggtgggctggtgtttggactatgccagctggaggacaggtgccggaagagtgaagcagtgaggcagtggtgagactggccacctgtgtgatggtcccagctgcccaccaggtctccaggcttgtgcccaaaaagggctcaggaggtgttaaactgagacctcatgaagagtctgctgtgggccctccctcccctccaagccccaggcccggaaaagccaaggaaatgggaaaaaagggcccccggggaggtatgtgggggactgggaaggggcaatgctcagtagcatttggtcaacagcgtcagcctgggccaccccaactctgtgctgtgtcccacagagccctggcatcctacatccccacaaagggatggaaggaaagcggaggggcaggccatggcccctgctctcctgcagtcagtggcagagctctctgctccgttcattgtaccgactacccggccccactggcagcctcctctgcaggcagctatgagctctttgcctcatttttttttttttaaagcccaggtcttgctctgttgcccaggctggagtgcagtggtgtgatcacagctcacagcagccttgaactcctcttgggctcaagcgagcctcccacctcagcctcccaagtagttgagactacagatgtgtgccaccatgcctggctaatttaaaaaattgttttggagagatggggtcttgctatgttgcccaggctgatcttgaatcctggcctcaagtgatcctcccacctcaacctcccaaagtgctgggattgcaggtgtgagccactgcactcagccaattagcacttgtttgaagcccagccctgcttttctagactctctctctttttttttttttttgagaccaagtctcactgtgttgcccaggctggagtgcagttgtaccatctctgctcactgcaacctccgcctcctggctccaagcaattctcgtgcctcagcctcccaagtagctgggattacaggcacctgccaccacacctggctaatttttgtatttttagtagagatggtgtttctccatgttggccaggctggtcttgaactcccgacctcaggtgatctgcctgtctcagcctgtcaaagtgctgggattataggcgtgagcactgtgcctggtctctaaactctctctctctgtttttttttgagagagagagagtcttgctctgtcattcagtggcgtgatctgggctcactgcaacctctgtcccctgggctcaaacaattctcctgtctcagcctcctgagtagctgggattacaggtgcacaccatcatgcctggctaatttttgtatttttagtagagacagggtttcaccatgttggccagggtgatctcgaactcctgatctcaggtgatccgcccatctcagcatctcaaagtgctgggattacaggcctgagccaccgcgcctggcctctaaactctcttataacctaactcagccacagccctcattccaggacattccaaggccccaccgaccacctgtcctctcatgctctagccaatgccttctgcagatgccccatggtagttcacatccacttatgcgtcttctctctccagccacgaacaattcaccaaatacctcaacttctcagagtctgaggagaaggaaagccatgtggccatgcacagataccaggcaggtggtggagacgcaggagactgggctggggtgggaggctgggagccggagactggggagggatttgggctttggcgtgggctctgccctcagtgccctctgtgcaggtcaataacctgggacagagggacctgcctgtcagcatcaacttctgggtgcctgtggagctgaaccaggaggctgtgtggatggatgtggaggtctcccacccccaggtacccaaggactgcatgtggctcctccacgaatgccctttctacctggattccttgtgccccatgtgggtccctgatgtcccagctgagacacttgttctctgcattttcccccagaacccatcccttcggtgctcctcagagaaaatcgcacccccagcatctgacttcctggcgcacattcagaagaatcccgtgctggtgaggagggctctgggctggccctcactgtaggccccacatcagaggaatttaacccaggagttcatgttccatatccatcctgctgaagtaccctcttgcattcggatatggccgctgccctcaagtcacaccgcataatgctgcctcccaccttcacactcatctttctcagccccatgctatttatctgcccccaggactgctccattgctggctgcctgcggttccgctgtgacgtcccctccttcagcgtccaggaggagctggatttcaccctgaagggcaacctcagctttggctgggtccgccaggtgtgtgggtgcaacgacagagcccctgccccagactcaggcgggacctggcatgtctgtgcccatctgcaagccagggcacccccagagctctgagcctcccccagagccagttcaacaggtttcccccaacccctttgcagatattgcagaagaaggtgtcggtcgtgagtgtggctgaaattacgttcgacacatccgtgtactcccagcttccaggacaggaggcatttatgagagctcaggtagagaccatgtggagggcagcgaccaggctggaaagagggcccctagggctacatctgtggtgctgggtggggggtttgcaagccttgggggaggagggcgaaggcctctgggcaggatagctgtccctaagggcacgggtgctgctgtgtctcacctcttggagcagggcctggggaaggaggggagggagttaaaggttggggagcctgggaggagtctgggatagtaggaggatgggagtgctctgacagggtcacttccacttcagacgacaacggtgctggagaagtacaaggtccacaaccccacccccctcatcgtaggcagctccattgggggtctgttgctgctggcactcatcacagcggtactgtacaaagtgagtgttttatgccactcttgacaccaccagcatctggtcccgctctttttgcagagtgagaaggagctcactttgaaggcagaggcacattcttactgggtcacttcatatgagaaactgcttcccacctgcaatgtcacctgtccccagtggccccctgctttgtgattcccaggcttcctctaatatttctccctttctttcctgctcttctccatcattctacgtgtttcccttgacagcagattatcatataaaagcacagacctgggtttgaatgtcgacatcaccacgggttctttttgtctttgaccataggccagtgtctgctccactctgggccttgatttctcaatgtga